SEQUENCE LISTING (64 sequences)

SEQ ID NO: 1 - DNA, 16 bp, Artificial Sequence
  Description of Artificial Sequence Synthetic probe
agtggaaccgagatac

SEQ ID NO: 2 - DNA, 16 bp, Artificial Sequence
  Description of Artificial Sequence Synthetic probe
agtggaaccaagatac

SEQ ID NO: 3 - protein, 639 aa, Homo sapiens
MPQPSVSGMDPPFGDAFRSHTFSEQTLMSTDLLANSSDPDFMYELDREMNYQQNPRDNFLSLEDCKDIENLESFTDVLDNEGALTSNWEQWDTYCEDLTKYTKLTSCDIWGTKEVDYLGLDDFSSPYQDEEVISKTPTLAQLNSEDSQSVSDSLYYPDSLFSVKQNPLPSSFPGKKITSRAAAPVCSSKTLQAEVPLSDCVQKASKPTSSTQIMVKTNMYHNEKVNFHVECKDYVKKAKVKINPVQQSRPLLSQIHTDAAKENTCYCGAVAKRQEKKGMEPLQGHATPALPFKETQELLLSPLPQEGPGSLAAGESSSLSASTSVSDSSQKKEEHNYSLFVSDNLGEQPTKCSPEEDEEDEEDVDDEDHDEGFGSEHELSENEEEEEEEEDYEDDKDDDISDTFSEPGYENDSVEDLKEVTSISSRKRGKRRYFWEYSEQLTPSQQERMLRPSEWNRDTLPSNMYQKNGLHHGKYAVKKSRRTDVEDLTPNPKKLLQIGNELRKLNKVISDLTPVSELPLTARPRSRKEKNKLASRACRLKKKAQYEANKVKLWGLNTEYDNLLFVINSIKQEIVNRVQNPRDERGPNMGQKLEILIKDTLGLPVAGQTSEFVNQVLEKTAEGNPTGGLVGLRIPTSKV

SEQ ID NO: 4 - protein, 639 aa, Artificial Sequence
  Description of Artificial Sequence Synthetic polypeptide
MPQPSVSGMDPPFGDAFRSHTFSEQTLMSTDLLANSSDPDFMYELDREMNYQQNPRDNFLSLEDCKDIENLESFTDVLDNEGALTSNWEQWDTYCEDLTKYTKLTSCDIWGTKEVDYLGLDDFSSPYQDEEVISKTPTLAQLNSEDSQSVSDSLYYPDSLFSVKQNPLPSSFPGKKITSRAAAPVCSSKTLQAEVPLSDCVQKASKPTSSTQIMVKTNMYHNEKVNFHVECKDYVKKAKVKINPVQQSRPLLSQIHTDAAKENTCYCGAVAKRQEKKGMEPLQGHATPALPFKETQELLLSPLPQEGPGSLAAGESSSLSASTSVSDSSQKKEEHNYSLFVSDNLGEQPTKCSPEEDEEDEEDVDDEDHDEGFGSEHELSENEEEEEEEEDYEDDKDDDISDTFSEPGYENDSVEDLKEVTSISSRKRGKRRYFWEYSEQLTPSQQERMLRPSEWNQDTLPSNMYQKNGLHHGKYAVKKSRRTDVEDLTPNPKKLLQIGNELRKLNKVISDLTPVSELPLTARPRSRKEKNKLASRACRLKKKAQYEANKVKLWGLNTEYDNLLFVINSIKQEIVNRVQNPRDERGPNMGQKLEILIKDTLGLPVAGQTSEFVNQVLEKTAEGNPTGGLVGLRIPTSKV

SEQ ID NO: 5 - DNA, 7782 bp, Homo sapiens
gagtcacgcgatttccgggaacccgtcaggaaggacataaacaaaacaaacccgaggcagcatggagaggggccgtggcccctgcagcggaaccggacccagtccctgagccgcccctacacccacagacagcatcgcacagaattattttaaaaaaaagcagtgatccaagcaattgaattggaagcactctggggaaacctgctgtttattgtggaaatcatcttcgatcttggaattgaaagtaaagctggaaaggaatttacaaacaagaaaaaaaagaagtttggaatcggattcacaggatctgggcttggaaatgcctcagcctagtgtaagcggaatggatccgcctttcggggatgcctttcgaagccacaccttttcggaacaaactctgatgagcacagatctcttagcaaacagttcggatccagatttcatgtatgaactggatagagagatgaactaccaacagaatcctagagacaactttctttctttggaggactgcaaagacattgaaaatctggagtctttcacagatgtcctggataatgagggtgctttaacctcaaactgggaacagtgggatacatactgtgaagacctaacgaaatataccaaactaaccagctgtgacatctggggaacaaaagaagtggattacttgggtcttgatgacttttctagtccttaccaagatgaagaggttataagtaaaactccaactttagctcaacttaatagtgaggactcacagtctgtttctgattccctttattaccccgattcacttttcagtgtcaaacaaaatcccttaccctcttcattccctggtaaaaagatcacaagcagagcagctgctcctgtgtgttcttctaagactctgcaggctgaggtccctttgtcagactgtgtccaaaaagcaagtaaacccacttcaagcacacaaatcatggtgaagaccaacatgtatcataatgaaaaggtgaactttcatgttgaatgtaaagactatgtaaaaaaggcaaaggtaaagatcaacccagtgcaacagagccggcccttgttgagccagattcacacagatgcagcaaaggagaacacctgctactgtggtgcagtggcaaagagacaagagaaaaaagggatggagcctcttcaaggtcatgccactcccgctttgccttttaaagaaacccaggaactattactaagtcccctgccccaggaaggtcctgggtcacttgcagcaggagagagcagcagtctttctgccagtacatcagtctcagattcatcccagaaaaaagaagagcacaattattctctttttgtctccgacaacttgggtgaacagccaactaaatgcagtcctgaagaagatgaggaggacgaggaggatgttgatgatgaggaccatgatgaaggattcggcagtgagcatgaactgtctgaaaatgaggaggaggaagaagaggaagaggattatgaagatgacaaggatgatgatattagtgatactttctctgaaccaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccgagatactttgccaagtaatatgtatcagaaaaatggcttacatcatggaaaatatgcagtaaagaagtcacggagaactgatgtagaagacctgactccaaatcctaaaaaactcctccagataggcaatgaacttcggaaactgaataaggtgattagtgacctgactccagtcagtgagcttcccttaacagcccgaccaaggtcaaggaaggaaaaaaataagctggcttccagagcttgtcggttaaagaagaaagcccagtatgaagctaataaagtgaaattatggggcctcaacacagaatatgataatttattgtttgtaatcaactccatcaagcaagagattgtaaaccgggtacagaatccaagagatgagagaggacccaacatggggcagaagcttgaaatcctcattaaagatactctcggtctaccagttgctgggcaaacctcagaatttgttaaccaagtgttagagaagactgcagaagggaatcccactggaggccttgtaggattaaggataccaacatcaaaggtgtaatcagcctcattggaccactggtcagaaatgtctgcgttttgtcacgttatccattgtaaattttcattctgttttgcatgtcagttagcattatgtaaacatttacaattaggttacattgttttaagaactaagtagcataagtgaagcatgatccaaaatacttgattattgcattttcagagcataaaccatgattaaaactgctactggcatcagaattgaaaatcatatgtttaagtaaatgttaggtacagattacaaaaatctgttaaagcaaaacattttggaggagtgaaatagtaaaatgccaagtattgtggcagatttatgctctgaaccacacaaaaaaattgaggaagcatttttttaaacagtcggtttaaattgtttttagaattattgctttttgttctaattttccacaaccattaatctcacttgtatatggcacacccagcacttgtgcctgtgggccatattagatgttcattgtcagagctcaagatgatatatataaatatatatatatatatatatatatacacacacacacacaaatgtctgtgcaagtaagaaaaaaaaagcatattctttgtgccttgtattttggggaaactctaaaactggtaatattttgtatgatgaaaaccctaatgagaaaaaacaagatatatagatggaaaaattatggggtttaaatgtttttttgttccaactctttttcagattttttgaatgtatataggactatgttgaaatgtagatatatgccacagagtctgtgtattgtataaaaaacaaaacaaaaaacaacaaaaaaaagatggctctagaaaactcatatttcggtacttgaccggaagaagacaaatacttgcacattattgcgattgttttattttttgtaccaaagacaaatgcaactgatatggcaaactgccagtctaagtaaagttttgcacagcttacatgatactgtatgaatgtatgaaaaaaaaggagaaaaaaaagaaaaaaaaaggtcagggttagggatcttactgaactgtgaattttatttctgtttgggtccaattatctacagaaggagcatccatacatacaaatattattttgctgttcctctagttcgcttccatagtagataagttggtggccatttagatgtcttttatttctgcacttattgtaggaaattttaatatatttcattttagtaagctattgataaaatagtttttgactttgaaaattaaaatgtttatttagcttattgtagtatacttccaccagacaacaaaatagattatttttattgtattatgtatatatatatatgtaaagaaagaaaaaagctaaaaatatctaattctttagttgccacttttccgattgatgtattattgtgcatgtaatattttcaaagatcaacacaggctaaaacaaaaacaatttatagatttttatatttttgtacaggtattttcaaactagcttcttcaaacttaacatgtgacttattcttctatagtttctagaattgagaaacattaacacatttagtttttaggtgctcttttttgctcatataaaacagcttcattagtcagtgttttaactgtgttcaagctttacctcttgatgagaaatttcttatgtcaaggcagcattataaaccttcccccacagatttttccatcctgtctctcttactgttttattctcaaatcttgtgctttgaactctgaaaactggtggcttaaaaactaaaaaaagaaaaaaagcatatttagcaaggaaaaaaataccaaaaatttcaggcatagctgctggaaaaattatctatttctccattacccactgtaggatttcttttttaattatactttgactataaagtgtcaaagtataatttgttcttttcttttactttgttaccccatttgtaagctatagcatatgaagctatatatatagcttgtgaaggtttgatctagaacacccagtaacaaatgaacaatgttgcttacctgcttctttgacatcttaaaaaagaaatccaaggaggattgtaaggattgtcttaccaccttagctgaactgtgatgcacaagatttttctatgtgtttggtggaaatgtacctggtttgtacattcacgctaaacagatgataagctcaagtctgatggtttaatagaatgtaagttcatcgtttaaagcttttcctttttaggttggagaaggcaaaacacaggcttgcaagttggaagtatatgaagtcttgacagagtgtgtctggtaaattgaaaagtgtttcaaactatggcagttttgcaatcaggtgaaaatcacctcatgatattcagctgataaggtttataaaattgcccctttctagctgctctgttaggaattctggtttttgatacttttttcctgtctgcaaaccagaatttgattttttggtcttgcatttcaaaaaaaaaaagactttgaatctgtttagtagattccatatctttgagtttcagtgttttatatgtactacttaagttaaatagttaaaagcttttaaatagttgagctttttaatgttgacactttattttgtacctatttatatatgtatgtatatcttagaaaagcactttgttaaaaaaaaattgcattttatatgattcctgccatttgctgctaaatctgggctggtcagaatgctgcagcgatacttgatctatataaaaacctggcagtaaaatgtagagtgaaagttaaatcctcttgctgttttaactttatcataaagatgacataggcaagctgtgcagctttacattttaaccaggggactctgtggcatttaaaaccgtctagaaatggttgtactttaatgccagtaataatctgcttcctctattgtcattaaaatatatacgtttagtgtatcacacaaaccaatcttataagggtaatgtaaaaaccccaacaattgtacatgttctgtttttgaaaattgtggcatgtatttttgggtgaagatcattagagaagagttctctaaaggttttctgtgttcatacatggtatacagatagctcataatgaagtccagaatcttacttttaagtgaaggcattgtgaattcacctcaagtaaacccattgttccaaagcaattataaactttgactctagtactactatgatttaaaaaaaaaaaaaaccaacaaaaaccttttttcctagtttcagatacactggattctttatagagtttgtctccatatgaaagcatgctgtccagtcgctcttgttaagatcttgtctgagttttgaattgggtgccacacttttccagtcaatataattgcttgttctactgtaccatgtatgattcttgtcctttcctatatccttcatgacagattatgatgtggctttatattgtgccttacttgtacatttaaaactaaacgtcttcattcccttccacttcctacatctttaactttgacctttttggtaagagaatcagaactattacaaaagcatcatgaaggatttcagatgggtatggtttcaaattccctctctttatagttattttatatttgtatgaaagaccagttttggatggtctttgaatataggggggaaagattagcagtaatttcactacatcccttttctctgactttcatgcatttctcatacatcttctttctgatgcttgactttatttgcttcctagcaatagtctgcatttaaagaaaggtgtgttcaattcatcagcttgaaattgactatttcatttttccaggattttttaggagaagagtacccattttgttttataaaaacagatgacaagtctctttaaaagaaacagaagtacagtacttttgaaatacaatgctgttagtttggatttctttttatatatatatataatattcatacaatgatctgatgtttgccttcattaataaagctgttagtttattcaccaaaatgtcaagaatggatgtgcttttctttattccacacatttaaaaaaatttagctgctaagatttaatgttataagaaatgaattcaagttgccttcagcaagaattaacaaaaacttatgttccctttctttatatagtttcctaaaattctgttcaagtattttctagttaattatgtaacagaatgttagcatctctccatatcttgaaacttgaattttgagaatgcattgaattatgctttcagtgttaaagtaaaaggtttcaattatccttctagtgaagtctgttgtggaataccatttcccatggaactgaggccatttccacaactttgcacagaactgcagtcttgttcttcccttggatcatgacaaataagtctcacacagtgccgtaatacttgtggattcttttgtaatctttgtaatcttaataagggcattatgagaagacgactccatgtttttttaatacttcaaacacattgggatgtaacaatgaatgtcaactgtaggaatggtggtttcgttttaaggaataagcatgttggggaaagatgatgaaaatgtactactgaaagttatacacttccataggcaaatgggattatgtgttgaagcatagtcctcatgcttaataaactgactgaaatcgtagaaattacacctaggaactgagctaggccaaattgccatttttgtttagagagttttggaggtagtagtgaggggacagagccttaaaactacttccaaacagtattttggaattgaagacttggtaactagtgaagaacatcaaagttgggtatttcaatgtgccaagtttgggtgaactaggttcggtttgcctctttcataacaatgtaaacacaatggtgtagttaattaaattctgggtggataggagcaggactgattactatgtcttgcccttcgccctttgtttttttcagaaccaaataacagaaatgtgtatgtgtgtactgtatctgcctttccaccacatttttatgacactgtattccactgcctgcttttttaccttctttccctaggatttgtcctacagcttagtattgtggttgacagcgatactagggctgacagcacagaagtcacaagagaagagtggaagggcaagaattcaaagcatttgttcatacaatgtggcaacctcttttgcatagttgcgtaggatcctgtttgtaatgctatcataaatattctgtagttttttttttttctctcccaactggagctatgacactttttattggattcagtcttgtctcttgtctagaaagaactttatcttgttgacgcatgagctgtttaaaaattatcctattaaatgttggttaatagttgtgcagtttttcatttcagatggaaaggcaatgcaaattttgcctttgttttctgtcaccttccaacccctgagcacttctagtcagatacagattcatcagtgtatgcaacatcctttgtaatttaaaataaaaaaagatgaaaagaaaacgtt

SEQ ID NO: 6 - DNA, 7782 bp, Artificial Sequence
  Description of Artificial Sequence Synthetic polynucleotide
gagtcacgcgatttccgggaacccgtcaggaaggacataaacaaaacaaacccgaggcagcatggagaggggccgtggcccctgcagcggaaccggacccagtccctgagccgcccctacacccacagacagcatcgcacagaattattttaaaaaaaagcagtgatccaagcaattgaattggaagcactctggggaaacctgctgtttattgtggaaatcatcttcgatcttggaattgaaagtaaagctggaaaggaatttacaaacaagaaaaaaaagaagtttggaatcggattcacaggatctgggcttggaaatgcctcagcctagtgtaagcggaatggatccgcctttcggggatgcctttcgaagccacaccttttcggaacaaactctgatgagcacagatctcttagcaaacagttcggatccagatttcatgtatgaactggatagagagatgaactaccaacagaatcctagagacaactttctttctttggaggactgcaaagacattgaaaatctggagtctttcacagatgtcctggataatgagggtgctttaacctcaaactgggaacagtgggatacatactgtgaagacctaacgaaatataccaaactaaccagctgtgacatctggggaacaaaagaagtggattacttgggtcttgatgacttttctagtccttaccaagatgaagaggttataagtaaaactccaactttagctcaacttaatagtgaggactcacagtctgtttctgattccctttattaccccgattcacttttcagtgtcaaacaaaatcccttaccctcttcattccctggtaaaaagatcacaagcagagcagctgctcctgtgtgttcttctaagactctgcaggctgaggtccctttgtcagactgtgtccaaaaagcaagtaaacccacttcaagcacacaaatcatggtgaagaccaacatgtatcataatgaaaaggtgaactttcatgttgaatgtaaagactatgtaaaaaaggcaaaggtaaagatcaacccagtgcaacagagccggcccttgttgagccagattcacacagatgcagcaaaggagaacacctgctactgtggtgcagtggcaaagagacaagagaaaaaagggatggagcctcttcaaggtcatgccactcccgctttgccttttaaagaaacccaggaactattactaagtcccctgccccaggaaggtcctgggtcacttgcagcaggagagagcagcagtctttctgccagtacatcagtctcagattcatcccagaaaaaagaagagcacaattattctctttttgtctccgacaacttgggtgaacagccaactaaatgcagtcctgaagaagatgaggaggacgaggaggatgttgatgatgaggaccatgatgaaggattcggcagtgagcatgaactgtctgaaaatgaggaggaggaagaagaggaagaggattatgaagatgacaaggatgatgatattagtgatactttctctgaaccaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccaagatactttgccaagtaatatgtatcagaaaaatggcttacatcatggaaaatatgcagtaaagaagtcacggagaactgatgtagaagacctgactccaaatcctaaaaaactcctccagataggcaatgaacttcggaaactgaataaggtgattagtgacctgactccagtcagtgagcttcccttaacagcccgaccaaggtcaaggaaggaaaaaaataagctggcttccagagcttgtcggttaaagaagaaagcccagtatgaagctaataaagtgaaattatggggcctcaacacagaatatgataatttattgtttgtaatcaactccatcaagcaagagattgtaaaccgggtacagaatccaagagatgagagaggacccaacatggggcagaagcttgaaatcctcattaaagatactctcggtctaccagttgctgggcaaacctcagaatttgttaaccaagtgttagagaagactgcagaagggaatcccactggaggccttgtaggattaaggataccaacatcaaaggtgtaatcagcctcattggaccactggtcagaaatgtctgcgttttgtcacgttatccattgtaaattttcattctgttttgcatgtcagttagcattatgtaaacatttacaattaggttacattgttttaagaactaagtagcataagtgaagcatgatccaaaatacttgattattgcattttcagagcataaaccatgattaaaactgctactggcatcagaattgaaaatcatatgtttaagtaaatgttaggtacagattacaaaaatctgttaaagcaaaacattttggaggagtgaaatagtaaaatgccaagtattgtggcagatttatgctctgaaccacacaaaaaaattgaggaagcatttttttaaacagtcggtttaaattgtttttagaattattgctttttgttctaattttccacaaccattaatctcacttgtatatggcacacccagcacttgtgcctgtgggccatattagatgttcattgtcagagctcaagatgatatatataaatatatatatatatatatatatatacacacacacacacaaatgtctgtgcaagtaagaaaaaaaaagcatattctttgtgccttgtattttggggaaactctaaaactggtaatattttgtatgatgaaaaccctaatgagaaaaaacaagatatatagatggaaaaattatggggtttaaatgtttttttgttccaactctttttcagattttttgaatgtatataggactatgttgaaatgtagatatatgccacagagtctgtgtattgtataaaaaacaaaacaaaaaacaacaaaaaaaagatggctctagaaaactcatatttcggtacttgaccggaagaagacaaatacttgcacattattgcgattgttttattttttgtaccaaagacaaatgcaactgatatggcaaactgccagtctaagtaaagttttgcacagcttacatgatactgtatgaatgtatgaaaaaaaaggagaaaaaaaagaaaaaaaaaggtcagggttagggatcttactgaactgtgaattttatttctgtttgggtccaattatctacagaaggagcatccatacatacaaatattattttgctgttcctctagttcgcttccatagtagataagttggtggccatttagatgtcttttatttctgcacttattgtaggaaattttaatatatttcattttagtaagctattgataaaatagtttttgactttgaaaattaaaatgtttatttagcttattgtagtatacttccaccagacaacaaaatagattatttttattgtattatgtatatatatatatgtaaagaaagaaaaaagctaaaaatatctaattctttagttgccacttttccgattgatgtattattgtgcatgtaatattttcaaagatcaacacaggctaaaacaaaaacaatttatagatttttatatttttgtacaggtattttcaaactagcttcttcaaacttaacatgtgacttattcttctatagtttctagaattgagaaacattaacacatttagtttttaggtgctcttttttgctcatataaaacagcttcattagtcagtgttttaactgtgttcaagctttacctcttgatgagaaatttcttatgtcaaggcagcattataaaccttcccccacagatttttccatcctgtctctcttactgttttattctcaaatcttgtgctttgaactctgaaaactggtggcttaaaaactaaaaaaagaaaaaaagcatatttagcaaggaaaaaaataccaaaaatttcaggcatagctgctggaaaaattatctatttctccattacccactgtaggatttcttttttaattatactttgactataaagtgtcaaagtataatttgttcttttcttttactttgttaccccatttgtaagctatagcatatgaagctatatatatagcttgtgaaggtttgatctagaacacccagtaacaaatgaacaatgttgcttacctgcttctttgacatcttaaaaaagaaatccaaggaggattgtaaggattgtcttaccaccttagctgaactgtgatgcacaagatttttctatgtgtttggtggaaatgtacctggtttgtacattcacgctaaacagatgataagctcaagtctgatggtttaatagaatgtaagttcatcgtttaaagcttttcctttttaggttggagaaggcaaaacacaggcttgcaagttggaagtatatgaagtcttgacagagtgtgtctggtaaattgaaaagtgtttcaaactatggcagttttgcaatcaggtgaaaatcacctcatgatattcagctgataaggtttataaaattgcccctttctagctgctctgttaggaattctggtttttgatacttttttcctgtctgcaaaccagaatttgattttttggtcttgcatttcaaaaaaaaaaagactttgaatctgtttagtagattccatatctttgagtttcagtgttttatatgtactacttaagttaaatagttaaaagcttttaaatagttgagctttttaatgttgacactttattttgtacctatttatatatgtatgtatatcttagaaaagcactttgttaaaaaaaaattgcattttatatgattcctgccatttgctgctaaatctgggctggtcagaatgctgcagcgatacttgatctatataaaaacctggcagtaaaatgtagagtgaaagttaaatcctcttgctgttttaactttatcataaagatgacataggcaagctgtgcagctttacattttaaccaggggactctgtggcatttaaaaccgtctagaaatggttgtactttaatgccagtaataatctgcttcctctattgtcattaaaatatatacgtttagtgtatcacacaaaccaatcttataagggtaatgtaaaaaccccaacaattgtacatgttctgtttttgaaaattgtggcatgtatttttgggtgaagatcattagagaagagttctctaaaggttttctgtgttcatacatggtatacagatagctcataatgaagtccagaatcttacttttaagtgaaggcattgtgaattcacctcaagtaaacccattgttccaaagcaattataaactttgactctagtactactatgatttaaaaaaaaaaaaaaccaacaaaaaccttttttcctagtttcagatacactggattctttatagagtttgtctccatatgaaagcatgctgtccagtcgctcttgttaagatcttgtctgagttttgaattgggtgccacacttttccagtcaatataattgcttgttctactgtaccatgtatgattcttgtcctttcctatatccttcatgacagattatgatgtggctttatattgtgccttacttgtacatttaaaactaaacgtcttcattcccttccacttcctacatctttaactttgacctttttggtaagagaatcagaactattacaaaagcatcatgaaggatttcagatgggtatggtttcaaattccctctctttatagttattttatatttgtatgaaagaccagttttggatggtctttgaatataggggggaaagattagcagtaatttcactacatcccttttctctgactttcatgcatttctcatacatcttctttctgatgcttgactttatttgcttcctagcaatagtctgcatttaaagaaaggtgtgttcaattcatcagcttgaaattgactatttcatttttccaggattttttaggagaagagtacccattttgttttataaaaacagatgacaagtctctttaaaagaaacagaagtacagtacttttgaaatacaatgctgttagtttggatttctttttatatatatatataatattcatacaatgatctgatgtttgccttcattaataaagctgttagtttattcaccaaaatgtcaagaatggatgtgcttttctttattccacacatttaaaaaaatttagctgctaagatttaatgttataagaaatgaattcaagttgccttcagcaagaattaacaaaaacttatgttccctttctttatatagtttcctaaaattctgttcaagtattttctagttaattatgtaacagaatgttagcatctctccatatcttgaaacttgaattttgagaatgcattgaattatgctttcagtgttaaagtaaaaggtttcaattatccttctagtgaagtctgttgtggaataccatttcccatggaactgaggccatttccacaactttgcacagaactgcagtcttgttcttcccttggatcatgacaaataagtctcacacagtgccgtaatacttgtggattcttttgtaatctttgtaatcttaataagggcattatgagaagacgactccatgtttttttaatacttcaaacacattgggatgtaacaatgaatgtcaactgtaggaatggtggtttcgttttaaggaataagcatgttggggaaagatgatgaaaatgtactactgaaagttatacacttccataggcaaatgggattatgtgttgaagcatagtcctcatgcttaataaactgactgaaatcgtagaaattacacctaggaactgagctaggccaaattgccatttttgtttagagagttttggaggtagtagtgaggggacagagccttaaaactacttccaaacagtattttggaattgaagacttggtaactagtgaagaacatcaaagttgggtatttcaatgtgccaagtttgggtgaactaggttcggtttgcctctttcataacaatgtaaacacaatggtgtagttaattaaattctgggtggataggagcaggactgattactatgtcttgcccttcgccctttgtttttttcagaaccaaataacagaaatgtgtatgtgtgtactgtatctgcctttccaccacatttttatgacactgtattccactgcctgcttttttaccttctttccctaggatttgtcctacagcttagtattgtggttgacagcgatactagggctgacagcacagaagtcacaagagaagagtggaagggcaagaattcaaagcatttgttcatacaatgtggcaacctcttttgcatagttgcgtaggatcctgtttgtaatgctatcataaatattctgtagttttttttttttctctcccaactggagctatgacactttttattggattcagtcttgtctcttgtctagaaagaactttatcttgttgacgcatgagctgtttaaaaattatcctattaaatgttggttaatagttgtgcagtttttcatttcagatggaaaggcaatgcaaattttgcctttgttttctgtcaccttccaacccctgagcacttctagtcagatacagattcatcagtgtatgcaacatcctttgtaatttaaaataaaaaaagatgaaaagaaaacgtt

SEQ ID NO: 7 - protein, 371 aa, Homo sapiens
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRKIRNKRSAQESRRKKKVYVGGLESRVLKYTAQNMELQNKVQLLEEQNLSLLDQLRKLQAMVIEISNKTSSSSTCILVLLVSFCLLLVPAMYSSDTRGSLPAEHGVLSRQLRALPSEDPYQLELPALQSEVPKDSTHQWLDGSDCVLQAPGNTSCLLHYMPQAPSAEPPLEWPFPDLFSEPLCRGPILPLQANLTRKGGWLPTGSPSVILQDRYSG

SEQ ID NO: 8 - DNA, 1868 bp, Homo sapiens
ggaagcgagggtgcggcgcaatccggagaggacgccaggacgacgcccgagttccctttcaggctagaactcttcctttttctagcttggggtagaaggcggagccggagccccggaacccccgccctcggggtgcgaggcggcagcagggccgtcccctacatttgcatagcccctgggacgtggcgctgcacccaagcctcttctcagttggagggaactccaagtcccacagtgccacggggtggggtgcgtcactttcgctgcgttggaggctgaggagaattgagcctgggaggcgggtccggagagggctatggaaagccgccggcggggaatcccggccgtagagggacagtggataggtgcccgaggcctacagctggcctggggctcgtgtctgggcttcggacgttggggcccggtggcccaccctttccgtagttgtcccaaatggagctggaattggatgctggtgaccaagacctgctggccttcctgctagaggaaagtggagatttggggacggcacccgatgaggccgtgagggccccactggactgggcgctgccgctttctgaggtaccgagcgactgggaagtagatgatttgctgtgctccctgctgagtcccccagcgtcgttgaacattctcagctcctccaacccctgccttgtccaccatgaccacacctactccctcccacgggaaactgtctctatggatctagagagtgagagctgtagaaaagaggggacccagatgactccacagcatatggaggagctggcagagcaggagattgctaggctagtactgacagatgaggagaagagtctattggagaaggaggggcttattctgcctgagacacttcctctcactaagacagaggaacaaattctgaaacgtgtgcggaggaagattcgaaataaaagatctgctcaagagagccgcaggaaaaagaaggtgtatgttgggggtttagagagcagggtcttgaaatacacagcccagaatatggagcttcagaacaaagtacagcttctggaggaacagaatttgtcccttctagatcaactgaggaaactccaggccatggtgattgagatatcaaacaaaaccagcagcagcagcacctgcatcttggtcctactagtctccttctgcctcctccttgtacctgctatgtactcctctgacacaagggggagcctgccagctgagcatggagtgttgtcccgccagcttcgtgccctccccagtgaggacccttaccagctggagctgcctgccctgcagtcagaagtgccgaaagacagcacacaccagtggttggacggctcagactgtgtactccaggcccctggcaacacttcctgcctgctgcattacatgcctcaggctcccagtgcagagcctcccctggagtggccattccctgacctcttctcagagcctctctgccgaggtcccatcctccccctgcaggcaaatctcacaaggaagggaggatggcttcctactggtagcccctctgtcattttgcaggacagatactcaggctagatatgaggatatgtggggggtctcagcaggagcctggggggctccccatctgtgtccaaataaaaagcggtgggcaagggctggccgcagctcctgtgccctgtcaggacgactgagggctcaaacacaccacacttaatggctttctgggtcttttatttgtacccatgtgtctgtcacaccatgaatgtacctggggaaatcaactgacctccctgaacatttcacgcagtcagggaacaggtgaggaaagaaataaataagtgattctaatgctgcctaaaaaaaaaaaaaaaaaa

SEQ ID NO: 9 - DNA, 281 bp, Artificial Sequence
  Description of Artificial Sequence Synthetic polynucleotide
aaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccragatactttgccaagtaatatgtatcagaaaaatggcttacatcatggtaagaggggattgcagtcagatatttagtgtcactttaatcaagttgagctactaatccataatgtttactccgtgtaccta

SEQ ID NO: 10 - DNA, 22 bp, Artificial Sequence
  Description of Artificial Sequence Synthetic primer
caagagaggatgctgagaccat

SEQ ID NO: 11 - DNA, 29 bp, Artificial Sequence
  Description of Artificial Sequence Synthetic primer
accatgatgtaagccatttttctgataca

SEQ ID NO: 12 - DNA, 24 bp, Artificial Sequence
  Description of Artificial Sequence Synthetic primer
atgtatgaactggatagagagatg

SEQ ID NO: 13 - DNA, 24 bp, Artificial Sequence
  Description of Artificial Sequence Synthetic primer
gttaggtcttcacagtatgtatcc

SEQ ID NO: 14 - protein, 6 aa, Artificial Sequence
  Description of Artificial Sequence Synthetic 6xHis tag
HHHHHH

SEQ ID NO: 15 - DNA, 21 bp, Artificial Sequence
  Description of Artificial Sequence Synthetic primer
gaagacctgaaggaggtgact

SEQ ID NO: 16 - DNA, 22 bp, Artificial Sequence
  Description of Artificial Sequence Synthetic primer
gttccactcagatggtctcagc

SEQ ID NO: 17 - DNA, 21 bp, Artificial Sequence
  Description of Artificial Sequence Synthetic primer
gaggacttgaaggagatgacg

SEQ ID NO: 18 - DNA, 20 bp, Artificial Sequence
  Description of Artificial Sequence Synthetic primer
cagaaggcctcagaatcctc

SEQ ID NO: 19 - DNA, 21 bp, Artificial Sequence
  Description of Artificial Sequence Synthetic primer
ccagagcatggtgccttcgct

SEQ ID NO: 20 - DNA, 18 bp, Artificial Sequence
  Description of Artificial Sequence Synthetic primer
cagcaaccattgggtcag

SEQ ID NO: 21 - DNA, 21 bp, Artificial Sequence
  Description of Artificial Sequence Synthetic primer
caagaacagcaacgagtaccg

SEQ ID NO: 22 - DNA, 21 bp, Artificial Sequence
  Description of Artificial Sequence Synthetic primer
gtcactggtcaactccagcac

SEQ ID NO: 23 - DNA, 20 bp, Artificial Sequence
  Description of Artificial Sequence Synthetic primer
ccactgccgcatcctcttcc

SEQ ID NO: 24 - DNA, 24 bp, Artificial Sequence
  Description of Artificial Sequence Synthetic primer
ctcgttgccaatagtgatgacctg

SEQ ID NO: 25 - DNA, 19 bp, Artificial Sequence
  Description of Artificial Sequence Synthetic oligonucleotide
tggttaacaaattctgagg

SEQ ID NO: 26 - DNA, 19 bp, Artificial Sequence
  Description of Artificial Sequence Synthetic oligonucleotide
aggtatctcgattccactc

SEQ ID NO: 27 - DNA, 19 bp, Artificial Sequence
  Description of Artificial Sequence Synthetic oligonucleotide
tggagttttactgatgacc

SEQ ID NO: 28 - DNA, 25 bp, Artificial Sequence
  Description of Artificial Sequence Synthetic oligonucleotide
caccgggattctgaggccttctgag

SEQ ID NO: 29 - DNA, 25 bp, Artificial Sequence
  Description of Artificial Sequence Synthetic oligonucleotide
aaacctcagaaggcctcagaatccc

SEQ ID NO: 30 - DNA, 25 bp, Artificial Sequence
  Description of Artificial Sequence Synthetic oligonucleotide
caccggtatctcgattccactcaga

SEQ ID NO: 31 - DNA, 25 bp, Artificial Sequence
  Description of Artificial Sequence Synthetic oligonucleotide
aaactctgagtggaatcgagatacc

SEQ ID NO: 32 - DNA, 144 bp, Artificial Sequence
  Description of Artificial Sequence Synthetic polynucleotide
gcactaaatatttttcaaacctcttaccatgatgtaagccatttttctggtacatattacttggcaaggtatcttgattccactcagaaggcctcagaatcctctcttgctgtgatggtgtaagctgctcactatactcccaga

SEQ ID NO: 33 - DNA, 20 bp, Artificial Sequence
  Description of Artificial Sequence Synthetic primer
ccgctaatgcttctgtagcc

SEQ ID NO: 34 - DNA, 20 bp, Artificial Sequence
  Description of Artificial Sequence Synthetic primer
gattacccgaggcagttgag

SEQ ID NO: 35 - DNA, 20 bp, Artificial Sequence
  Description of Artificial Sequence Synthetic primer
tggaagctgctctgctatcg

SEQ ID NO: 36 - DNA, 20 bp, Artificial Sequence
  Description of Artificial Sequence Synthetic primer
aagtcccatccacattgctc

SEQ ID NO: 37 - DNA, 20 bp, Artificial Sequence
  Description of Artificial Sequence Synthetic primer
ggagatggatgacagcaagg

SEQ ID NO: 38 - DNA, 20 bp, Artificial Sequence
  Description of Artificial Sequence Synthetic primer
ggacatgaggcacactggta

SEQ ID NO: 39 - DNA, 20 bp, Artificial Sequence
  Description of Artificial Sequence Synthetic primer
gacaggcacttctcccagag

SEQ ID NO: 40 - DNA, 20 bp, Artificial Sequence
  Description of Artificial Sequence Synthetic primer
tcaagggcatagagcagtcc

SEQ ID NO: 41 - DNA, 20 bp, Artificial Sequence
  Description of Artificial Sequence Synthetic primer
cccgtaagaagcgaagtctg

SEQ ID NO: 42 - DNA, 20 bp, Artificial Sequence
  Description of Artificial Sequence Synthetic primer
cattgagcctgagctgtgaa

SEQ ID NO: 43 - DNA, 17 bp, Unknown
  Description of Unknown Wild type Crebrf sequence
caaggtatctcgattcc

SEQ ID NO: 44 - DNA, 17 bp, Artificial Sequence
  Description of Artificial Sequence Synthetic oligonucleotide
caatgtatcttgattcc

SEQ ID NO: 45 - DNA, 96 bp, Artificial Sequence
  Description of Artificial Sequence Synthetic oligonucleotide
ttacaccatcacagcaagagaggattctgaggccttctgagtggaatcgagataccttgccaagtaatatgtaccagaaaaatggcttacatcatg

SEQ ID NO: 46 - DNA, 23 bp, Artificial Sequence
  Description of Artificial Sequence Synthetic primer
ctggtacatattacttggcaagg

SEQ ID NO: 47 - DNA, 23 bp, Artificial Sequence
  Description of Artificial Sequence Synthetic primer
ggattctgaggccttctgagtgg

SEQ ID NO: 48 - DNA, 23 bp, Artificial Sequence
  Description of Artificial Sequence Synthetic primer
tttttctggtacatattacttgg

SEQ ID NO: 49 - DNA, 64 bp, Artificial Sequence
  Description of Artificial Sequence Synthetic primer
  modified_base (25)..(44): a, c, t, g, unknown or other
gaaattaatacgactcactataggnnnnnnnnnnnnnnnnnnnngttttagagctagaaatagc

SEQ ID NO: 50 - DNA, 64 bp, Artificial Sequence
  Description of Artificial Sequence Synthetic oligonucleotide
gaaattaatacgactcactataggctggtacatattacttggcagttttagagctagaaatagc

SEQ ID NO: 51 - DNA, 67 bp, Artificial Sequence
  Description of Artificial Sequence Synthetic oligonucleotide
gaaattaatacgactcactataggggattctgaggccttctgagtgggttttagagctagaaatagc

SEQ ID NO: 52 - DNA, 20 bp, Artificial Sequence
  Description of Artificial Sequence Synthetic primer
tttaatgcctggcaccattt

SEQ ID NO: 53 - DNA, 20 bp, Artificial Sequence
  Description of Artificial Sequence Synthetic primer
tgacaattgtgggaccatgt

SEQ ID NO: 54 - DNA, 20 bp, Artificial Sequence
  Description of Artificial Sequence Synthetic primer
gaacgaggcagaggattcaa

SEQ ID NO: 55 - DNA, 20 bp, Artificial Sequence
  Description of Artificial Sequence Synthetic primer
agaaggagccgttgtgacag

SEQ ID NO: 56 - DNA, 20 bp, Artificial Sequence
  Description of Artificial Sequence Synthetic primer
ccacactgatggaagctgtg

SEQ ID NO: 57 - DNA, 25 bp, Artificial Sequence
  Description of Artificial Sequence Synthetic primer
aaagaaggtacttctgggagtatag

SEQ ID NO: 58 - DNA, 22 bp, Artificial Sequence
  Description of Artificial Sequence Synthetic probe
agcagcttacaccatcacagca

SEQ ID NO: 59 - DNA, 22 bp, Artificial Sequence
  Description of Artificial Sequence Synthetic primer
caaagagacttagaggccagtc

SEQ ID NO: 60 - DNA, 12 bp, Artificial Sequence
  Description of Artificial Sequence Synthetic probe
accttgccaagt

SEQ ID NO: 61 - DNA, 12 bp, Artificial Sequence
  Description of Artificial Sequence Synthetic probe
ccttctgagtgg

SEQ ID NO: 62 - DNA, 330 bp, Artificial Sequence
  Description of Artificial Sequence Synthetic polynucleotide
aaaatgactctgtagaggacttgaaggagatgacgtccatatcttctcggaagagagggaaaagaaggtacttctgggagtatagtgagcagcttacaccatcacagcaagagaggattctgaggccttctgagtggaatcgagataccttgccaagtaatatgtaccagaaaaatggcttacatcatggtaagaggtttgaaaaatatttagtgcccttttctcttttttttagggggtgaaggtactttttattttgagtagatagcctagactggcctctaagtctctttgtagcccagtttggctttgaactttgaatcctctgcc

SEQ ID NO: 63 - DNA, 7572 bp, Artificial Sequence
  Description of Artificial Sequence Synthetic polynucleotide
gagcagaaactcatctctgaagaggatctgcatcaccatcaccatcactagtgatccgttcaactagcagaccattatcaacaaaatactccaattggcgatggccctgtccttttaccagacaaccattacctgtcgacacaatctgccctttccaaagatcccaacgaaaagcgtgaccacatggtccttcttgagtttgtaactgctgctgggattacacatggcatggatgagctctacaaataatgaattaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgatcaaacaagtttgtacaaaaaagcaggcttaaggagtttaaacaccatgcctcagcctagtgtaagcggaatggatccgcctttcggggatgcctttcgaagccacaccttttcggaacaaactctgatgagcacagatctcttagcaaacagttcggatccagatttcatgtatgaactggatagagagatgaactaccaacagaatcctagagacaactttctttctttggaggactgcaaagacattgaaaatctggagtctttcacagatgtcctggataatgagggtgctttaacctcaaactgggaacagtgggatacatactgtgaagacctaacgaaatataccaaactaaccagctgtgacatctggggaacaaaagaagtggattacttgggtcttgatgacttttctagtccttaccaagatgaagaggttataagtaaaactccaactttagctcaacttaatagtgaggactcacagtctgtttctgattccctttattaccccgattcacttttcagtgtcaaacaaaatcccttaccctcttcattccctggtaaaaagatcacaagcagagcagctgctcctgtgtgttcttctaagactctgcaggctgaggtccctttgtcagactgtgtccaaaaagcaagtaaacccacttcaagcacacaaatcatggtgaagaccaacatgtatcataatgaaaaggtgaactttcatgttgaatgtaaagactatgtaaaaaaggcaaaggtaaagatcaacccagtgcaacagagccggcccttgttgagccagattcacacagatgcagcaaaggagaacacctgctactgtggtgcagtggcaaagagacaagagaaaaaagggatggagcctcttcaaggtcatgccactcccgctttgccttttaaagaaacccaggaactattactaagtcccctgccccaggaaggtcctgggtcacttgcagcaggagagagcagcagtctttctgccagtacatcagtctcagattcatcccagaaaaaagaagagcacaattattctctttttgtctccgacaacttgggtgaacagccaactaaatgcagtcctgaagaagatgaggaggacgaggaggatgttgatgatgaggaccatgatgaaggattcggcagtgagcatgaactgtctgaaaatgaggaggaggaagaagaggaagaggattatgaagatgacaaggatgatgatattagtgatactttctctgaaccaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccgagatactttgccaagtaatatgtatcagaaaaatggcttacatcatggaaaatatgcagtaaagaagtcacggagaactgatgtagaagacctgactccaaatcctaaaaaactcctccagataggcaatgaacttcggaaactgaataaggtgattagtgacctgactccagtcagtgagcttcccttaacagcccgaccaaggtcaaggaaggaaaaaaataagctggcttccagagcttgtcggttaaagaagaaagcccagtatgaagctaataaagtgaaattatggggcctcaacacagaatatgataatttattgtttgtaatcaactccatcaagcaagagattgtaaaccgggtacagaatccaagagatgagagaggacccaacatggggcagaagcttgaaatcctcattaaagatactctcggtctaccagttgctgggcaaacctcagaatttgttaaccaagtgttagagaagactgcagaagggaatcccactggaggccttgtaggattaaggataccaacatcaaaggtgtacctcgagtgcggccgc

SEQ ID NO: 64 - DNA, 7571 bp, Artificial Sequence
  Description of Artificial Sequence Synthetic polynucleotide
gagcagaaactcatctctgaagaggatctgcatcaccatcaccatcactagtgatccgttcaactagcagaccattatcaacaaaatactccaattggcgatggccctgtccttttaccagacaaccattacctgtcgacacaatctgccctttccaaagatcccaacgaaaagcgtgaccacatggtccttcttgagtttgtaactgctgctgggattacacatggcatggatgagctctacaaataatgaattaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgatcaaacaagtttgtacaaaaaagcaggcttaaggagtttaaacaccatgcctcagcctagtgtaagcggaatggatccgcctttcggggatgcctttcgaagccacaccttttcggaacaaactctgatgagcacagatctcttagcaaacagttcggatccagatttcatgtatgaactggatagagagatgaactaccaacagaatcctagagacaactttctttctttggaggactgcaaagacattgaaaatctggagtctttcacagatgtcctggataatgagggtgctttaacctcaaactgggaacagtgggatacatactgtgaagacctaacgaaatataccaaactaaccagctgtgacatctggggaacaaaagaagtggattacttgggtcttgatgacttttctagtccttaccaagatgaagaggttataagtaaaactccaactttagctcaacttaatagtgaggactcacagtctgtttctgattccctttattaccccgattcacttttcagtgtcaaacaaaatcccttaccctcttcattccctggtaaaaagatcacaagcagagcagctgctcctgtgtgttcttctaagactctgcaggctgaggtccctttgtcagactgtgtccaaaaagcaagtaaacccacttcaagcacacaaatcatggtgaagaccaacatgtatcataatgaaaaggtgaactttcatgttgaatgtaaagactatgtaaaaaaggcaaaggtaaagatcaacccagtgcaacagagccggcccttgttgagccagattcacacagatgcagcaaaggagaacacctgctactgtggtgcagtggcaaagagacaagagaaaaaagggatggagcctcttcaaggtcatgccactcccgctttgccttttaaagaaacccaggaactattactaagtcccctgccccaggaaggtcctgggtcacttgcagcaggagagagcagcagtctttctgccagtacatcagtctcagattcatcccagaaaaaagaagagcacaattattctctttttgtctccgacaacttgggtgaacagccaactaaatgcagtcctgaagaagatgaggaggacgaggaggatgttgatgatgaggaccatgatgaaggattcggcagtgagcatgaactgtctgaaaatgaggaggaggaagaagaggaagaggattatgaagatgacaaggatgatgatattagtgatactttctctgaaccaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccaagatactttgccaagtaatatgtatcagaaaaatggcttacatcatggaaaatatgcagtaaagaagtcacggagaactgatgtagaagacctgactccaaatcctaaaaaactcctccagataggcaatgaacttcggaaactgaataaggtgattagtgacctgactccagtcagtgagcttcccttaacagcccgaccaaggtcaaggaaggaaaaaaataagctggcttccagagcttgtcggttaaagaagaaagcccagtatgaagctaataaagtgaaattatggggcctcaacacagaatatgataatttattgtttgtaatcaactccatcaagcaagagattgtaaaccgggtacagaatccaagagatgagagaggacccaacatggggcagaagcttgaaatcctcattaaagatactctcggtctaccagttgctgggcaaacctcagaatttgttaaccaagtgttagagaagactgcagaagggaatcccactggaggccttgtaggattaaggataccaacatcaaaggtgtacctcgagtgcggccg

(19) United States
(12) Patent Application Publication
(10) Pub. No.: US 2018/0245155 A1
(43) Pub. Date: Aug. 30, 2018

(54) Compositions and Methods for Identifying Genetic Predisposition to Obesity and for Enhancing Adipogenesis

(71) Applicants: Stephen T. MCGARVEY, Providence, RI (US); Ranjan DEKA, Mason, OH (US); Nicola L. HAWLEY, New Haven, CT (US); Ryan Lee MINSTER, Pittsburgh, PA (US); Zsolt URBAN, Pittsburgh, PA (US); Chi-Ting SU, Pittsburgh, PA (US); Erin Elizabeth KERSHAW, Pittsburgh, PA (US); Brown University, Providence, RI (US); University of Cincinnati, Cincinnati, OH (US); University of Pittsburgh - Of the Commonwealth System of Higher Education, Pittsburgh, PA (US)

(72) Inventors: Stephen T. McGarvey, Providence, RI (US); Daniel E. Weeks, Pittsburgh, PA (US); Ranjan Deka, Mason, OH (US); Nicola L. Hawley, New Haven, CT (US); Ryan Lee Minster, Pittsburgh, PA (US); Zsolt Urban, Pittsburgh, PA (US); Chi-Ting Su, Pittsburgh, PA (US); Erin Elizabeth Kershaw, Pittsburgh, PA (US)

(21) Appl. No.: 15/752,687

(22) PCT Filed: Sep. 3, 2016
(86) PCT No.: PCT/US16/50304

§ 371 Date: Feb. 14, 2018

Related U.S. Application Data
(60) Provisional application No. 62/214,045, filed on Sep. 3, 2015.

(51) Int. Cl. C12Q 1/6883

(52) U.S. Cl. CPC C12Q 1/6883; C12Q 2600/158; C12Q 2600/156; C12Q 2600/136

(57) ABSTRACT

The present invention provides compositions and methods for identifying a subject as having a genetic predisposition to obesity or at risk of developing obesity. The present invention also provides compositions and methods for expressing a CREBRF polypeptide of the invention in a cell or precursor thereof and cells expressing a nucleic acid molecule encoding a CREBRF polypeptide of the invention.

### CROSS REFERENCE TO RELATED APPLICATIONS

This application claims priority to U.S. provisional patent application No. 62/214,045, filed Sep. 3, 2015, the content of which is incorporated herein by reference in its entirety.

### STATEMENT OF RIGHTS TO INVENTIONS MADE UNDER FEDERALLY SPONSORED RESEARCH

This invention was made with government support under Grant Nos. R01-HL093093, RO1-AG009375, R01-DK059642, R01-HL090648, R01-DK055406, R01-HL052611, R01-DK090166 and P30 ES006096 awarded by the National Institutes of Health. The government has certain rights in the invention.

### BACKGROUND OF THE INVENTION

Obesity is a condition characterized by the accumulation of excess body fat, and is linked to a negative effect on health. In general, accumulation of body fat is a physiological consequence when caloric intake exceeds an individual's physiological energy requirements. However, the underlying mechanisms of obesity are postulated to result from an interplay of both environmental and genetic factors. In 1962 James Neel posited the existence of a “thrifty gene” that enhances survival in times of famine and promotes metabolic diseases in times of nutritional excess. Thus, under certain dietary conditions, genes controlling appetite and metabolism may predispose an individual to obesity. Indeed, the abundant availability of food in recent times, especially energy-rich and processed foods, has been accompanied by a sharp increase in the prevalence of obesity, revealing pre-existing genetic predispositions to the efficient generation and storage of body fat.

Compared to most populations worldwide, obesity is much more prevalent in Samoans. This high prevalence is likely due to a combination of at least three influences: (i) changes in dietary and physical activity in a small population exposed to economic development, (ii) historical selective pressures enriching for energy-efficient genetic variants that promote survival in times of starvation but obesity in the modern context of low physical activity and caloric excess, (iii) and genetic divergence from other populations due to founder effects, geographic isolation, and population bottlenecks. As a result, Samoans represent a unique population for identifying novel genetic factors contributing to obesity.

Obesity is essentially a disorder of energy homeostasis (i.e., an imbalance between intake and expenditure) and has strong genetic and environmental components. Indeed, as diets have modernized and physical activity has decreased, rates of overweight and obesity in the Samoan population have escalated to among the highest in the world. In 2003, 84% of women and 68% of men in Samoa were overweight or obese by Polynesian cutoffs (BMI>26 kg/m²); in 2010, the prevalence had increased to 91% and 80%, respectively. Although environmental contributors to this trend are clear, the estimated 45% heritability of BMI in this population remains largely unexplained. Samoan genetic susceptibility to obesity in the contemporary obesogenic environment may have resulted from putative advantages of efficient metabolism during 3,000 years of island discoveries, settlement, and population dynamics and/or from genetic drift due to founder effects, small population sizes, and population bottlenecks.

Although obesity is a serious health concern, obesity is a preventable cause of death. The ability to assess an individual's genetic predisposition to obesity would allow for early intervention and treatment. Additionally, understanding molecular mechanisms regulating metabolic efficiency has the potential to provide new therapies for obesity and metabolic disorders. To address these unmet needs, new methods of assessing and characterizing obesity are urgently required.

### SUMMARY OF THE INVENTION

The present invention provides compositions and methods for identifying a subject as having a genetic predisposition to obesity or at risk of developing obesity (e.g., BMI>30 kg/m²). The present invention also provides compositions and methods for expressing a CREBRF polypeptide of the invention in an adipocyte or precursor thereof and cells expressing a nucleic acid molecule encoding a CREBRF polypeptide of the invention.

Thus, in one aspect, the invention provides a recombinant cell comprising a promoter operably linked to a nucleic acid sequence encoding a CREB3 Regulatory Factor (CREBRF) polypeptide.

In an embodiment of this aspect of the invention, the cell is selected from the group consisting of a preadipocyte, an adipocyte, an hepatocyte and precursors thereof. In one embodiment, cell is an adipocyte and is differentiated from a 3T3-L1 cell. In a related embodiment, the promoter is an adipocyte specific promoter. In another embodiment, the cell is an hepatocyte. In one embodiment, the cell hepatic cell is an HepG2 cell. In a related embodiment, the promoter is an hepatocyte specific promoter.

In one embodiment, the nucleic acid comprises a nucleic acid sequence set forth in FIG. 21 or FIG. 22. In another embodiment, the nucleic acid encodes a mutant murine CREBRF polypeptide or a human CREBRF peptide. In a related embodiment, the mutant CREBRF polypeptide comprises a substitution Arg457Gln. In another related embodiment, the nucleic acid comprises a deletion of exon 5 of CREBRF gene. In yet another related embodiment, the mutation is in the cell endogenous CREBRF locus.

In another embodiment, the nucleic acid reduces or eliminates the expression of CREBRF polypeptide. In a further embodiment, the nucleic acid encodes an inhibitory RNA against the CREBRF mRNA. In yet another embodiment, the nucleic acid encodes a shRNA against the CREBRF mRNA.

In one embodiment, the remcombinant cell comprises a CRISPR/Cas9 vector having the nucleic acid sequence that targets the CREBRF gene. In another embodiment, the nucleic acid sequence guides the deletion of the exon 5 of the CREBRF gene. In yet another embodiment, the nucleic acid sequence guides the substitution of arginine at position 457 or its equivalent by glutamine.

Another aspect of the invention provides an expression vector comprising a promoter operably linked to a nucleic acid sequence encoding a CREBRF polypeptide. In one embodiment, the CREBRF polypeptide comprises a glutamine at amino acid position 457. In another embodiment, the expression vector comprises a nucleic acid sequence set forth in FIG. 21 or FIG. 22.

In one embodiment, expression of the nucleic acid reduces or eliminates the expression of CREBRF polypeptide. In another embodiment, the nucleic acid encodes a shRNA against the CREBRF mRNA.

In another aspect, the invention provides an expression vector comprising a CRISP/Cas9 module operably linked to a nucleic acid targeting against a CREBRF gene, wherein the nucleic acid guides the deletion of the exon 5 of the CREBRF gene. In one embodiment, the nucleic acid guides the substitution of arginine at position 457 or its equivalent by glutamine.

Yet another aspect, the invention provides a recombinant cell comprising the expression vector of the aspects and associated embodiments heretofore described.

In another aspect, the invention provides a nucleic acid probe that specifically binds a nucleic acid encoding a CREB3 Regulatory Factor (CREBRF) polypeptide comprising a glutamine at amino acid position 457. In one embodiment, the nucleic acid probe further comprises a detectable label. In another embodiment, the nucleic acid probe is a TaqMan® probe. In yet another embodiment, the nucleic acid probe comprises the nucleic acid sequence:

5′-AGTGGAACCGAGATAC-3′ or 5′-AGTGGAACCAAGATAC-
3′.

Another aspect of the invention provides a knock-in mouse comprising a nucleic acid encoding a mutant murine CREB3 Regulatory Factor (CREBRF) polypeptide or a human CREBRF polypeptide. In one embodiment, the mutant CREBRF polypeptide comprise a substitution Arg457Gln or its equivalent. In a related embodiment, the substitution Arg457Gln or its equivalent is in the mouse endogenous CREBRF locus.

In one embodiment, the mouse is a wild type mouse. In another embodiment, the mutation confers thriftiness to the mouse.

The invention also provides a variety of methods that make use of any of the various embodiments of any aspect delineated herein.

Thus, in one aspect, the invention provides a method of enhancing adipogenesis in a cell, increasing lipid accumulation in a cell or of making a cell resistant to starvation, the method comprising causing the cell to express or overexpress a CREB3 Regulatory Factor (CREBRF) polypeptide. In one embodiment, the CREBRF polypeptide comprises a glutamine at amino acid position 457. In another embodiment, the cell is selected from the group consisting of a preadipocyte, an adipocyte, an hepatocyte and precursors thereof. In one embodiment, cell is an adipocyte and is differentiated from a 3T3-L1 cell. In another embodiment, the cell is an hepatocyte. In a related embodiment, the cell hepatic cell is an HepG2 cell. In yet another embodiment, the cell is in a human subject.

Another aspect of the invention provides method of genotyping a subject comprising contacting a cell of the subject with a nucleic acid probe described herein above. In one embodiment, the method further comprises obtaining the nucleic acid probe described herein above.

In yet another embodiment, the invention provides a method of identifying a subject as obese or at risk of obesity, the method comprising detecting one or more alleles encoding a CREB3 Regulatory Factor (CREBRF) polypeptide comprising a glutamine at amino acid position 457 in a biological sample from the subject, wherein the presence of one or more alleles encoding a CREBRF polypeptide comprising a glutamine at amino acid position 457 indicates that the subject is obese or is at risk of obesity.

In a related aspect, the invention provides a method of treating a subject identified as being obese or at risk of obesity, the method comprising administering the said identified subject a therapeutically effective amount of a compound that modulates adipogenesis in a cell of said subject, wherein the subject is identified as being obese or being at risk of obesity, the method comprising detecting one or more alleles encoding a CREB3 Regulatory Factor (CREBRF) polypeptide comprising a glutamine at amino acid position 457 in a biological sample from the subject, wherein the presence of one or more alleles encoding a CREBRF polypeptide comprising a glutamine at amino acid position 457 indicates that the subject is obese or is at risk of obesity.

In one embodiment of the foregoing methods, the allele comprises an A at position 1689 of a CREBRF polynucleotide. In another embodiment, the subject is human.

Another aspect of the invention provides a method of reducing adipogenesis or lipid accumulation in a cell, the method comprising reducing, eliminating or inactivating the adipogenic function of a CREBRF polypeptide in the cell.

In a related aspect, the invention provides a method of making a cell susceptible to starvation, the method comprising reducing, eliminating or inactivating the adipogenic function of a CREB3 Regulatory Factor (CREBRF) polypeptide.

In an embodiment of these methods, exon 5 of a CREBRF gene is deleted from the cell endogenous CREBRF locus. In one embodiment, the cell is selected from the group consisting of a preadipocyte, an adipocyte, an hepatocyte and precursors thereof. In another embodiment, the cell is an adipocyte and is differentiated from a 3T3-L1 cell. In yet another embodiment, the cell is an hepatocyte. In one embodiment, the cell hepatic cell is an HepG2 cell. In still another embodiment, the cell is a cell of a human subject.

Another aspect of the invention provides a method of identifying a compound that modulates the expression of a CREB3 Regulatory Factor (CREBRF) polypeptide, comprising:

a) contacting a nucleic acid that expresses a CREBRF polypeptide with a compound under conditions suitable for expression by the nucleic acid;

b) determining the level of expression of the CREBRF polypeptide;

c) determining the level of expression of the nucleic acid in the absence of the compound; and

d) comparing the level of expression of the nucleic acid after contact with the compound with the level of expression of the nucleic acid without contact of the compound;

thereby identifying a compound that modulates expression of the CREBRF polypeptide. In one embodiment, the compound is contacted with a recombinant cell or a knock-in mouse as heretofore described herein. In one embodiment, the nucleic acid comprises a nucleic acid sequence set forth in FIG. 21 or FIG. 22. In another embodiment, the CREBRF polypeptide comprises a glutamine at amino acid position 457.

In a related aspect, the invention provides a method of identifying a compound that modulates adipogenesis, the method comprising contacting a recombinant cell or a knock-in mouse, as heretofore described herein, with a compound, and assaying reporter expression in the contacted cell relative to a corresponding control cell, thereby identifying a compound that modulates adipogenesis.

Another related aspect of the invention provides a method of identifying a compound that modulate adipogenesis, the method comprising contacting a recombinant cell or a knock-in mouse, as heretofore described herein, with an shRNA against a gene of interest, and analyzing adipogenesis of the cell relative to a reference, thereby identifying an adipogenesis modulator.

In one embodiment, the adipogenesis of the cell is analyzed by detecting the amplitude, period length and phase of reporter expression. In another embodiment, the reference is an untreated control cell.

In an embodiment, the compound that modulates adipogenesis is an inhibitory nucleic acid molecule, a small organic molecule, or a polypeptide. In a related embodiment, the inhibitory nucleic acid molecule is an shRNA. In another embodiment, the methods further comprises obtaining the recombinant cell or the knock-in mouse described herein above.

In another aspect, the invention provides a kit comprising an expression vector described hierein above and instuctions for use. In a related aspect, the invention provides a kit comprising a nucleic acid probe described hierein above and instuctions for use. In yet another related aspect, the invention provides a kit comprising a knock-in mouse described hierein above and instuctions for use.

In various embodiments of the kits provided by the invention, the instructions for use are for use in accordance with any of the methods described hereinabove.

Other features and advantages of the invention will be apparent from the detailed description, and from the claims.

### BRIEF DESCRIPTION OF THE DRAWINGS

FIGS. 1A and 1B depict principal components analyses. FIG. 1A depicts a scatter plot of the first three principal components from the principal components analysis of the Samoan and HapMap Phase 3 populations. Continental population abbreviations: SAM, Samoans (n=250); EUR, Europeans (n=253); AFR, Africans (n=511); EAS, East Asians (n=255); SAS, South Asians (n=88); AMR, admixed Americans (n=77). FIG. 1B depicts scatter plots of the first six principal components from the principal components analysis of the Samoans alone (n=3,094) plotted against each other.

FIG. 2 depicts a quantile-quantile (QQ) plot for body mass index (BMI). A quantile-quantile plot of the observed −log 10(P-values) for association of BMI in the discovery sample versus the −log 10(P-value) as expected under no association.

FIGS. 3A-3C depict the results of a genome-wide association study (GWAS), targeted sequencing results, and beanplots of BMI versus genotype and gender. FIG. 3A depicts a Manhattan plot of the genome-wide association scan for association with BMI a strong association with BMI at rs12513649 (P=5.3×10⁻¹⁴) on chromosome 5q35.1. FIG. 3B depicts association results using the imputed data within the region of CREBRF, drawn using LocusZoom²⁰. The strength of linkage disequilibrium, as measured by the squared correlation of the genotype dosages, between each SNP and the missense variant rs373863828 is indicated by the coloring of each point. FIG. 3C depicts beanplots of BMI versus genotype in males and females in the discovery sample. Each bean consists of a mirrored density curve containing a 1-D scatterplot of the individual data. The heavy dark line shows the average within each group, while the dotted line indicates the overall average. Drawn using the R ‘beanplot’ package.

FIG. 4 depicts the conditional associations of targeted sequencing genotypes with body mass index. Associations between SNPs in the targeted sequencing regions and body mass index are conditioned on (a) rs12513649, (b) rs150207780, (c) rs373863828, and (d) rs3095870. The red line in each plot corresponds to a P value of 5×10-8. n=3,072 Samoans.

FIG. 5 depicts the beanplots of body mass index in GWAS and replication samples stratified by missense variant rs373863828 genotype, sex and nation. Each bean consists of a mirrored density curve containing a 1-D scatterplot of the individual data. The heavy dark line shows the average within each group, while the dotted line indicates the overall average. Drawn using the R ‘beanplot’ package33. Sample sizes are indicated in [Supplemental Table 1].

FIGS. 6A and 6B depict expression of CREBRF in human and murine tissues. FIG. 6A is a graph depicting human CREBRF mRNA expression in multiple human tissues using Human cDNA Arrays from Origene. FIG. 6B is a graph depicting murine Crebif mRNA expression in murine tissues obtained from 10 week-old, ad lib-fed, male C56BL/6J mice (n=6-12/group). Expression was normalized to the endogenous control gene peptidylprolyl isomerase A/cyclophilin A (PPIA for human; Ppia for mouse). Values represent relative expression and are expressed as mean plus s.e.m. No statistical comparisons were performed. Abbreviations: pg, perigonadal; sc, inguinal subcutaneous; mes, mesenteric. These data support the presence/absence of CREBRF in specific tissues but should be used with caution when assessing relative expression, particularly in humans where precise conditions at the time of tissue collection are not known. Gene expression can be compared to additional in silico resources including BGTEx portal (http://www.gtexportal.org/home/gene/CREBRF) and the BioGPS portal (http://biogps.org/).

FIG. 7 depicts expression of mouse CREBRF relative to key adipogenic genes during adipocyte differentiation. 3T3-L1 cells were treated with a hormonal differentiation cocktail at 2 days post-confluence (day 0, DO) and RNA samples were collected at the indicated time points. mRNA expression relative to the beta actin (Actb) reference gene were determined using quantitative real-time PCR with expression with DO values set at 1. Values are means±SEM of 8 replicates.

FIGS. 8A-8I depict characterization of CREBRF variants with regard to adipogenic differentiation lipid accumulation and energy homeostasis. 3T3-L1 preadipocytes overexpressing enhanced GFP-only negative control (eGFP), wild-type human CREBRF (WT), or the p.Arg457Gln variant human CREBRF were collected at 8 days post-confluence in the absence of hormonal stimulation of adipogenic differentiation. FIGS. 8A-8E are graphs depicting expression of human CREBRF (8A), mouse Crebrf (8B), Pparg2 (8C), Cebpa (8D) and Adipoq (8E) (adiponectin) mRNA, respectively, relative to the beta actin (Actb) reference gene using quantitative real-time PCR. Values are means±SEM of 3 biological and 4 technical replicates (n=3×4=12). Representative results of one of 4 experiments are shown. FIG. 8F is a graph depicting quantification of Oil red O staining by optical density normalized to protein content (OD560/ug protein). Data are means±SEM for 24 replicates each: 3 transfection replicates for each construct and 8 wells for each transfection (n=3×8=24). FIG. 8G depicts representative photomicrographs of oil red O to visualize lipid droplets (red) and hematoxylin (blue) to counterstain nuclei. Scale bar: 50 μm. FIG. 8H depicts Biochemical assay of triglycerides. Data are means±s.e.m., n=2. FIG. 8I are graphs depicting key cellular bioenergenic variables as determined based on real-time oxygen consumption rate (OCR) and extracellular acidification rate (ECAR) normalized to protein content. Values are means±SEM of 6 replicates (n=6). Statistical analysis: one-way analysis of variance (ANOVA), two-sided Games-Howell post hoc test. *P<0.03; **P<10⁻³; ***P<10⁻⁴ compared to 3T3-L1 transfected with eGFP control. ^{#}P<0.05 compared to 3T3-L1 transfected with WT CREBRF.

FIG. 9 presents graphs depicting bioenergetic profile changes during adipocyte differentiation. 3T3-L1 cells were treated with a differentiation cocktail at 2 days post-confluence (day 0, DO), and bioenergetic variables were determined by based on oxygen consumption rate (OCR) and extracellular acidification rate (ECAR) measurements normalized to to protein content. Values are means±SEM (n=6). *: p<0.01 compared to DO (two-tailed t test with unequal variances). As the results were consistent with previously published data24,25, the experiment was performed once.

FIGS. 10A-10D show that Crebrf is induced by nutritional stress and protects against starvation-induced cell death. FIG. 10A is a graph depicting expression of Crebrf mRNA in 3T3-L1 preadipocytes treated with Hank's balanced salt solution (HBSS, starvation) for 0, 2, 4, 12, or 24 hours. Cells were collected at the indicated time points. An additional set of cells subjected to starvation for 12 hours were then “refed” by culturing in fresh growth medium for a subsequent 12 hours (“24 hR”). Expression of Crebrf mRNA relative to beta actin (Actb) reference was then determined by quantitative real-time PCR and normalized to baseline expression (“0h”). Values are means±SEM of 3 biological and 4 technical replicates (n=3×4=12). The red dotted line indicates a relative expression value of 5 for comparison across treatments. Statistical analysis was performed using one way ANOVA and two-sided Bonferroni post-hoc tests. **P=0.002; ***P<1×10⁻¹¹ compared to cells without starvation; ^{###}P=8.8×10⁻¹³ compared to 24 hR. FIG. 10B is a graph depicting expression of Crebrf mRNA in 3T3-L1 preadipocytes treated with 20 ng/ml rapamycin for 0, 2, 4, 12, or 24 hours. Cells were collected at the indicated time points. An additional set of cells subjected to rapamycin treatment for 12 hours were then “refed” by culturing in fresh growth medium for a subsequent 12 hours (“24 hR”). Expression of Crebrf mRNA relative to beta actin (Actb) reference was then determined by quantitative real-time PCR and normalized to baseline expression (“0h”). Values are means±SEM of 3 biological and 4 technical replicates (n=3×4=12). The red dotted line indicates a relative expression value of 5 for comparison across treatments. Statistical analysis was performed using one way ANOVA and two-sided Bonferroni post-hoc tests. ***P<1×10⁻¹¹ compared to cells without rapamycin treatment ^{#}P=0.02 compared to 24 hR. FIG. 10C is a graph depicting time course of 3T3-L1 cell survival upon starvation up to 24 hours. 3T3-L1 preadipocytes were either untransfected (UT) or transfected with plasmids containing eGFP-only negative control (eGFP), wild-type human CREBRF (WT), or the p.Arg457Gln variant (p.Arg457Gln) and starved (cultured in HBSS). Values are means±SEM of 2 transfection replicates for each construct, 6 wells for each transfection and 3 technical (cell counting) replicates (n=2×6×3=36). FIG. 10D is a graph depicting quantification of the rates of cell death between 0-6 hours of starvation. 3T3-L1 preadipocytes were either untransfected (UT) or transfected with plasmids containing eGFP-only negative control (eGFP), wild-type human CREBRF (WT), or the p.Arg457Gln variant (p.Arg457Gln) and then cultured in HBSS. Values are means±SEM of 2 transfection replicates for each construct, 6 wells for each transfection and 3 technical (cell counting) replicates (n=2×6×3=36). This experiment was performed once following a pilot experiment with fewer time points showing similar results. Statistical analysis was performed using one way ANOVA and two-sided Games-Howell post hoc tests. ***P<5×10⁻⁵ compared to eGFP.

FIGS. 11A-11D depict evidence of positive selection centered on the missense variant rs373863828 (n=626 non-closely related Samoans). FIG. 11A depicts haplotype bifurcation plots for haplotypes carrying the ancestral allele. FIG. 11B depicts haplotype bifurcation plots for haplotypes carrying the derived allele. The haplotypes carrying the derived allele have unusual long-range homozygosity. FIG. 11C shows that the haplotypes carrying the derived allele have elevated extended haplotype homozygosity (EHH) values as one moves away from rs373863828 (vertical dotted line). FIG. 11D depicts that the haplotypes carrying the derived allele are longer than those carrying the ancestral allele.

FIGS. 12A and 12 B depict iHS and nSL scores in an 800 kb region centered on the missense variant rs373863828 (n=626 non-closely related Samoans). FIG. 12A is a graph depicting iHS scores versus physical position. FIG. 12B is a graph depicting nSL scores versus physical position. In both FIG. 12A and FIG. 12B, the blue dot indicates the score at the missense variant rs373863828, while the yellow dot indicates the score at the discovery variant rs12513649; the dotted horizontal line indicates the score at the missense variant rs373863828.

FIGS. 13A-13F are graphs depicting that Crebrf knockdown produces opposite effects to wide type Crebrf overexpression. 3T3-L1 adipocytes were transfected with an inducible shRNA construct targeting the Crebrf mRNA and shRNA expression was induced by the administration of 1.0 μg/ml doxycycline. Adipogenic gene expression (FIGS. 13A-13C), lipid accumulation (FIG. 13D), maximal respiration (FIG. 13E) and cell death rate upon starvation (FIG. 13F) was determined as described herein. Crebrf knockdown (KD) data were normalized to non-target control and compared to data from cells overexpressing wild-type (WT) or p.Arg457Gln variant (VAR) CREBRF normalized to their controls. The overexpression data is the same as described herein.

FIG. 14 is immunoblots depicting co-immunoprecipitation that showed that CREBRF binds another transcription factor, CREBL2, and this binding is enhanced by the or p.Arg457Gln variant. Cell extracts from 3T3-L1 cells overexpressing of human CREBRF with c-terminal Myc-His tag (Minster et al. 2016) were prepared using the IP Lysis solution included in the Pierce Co-IP kit (Thermo, Ill., USA). AminoLink Plus Coupling Resin suspension aliquots were coupled with 75 μg anti-Creb12 antibody (Sigma SAB1300866). The antibody coupled resin was incubated with cell lysates overnight at 4° C. with gentle rotation. Washing and elution were performed based on the manufacturer's instructions. Eluted proteins were mixed with sample buffer, separated by SDS-PAGE and probed using Myc antibody (Sigma M4439). Non-immune mouse IgG was used as control and aliquots of the cell extract were analyzed by immunoblotting as input controls.

FIGS. 15A-15E depict that CREBRF binding of target gene promoters upon starvation is enhanced by the variant. By chromatin immunoprecipitation, several target genes were identified that CREBRF can bind to. Binding of CREBRF to these genes was enhanced by starvation, and further enhanced by the p.Arg457Gln variant (denoted as “mutation” in the x axis labels). The target genes includes mSdhaf4 (FIG. 15A), mMme (FIG. 15B), mCrebl2 (FIG. 15C), mTbcel (FIG. 15D), and mCreg2 (FIG. 15E). Abbreviation: WT, wild-type.

FIG. 16A depicts the expression of CREBRF in human liver. Human tissues were obtained from the Origene Human tissue panel, n=1 per tissue. FIG. 16B depicts the expression of CREBRF in murine liver. Mouse tissues were obtained from 10 week-old, ad lib-fed, male C56BL/6J mice (n=6-12/group). Expression was normalized to the endogenous control gene peptidylprolyl isomerase A/cyclophilin A (PPIA for human; Ppia for mouse).

FIG. 17 is a graph depicting that the expression of CREBRF is nutritionally-regulated in murine liver. 24 week-old, male, C56BL/6J mice were fasted for 16 hours (n=6-7/group). Liver was collected and endogenous CREBRF mRNA was measured.

FIG. 18 is a graph depicting that the expression of CREBRF is induced by serum starvation and rapamycin (mTOR inhibition) and suppressed by insulin treatment in human HepG2 hepatocytes. Human HepG2 cells (17,000 cells/cm2) were treated with DMEM alone (4.5 g/L glucose, without FBS=Serum starvation), complete DMEM (4.5 g/L glucose with FBS) with 20 ng/ml Rapamycin or complete DMEM with 1 ug/ml Insulin for 0, 2, 6, 12, and 24h. A set of cells were also treated under the former conditions and then refed with complete DMEM for additional 12 h (24 hR). Cells were then collected for RNA preparation, cDNA synthesis, and gene expression analysis.

FIGS. 19A-19E are graphs and image depicting that overexpression of wild-type (WT) or variant (p.Arg457Gln) CREBRF influences hepatocellular lipid content, mitochondrial respiration, and cell survival. FIG. 19A depicts overexpression of wild-type (solid green) or R457Q variant (hatched green) CREBRF increased expression of CREBRF wild-type or variant mRNA 6-fold compared to endogenous CREBRF expression in human HepG2 hepatocytes. FIG. 19B is an image confirming expression wild-type (red) or R457Q variant (green) in human HepG2 hepatocytes. FIG. 19C shows that expression of wild-type and R457Q variant CREBRF increased triglyceride (TG) content in HepG2 hepatocytes. FIG. 19D depicts that expression of wild-type and R457Q variant CREBRF differentially influence mitochondrial respiration in HepG2 hepatocytes. FIG. 19E depicts that expression of wild-type CREBRF tends to improve and R457Q variant CREBRF significantly improves survival in HepG2 hepatocytes. As used herein, R457Q represents the variant p. Arg457Gln substitution and these two term are used interchangeably.

FIG. 20 depicts the primers and probes used in CRISPR/Cas9 mutagenesis and detection of the genetic manipulation in cells or cell/tissues from mice.

FIG. 21 depicts the nucleotide sequence of pReceiver-M10-CREBRF,transcript_variant1_WT., which is an expression vector encoding wild-type CREBRF.

FIG. 22 depicts the nucleotide sequence of Receiver-M10-CREBRF,transcript_variant1_p.R457Q, which is an expression vector encoding the CREBRF variant Arg457Gln.

### DETAILED DESCRIPTION OF THE INVENTION

The invention features compositions and methods that are useful for identifying a subject as having a genetic predisposition to obesity or at risk of developing obesity (e.g., BMI>30 kg/m²). The invention also provides compositions and methods for expressing a CREBRF polypeptide of the invention in an adipocyte or precursor thereof and cells expressing a nucleic acid molecule encoding a CREBRF polypeptide of the invention. The invention still further provides methods for identifying compounds that modulate adipogenesis (i.e., screening assays), as well as kits for practicing the methods of the inventions.

### Definitions

Before further description of the invention, certain terms employed in the specification, examples and appended claims are, for convenience, collected here.

By “adipocyte” is meant a cell that stores fat (e.g., triglycerides and cholesteryl ester). Adipocytes are the main constituent of body fat or adipose tissue.

By “adipogenesis” is meant the process in which a preadipocyte differentiates into an adipocyte.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “cell survival” is meant cell viability.

By “reducing cell death” is meant reducing the propensity or probability that a cell will die. Cell death can be apoptotic, necrotic, or by any other means.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CREB3 regulatory factor (CREBRF) polypeptide” is meant a polypeptide or fragment thereof having at least about 85% or greater amino acid identity to the amino acid sequence provided at NCBI Accession No. NP_705835 and having DNA binding, protein binding, and transcriptional regulatory activity. CREBRF binds CREB3, promotes CREB3 degradation, and represses CREB3 transcriptional activity. An exemplary CREBRF amino acid sequence having an arginine at position 457 is provided below:

| | |
|---|---|
| 1 | mpqpsvsgmd ppfgdafrsh tfseqtlmst dllanssdpd fmyeldremn yqqnprdnfl |
| 61 | sledckdien lesftdvldn egaltsnweq wdtycedltk ytkltscdiw gtkevdylgl |
| 121 | ddfsspyqde evisktptla qlnsedsqsv sdslyypdsl fsvkqnplps sfpgkkitsr |
| 181 | aaapvcsskt lqaevplsdc vqkaskptss tqimvktnmy hnekvnfhve ckdyvkkakv |
| 241 | kinpvqqsrp llsqihtdaa kentcycgav akrqekkgme plqghatpal pfketqelll |
| 301 | splpqegpgs laagesssls astsvsdssq kkeehnyslf vsdnlgeqpt kcspeedeed |
| 361 | eedvddedhd egfgsehels eneeeeeeee dyeddkdddi sdtfsepgye ndsvedlkev |
| 421 | tsissrkrgk rryfweyseq ltpsqqerml rpsewnrdtl psnmyqkngl hhgkyavkks |
| 481 | rrtdvedltp npkkllqign elrklnkvis dltpvselpl tarprsrkek nklasracrl |
| 541 | kkkaqyeank vklwglntey dnllfvinsi kqeivnrvqn prdergpnmg qkleilikdt |
| 601 | lglpvagqts efvnqvlekt aegnptgglv glriptskv |

An exemplary CREBRF amino acid sequence having a glutamine at position 457 is provided below:

| | |
|---|---|
| 1 | mpqpsvsgmd ppfgdafrsh tfseqtlmst dllanssdpd fmyeldremn yqqnprdnfl |
| 61 | sledckdien lesftdvldn egaltsnweq wdtycedltk ytkltscdiw gtkevdylgl |
| 121 | ddfsspyqde evisktptla qlnsedsqsv sdslyypdsl fsvkqnplps sfpgkkitsr |
| 181 | aaapvcsskt lqaevplsdc vqkaskptss tqimvktnmy hnekvnfhve ckdyvkkakv |
| 241 | kinpvqqsrp llsqihtdaa kentcycgav akrqekkgme plqghatpal pfketqelll |
| 301 | splpqegpgs laagesssls astsvsdssq kkeehnyslf vsdnlgeqpt kcspeedeed |
| 361 | eedvddedhd egfgsehels eneeeeeeee dyeddkdddi sdtfsepgye ndsvedlkev |
| 421 | tsissrkrgk rryfweyseq ltpsqqerml rpsewnqdtl psnmyqkngl hhgkyavkks |
| 481 | rrtdvedltp npkkllqign elrklnkvis dltpvselpl tarprsrkek nklasracrl |
| 541 | kkkaqyeank vklwglntey dnllfvinsi kqeivnrvqn prdergpnmg qkleilikdt |
| 601 | lglpvagqts efvnqvlekt aegnptgglv glriptskv |

By “CREBRF nucleic acid molecule” is meant a polynucleotide encoding a CREBRF polypeptide. An exemplary CREBRF nucleic acid molecule sequence is provided at NCBI Accession No. NM_153607. An exemplary CREBRF nucleic acid sequence having a G at nucleotide position 1689 is provided below:

| | |
|---|---|
| 1 | gagtcacgcg atttccggga acccgtcagg aaggacataa acaaaacaaa cccgaggcag |
| 61 | catggagagg ggccgtggcc cctgcagcgg aaccggaccc agtccctgag ccgcccctac |
| 121 | acccacagac agcatcgcac agaattattt taaaaaaaag cagtgatcca agcaattgaa |
| 181 | ttggaagcac tctggggaaa cctgctgttt attgtggaaa tcatcttcga tcttggaatt |
| 241 | gaaagtaaag ctggaaagga atttacaaac aagaaaaaaa agaagtttgg aatcggattc |
| 301 | acaggatctg ggcttggaaa tgcctcagcc tagtgtaagc ggaatggatc cgcctttcgg |
| 361 | ggatgccttt cgaagccaca ccttttcgga acaaactctg atgagcacag atctcttagc |
| 421 | aaacagttcg gatccagatt tcatgtatga actggataga gagatgaact accaacagaa |
| 481 | tcctagagac aactttcttt ctttggagga ctgcaaagac attgaaaatc tggagtcttt |
| 541 | cacagatgtc ctggataatg agggtgcttt aacctcaaac tgggaacagt gggatacata |
| 601 | ctgtgaagac ctaacgaaat ataccaaact aaccagctgt gacatctggg gaacaaaaga |
| 661 | agtggattac ttgggtcttg atgacttttc tagtccttac caagatgaag aggttataag |
| 721 | taaaactcca actttagctc aacttaatag tgaggactca cagtctgttt ctgattccct |
| 781 | ttattacccc gattcacttt tcagtgtcaa acaaaatccc ttaccctctt cattccctgg |
| 841 | taaaaagatc acaagcagag cagctgctcc tgtgtgttct tctaagactc tgcaggctga |
| 901 | ggtccctttg tcagactgtg tccaaaaagc aagtaaaccc acttcaagca cacaaatcat |
| 961 | ggtgaagacc aacatgtatc ataatgaaaa ggtgaacttt catgttgaat gtaaagacta |
| 1021 | tgtaaaaaag gcaaaggtaa agatcaaccc agtgcaacag agccggccct tgttgagcca |
| 1081 | gattcacaca gatgcagcaa aggagaacac ctgctactgt ggtgcagtgg caaagagaca |
| 1141 | agagaaaaaa gggatggagc ctcttcaagg tcatgccact cccgctttgc cttttaaaga |
| 1201 | aacccaggaa ctattactaa gtcccctgcc ccaggaaggt cctgggtcac ttgcagcagg |
| 1261 | agagagcagc agtctttctg ccagtacatc agtctcagat tcatcccaga aaaaagaaga |
| 1321 | gcacaattat tctctttttg tctccgacaa cttgggtgaa cagccaacta aatgcagtcc |
| 1381 | tgaagaagat gaggaggacg aggaggatgt tgatgatgag gaccatgatg aaggattcgg |
| 1441 | cagtgagcat gaactgtctg aaaatgagga ggaggaagaa gaggaagagg attatgaaga |
| 1501 | tgacaaggat gatgatatta gtgatacttt ctctgaacca ggctatgaaa atgattctgt |
| 1561 | agaagacctg aaggaggtga cttcaatatc ttcacggaag agaggtaaaa gaagatactt |
| 1621 | ctgggagtat agtgaacaac ttacaccatc acagcaagag aggatgctga gaccatctga |
| 1681 | gtggaaccga gatactttgc caagtaatat gtatcagaaa aatggcttac atcatggaaa |
| 1741 | atatgcagta aagaagtcac ggagaactga tgtagaagac ctgactccaa atcctaaaaa |
| 1801 | actcctccag ataggcaatg aacttcggaa actgaataag gtgattagtg acctgactcc |
| 1861 | agtcagtgag cttcccttaa cagcccgacc aaggtcaagg aaggaaaaaa ataagctggc |
| 1921 | ttccagagct tgtcggttaa agaagaaagc ccagtatgaa gctaataaag tgaaattatg |
| 1981 | gggcctcaac acagaatatg ataatttatt gtttgtaatc aactccatca agcaagagat |
| 2041 | tgtaaaccgg gtacagaatc caagagatga gagaggaccc aacatggggc agaagcttga |
| 2101 | aatcctcatt aaagatactc tcggtctacc agttgctggg caaacctcag aatttgttaa |
| 2161 | ccaagtgtta gagaagactg cagaagggaa tcccactgga ggccttgtag gattaaggat |
| 2221 | accaacatca aaggtgtaat cagcctcatt ggaccactgg tcagaaatgt ctgcgttttg |
| 2281 | tcacgttatc cattgtaaat tttcattctg ttttgcatgt cagttagcat tatgtaaaca |
| 2341 | tttacaatta ggttacattg ttttaagaac taagtagcat aagtgaagca tgatccaaaa |
| 2401 | tacttgatta ttgcattttc agagcataaa ccatgattaa aactgctact ggcatcagaa |
| 2461 | ttgaaaatca tatgtttaag taaatgttag gtacagatta caaaaatctg ttaaagcaaa |
| 2521 | acattttgga ggagtgaaat agtaaaatgc caagtattgt ggcagattta tgctctgaac |
| 2581 | cacacaaaaa aattgaggaa gcattttttt aaacagtcgg tttaaattgt ttttagaatt |
| 2641 | attgcttttt gttctaattt tccacaacca ttaatctcac ttgtatatgg cacacccagc |
| 2701 | acttgtgcct gtgggccata ttagatgttc attgtcagag ctcaagatga tatatataaa |
| 2761 | tatatatata tatatatata tatacacaca cacacacaaa tgtctgtgca agtaagaaaa |
| 2821 | aaaaagcata ttctttgtgc cttgtatttt ggggaaactc taaaactggt aatattttgt |
| 2881 | atgatgaaaa ccctaatgag aaaaaacaag atatatagat ggaaaaatta tggggtttaa |
| 2941 | atgttttttt gttccaactc tttttcagat tttttgaatg tatataggac tatgttgaaa |
| 3001 | tgtagatata tgccacagag tctgtgtatt gtataaaaaa caaaacaaaa aacaacaaaa |
| 3061 | aaaagatggc tctagaaaac tcatatttcg gtacttgacc ggaagaagac aaatacttgc |
| 3121 | acattattgc gattgtttta ttttttgtac caaagacaaa tgcaactgat atggcaaact |
| 3181 | gccagtctaa gtaaagtttt gcacagctta catgatactg tatgaatgta tgaaaaaaaa |
| 3241 | ggagaaaaaa aagaaaaaaa aaggtcaggg ttagggatct tactgaactg tgaattttat |
| 3301 | ttctgtttgg gtccaattat ctacagaagg agcatccata catacaaata ttattttgct |
| 3361 | gttcctctag ttcgcttcca tagtagataa gttggtggcc atttagatgt cttttatttc |
| 3421 | tgcacttatt gtaggaaatt ttaatatatt tcattttagt aagctattga taaaatagtt |
| 3481 | tttgactttg aaaattaaaa tgtttattta gcttattgta gtatacttcc accagacaac |
| 3541 | aaaatagatt atttttattg tattatgtat atatatatat gtaaagaaag aaaaaagcta |
| 3601 | aaaatatcta attctttagt tgccactttt ccgattgatg tattattgtg catgtaatat |
| 3661 | tttcaaagat caacacaggc taaaacaaaa acaatttata gatttttata tttttgtaca |
| 3721 | ggtattttca aactagcttc ttcaaactta acatgtgact tattcttcta tagtttctag |
| 3781 | aattgagaaa cattaacaca tttagttttt aggtgctctt ttttgctcat ataaaacagc |
| 3841 | ttcattagtc agtgttttaa ctgtgttcaa gctttacctc ttgatgagaa atttcttatg |
| 3901 | tcaaggcagc attataaacc ttcccccaca gatttttcca tcctgtctct cttactgttt |
| 3961 | tattctcaaa tcttgtgctt tgaactctga aaactggtgg cttaaaaact aaaaaaagaa |
| 4021 | aaaaagcata tttagcaagg aaaaaaatac caaaaatttc aggcatagct gctggaaaaa |
| 4081 | ttatctattt ctccattacc cactgtagga tttctttttt aattatactt tgactataaa |
| 4141 | gtgtcaaagt ataatttgtt cttttctttt actttgttac cccatttgta agctatagca |
| 4201 | tatgaagcta tatatatagc ttgtgaaggt ttgatctaga acacccagta acaaatgaac |
| 4261 | aatgttgctt acctgcttct ttgacatctt aaaaaagaaa tccaaggagg attgtaagga |
| 4321 | ttgtcttacc accttagctg aactgtgatg cacaagattt ttctatgtgt ttggtggaaa |
| 4381 | tgtacctggt ttgtacattc acgctaaaca gatgataagc tcaagtctga tggtttaata |
| 4441 | gaatgtaagt tcatcgttta aagcttttcc tttttaggtt ggagaaggca aaacacaggc |
| 4501 | ttgcaagttg gaagtatatg aagtcttgac agagtgtgtc tggtaaattg aaaagtgttt |
| 4561 | caaactatgg cagttttgca atcaggtgaa aatcacctca tgatattcag ctgataaggt |
| 4621 | ttataaaatt gcccctttct agctgctctg ttaggaattc tggtttttga tacttttttc |
| 4681 | ctgtctgcaa accagaattt gattttttgg tcttgcattt caaaaaaaaa aagactttga |
| 4741 | atctgtttag tagattccat atctttgagt ttcagtgttt tatatgtact acttaagtta |
| 4801 | aatagttaaa agcttttaaa tagttgagct ttttaatgtt gacactttat tttgtaccta |
| 4861 | tttatatatg tatgtatatc ttagaaaagc actttgttaa aaaaaaattg cattttatat |
| 4921 | gattcctgcc atttgctgct aaatctgggc tggtcagaat gctgcagcga tacttgatct |
| 4981 | atataaaaac ctggcagtaa aatgtagagt gaaagttaaa tcctcttgct gttttaactt |
| 5041 | tatcataaag atgacatagg caagctgtgc agctttacat tttaaccagg ggactctgtg |
| 5101 | gcatttaaaa ccgtctagaa atggttgtac tttaatgcca gtaataatct gcttcctcta |
| 5161 | ttgtcattaa aatatatacg tttagtgtat cacacaaacc aatcttataa gggtaatgta |
| 5221 | aaaaccccaa caattgtaca tgttctgttt ttgaaaattg tggcatgtat ttttgggtga |
| 5281 | agatcattag agaagagttc tctaaaggtt ttctgtgttc atacatggta tacagatagc |
| 5341 | tcataatgaa gtccagaatc ttacttttaa gtgaaggcat tgtgaattca cctcaagtaa |
| 5401 | acccattgtt ccaaagcaat tataaacttt gactctagta ctactatgat ttaaaaaaaa |
| 5461 | aaaaaaccaa caaaaacctt ttttcctagt ttcagataca ctggattctt tatagagttt |
| 5521 | gtctccatat gaaagcatgc tgtccagtcg ctcttgttaa gatcttgtct gagttttgaa |
| 5581 | ttgggtgcca cacttttcca gtcaatataa ttgcttgttc tactgtacca tgtatgattc |
| 5641 | ttgtcctttc ctatatcctt catgacagat tatgatgtgg ctttatattg tgccttactt |
| 5701 | gtacatttaa aactaaacgt cttcattccc ttccacttcc tacatcttta actttgacct |
| 5761 | ttttggtaag agaatcagaa ctattacaaa agcatcatga aggatttcag atgggtatgg |
| 5821 | tttcaaattc cctctcttta tagttatttt atatttgtat gaaagaccag ttttggatgg |
| 5881 | tctttgaata taggggggaa agattagcag taatttcact acatcccttt tctctgactt |
| 5941 | tcatgcattt ctcatacatc ttctttctga tgcttgactt tatttgcttc ctagcaatag |
| 6001 | tctgcattta aagaaaggtg tgttcaattc atcagcttga aattgactat ttcatttttc |
| 6061 | caggattttt taggagaaga gtacccattt tgttttataa aaacagatga caagtctctt |
| 6121 | taaaagaaac agaagtacag tacttttgaa atacaatgct gttagtttgg atttcttttt |
| 6181 | atatatatat ataatattca tacaatgatc tgatgtttgc cttcattaat aaagctgtta |
| 6241 | gtttattcac caaaatgtca agaatggatg tgcttttctt tattccacac atttaaaaaa |
| 6301 | atttagctgc taagatttaa tgttataaga aatgaattca agttgccttc agcaagaatt |
| 6361 | aacaaaaact tatgttccct ttctttatat agtttcctaa aattctgttc aagtattttc |
| 6421 | tagttaatta tgtaacagaa tgttagcatc tctccatatc ttgaaacttg aattttgaga |
| 6481 | atgcattgaa ttatgctttc agtgttaaag taaaaggttt caattatcct tctagtgaag |
| 6541 | tctgttgtgg aataccattt cccatggaac tgaggccatt tccacaactt tgcacagaac |
| 6601 | tgcagtcttg ttcttccctt ggatcatgac aaataagtct cacacagtgc cgtaatactt |
| 6661 | gtggattctt ttgtaatctt tgtaatctta ataagggcat tatgagaaga cgactccatg |
| 6721 | tttttttaat acttcaaaca cattgggatg taacaatgaa tgtcaactgt aggaatggtg |
| 6781 | gtttcgtttt aaggaataag catgttgggg aaagatgatg aaaatgtact actgaaagtt |
| 6841 | atacacttcc ataggcaaat gggattatgt gttgaagcat agtcctcatg cttaataaac |
| 6901 | tgactgaaat cgtagaaatt acacctagga actgagctag gccaaattgc catttttgtt |
| 6961 | tagagagttt tggaggtagt agtgagggga cagagcctta aaactacttc caaacagtat |
| 7021 | tttggaattg aagacttggt aactagtgaa gaacatcaaa gttgggtatt tcaatgtgcc |
| 7081 | aagtttgggt gaactaggtt cggtttgcct ctttcataac aatgtaaaca caatggtgta |
| 7141 | gttaattaaa ttctgggtgg ataggagcag gactgattac tatgtcttgc ccttcgccct |
| 7201 | ttgttttttt cagaaccaaa taacagaaat gtgtatgtgt gtactgtatc tgcctttcca |
| 7261 | ccacattttt atgacactgt attccactgc ctgctttttt accttctttc cctaggattt |
| 7321 | gtcctacagc ttagtattgt ggttgacagc gatactaggg ctgacagcac agaagtcaca |
| 7381 | agagaagagt ggaagggcaa gaattcaaag catttgttca tacaatgtgg caacctcttt |
| 7441 | tgcatagttg cgtaggatcc tgtttgtaat gctatcataa atattctgta gttttttttt |
| 7501 | tttctctccc aactggagct atgacacttt ttattggatt cagtcttgtc tcttgtctag |
| 7561 | aaagaacttt atcttgttga cgcatgagct gtttaaaaat tatcctatta aatgttggtt |
| 7621 | aatagttgtg cagtttttca tttcagatgg aaaggcaatg caaattttgc ctttgttttc |
| 7681 | tgtcaccttc caacccctga gcacttctag tcagatacag attcatcagt gtatgcaaca |
| 7741 | tcctttgtaa tttaaaataa aaaaagatga aaagaaaacg tt |

An exemplary CREBRF nucleic acid sequence having an A at nucleotide position 1689 is provided below:

| | |
|---|---|
| 1 | gagtcacgcg atttccggga acccgtcagg aaggacataa acaaaacaaa cccgaggcag |
| 61 | catggagagg ggccgtggcc cctgcagcgg aaccggaccc agtccctgag ccgcccctac |
| 121 | acccacagac agcatcgcac agaattattt taaaaaaaag cagtgatcca agcaattgaa |
| 181 | ttggaagcac tctggggaaa cctgctgttt attgtggaaa tcatcttcga tcttggaatt |
| 241 | gaaagtaaag ctggaaagga atttacaaac aagaaaaaaa agaagtttgg aatcggattc |
| 301 | acaggatctg ggcttggaaa tgcctcagcc tagtgtaagc ggaatggatc cgcctttcgg |
| 361 | ggatgccttt cgaagccaca ccttttcgga acaaactctg atgagcacag atctcttagc |
| 421 | aaacagttcg gatccagatt tcatgtatga actggataga gagatgaact accaacagaa |
| 481 | tcctagagac aactttcttt ctttggagga ctgcaaagac attgaaaatc tggagtcttt |
| 541 | cacagatgtc ctggataatg agggtgcttt aacctcaaac tgggaacagt gggatacata |
| 601 | ctgtgaagac ctaacgaaat ataccaaact aaccagctgt gacatctggg gaacaaaaga |
| 661 | agtggattac ttgggtcttg atgacttttc tagtccttac caagatgaag aggttataag |
| 721 | taaaactcca actttagctc aacttaatag tgaggactca cagtctgttt ctgattccct |
| 781 | ttattacccc gattcacttt tcagtgtcaa acaaaatccc ttaccctctt cattccctgg |
| 841 | taaaaagatc acaagcagag cagctgctcc tgtgtgttct tctaagactc tgcaggctga |
| 901 | ggtccctttg tcagactgtg tccaaaaagc aagtaaaccc acttcaagca cacaaatcat |
| 961 | ggtgaagacc aacatgtatc ataatgaaaa ggtgaacttt catgttgaat gtaaagacta |
| 1021 | tgtaaaaaag gcaaaggtaa agatcaaccc agtgcaacag agccggccct tgttgagcca |
| 1081 | gattcacaca gatgcagcaa aggagaacac ctgctactgt ggtgcagtgg caaagagaca |
| 1141 | agagaaaaaa gggatggagc ctcttcaagg tcatgccact cccgctttgc cttttaaaga |
| 1201 | aacccaggaa ctattactaa gtcccctgcc ccaggaaggt cctgggtcac ttgcagcagg |
| 1261 | agagagcagc agtctttctg ccagtacatc agtctcagat tcatcccaga aaaaagaaga |
| 1321 | gcacaattat tctctttttg tctccgacaa cttgggtgaa cagccaacta aatgcagtcc |
| 1381 | tgaagaagat gaggaggacg aggaggatgt tgatgatgag gaccatgatg aaggattcgg |
| 1441 | cagtgagcat gaactgtctg aaaatgagga ggaggaagaa gaggaagagg attatgaaga |
| 1501 | tgacaaggat gatgatatta gtgatacttt ctctgaacca ggctatgaaa atgattctgt |
| 1561 | agaagacctg aaggaggtga cttcaatatc ttcacggaag agaggtaaaa gaagatactt |
| 1621 | ctgggagtat agtgaacaac ttacaccatc acagcaagag aggatgctga gaccatctga |
| 1681 | gtggaaccaa gatactttgc caagtaatat gtatcagaaa aatggcttac atcatggaaa |
| 1741 | atatgcagta aagaagtcac ggagaactga tgtagaagac ctgactccaa atcctaaaaa |
| 1801 | actcctccag ataggcaatg aacttcggaa actgaataag gtgattagtg acctgactcc |
| 1861 | agtcagtgag cttcccttaa cagcccgacc aaggtcaagg aaggaaaaaa ataagctggc |
| 1921 | ttccagagct tgtcggttaa agaagaaagc ccagtatgaa gctaataaag tgaaattatg |
| 1981 | gggcctcaac acagaatatg ataatttatt gtttgtaatc aactccatca agcaagagat |
| 2041 | tgtaaaccgg gtacagaatc caagagatga gagaggaccc aacatggggc agaagcttga |
| 2101 | aatcctcatt aaagatactc tcggtctacc agttgctggg caaacctcag aatttgttaa |
| 2161 | ccaagtgtta gagaagactg cagaagggaa tcccactgga ggccttgtag gattaaggat |
| 2221 | accaacatca aaggtgtaat cagcctcatt ggaccactgg tcagaaatgt ctgcgttttg |
| 2281 | tcacgttatc cattgtaaat tttcattctg ttttgcatgt cagttagcat tatgtaaaca |
| 2341 | tttacaatta ggttacattg ttttaagaac taagtagcat aagtgaagca tgatccaaaa |
| 2401 | tacttgatta ttgcattttc agagcataaa ccatgattaa aactgctact ggcatcagaa |
| 2461 | ttgaaaatca tatgtttaag taaatgttag gtacagatta caaaaatctg ttaaagcaaa |
| 2521 | acattttgga ggagtgaaat agtaaaatgc caagtattgt ggcagattta tgctctgaac |
| 2581 | cacacaaaaa aattgaggaa gcattttttt aaacagtcgg tttaaattgt ttttagaatt |
| 2641 | attgcttttt gttctaattt tccacaacca ttaatctcac ttgtatatgg cacacccagc |
| 2701 | acttgtgcct gtgggccata ttagatgttc attgtcagag ctcaagatga tatatataaa |
| 2761 | tatatatata tatatatata tatacacaca cacacacaaa tgtctgtgca agtaagaaaa |
| 2821 | aaaaagcata ttctttgtgc cttgtatttt ggggaaactc taaaactggt aatattttgt |
| 2881 | atgatgaaaa ccctaatgag aaaaaacaag atatatagat ggaaaaatta tggggtttaa |
| 2941 | atgttttttt gttccaactc tttttcagat tttttgaatg tatataggac tatgttgaaa |
| 3001 | tgtagatata tgccacagag tctgtgtatt gtataaaaaa caaaacaaaa aacaacaaaa |
| 3061 | aaaagatggc tctagaaaac tcatatttcg gtacttgacc ggaagaagac aaatacttgc |
| 3121 | acattattgc gattgtttta ttttttgtac caaagacaaa tgcaactgat atggcaaact |
| 3181 | gccagtctaa gtaaagtttt gcacagctta catgatactg tatgaatgta tgaaaaaaaa |
| 3241 | ggagaaaaaa aagaaaaaaa aaggtcaggg ttagggatct tactgaactg tgaattttat |
| 3301 | ttctgtttgg gtccaattat ctacagaagg agcatccata catacaaata ttattttgct |
| 3361 | gttcctctag ttcgcttcca tagtagataa gttggtggcc atttagatgt cttttatttc |
| 3421 | tgcacttatt gtaggaaatt ttaatatatt tcattttagt aagctattga taaaatagtt |
| 3481 | tttgactttg aaaattaaaa tgtttattta gcttattgta gtatacttcc accagacaac |
| 3541 | aaaatagatt atttttattg tattatgtat atatatatat gtaaagaaag aaaaaagcta |
| 3601 | aaaatatcta attctttagt tgccactttt ccgattgatg tattattgtg catgtaatat |
| 3661 | tttcaaagat caacacaggc taaaacaaaa acaatttata gatttttata tttttgtaca |
| 3721 | ggtattttca aactagcttc ttcaaactta acatgtgact tattcttcta tagtttctag |
| 3781 | aattgagaaa cattaacaca tttagttttt aggtgctctt ttttgctcat ataaaacagc |
| 3841 | ttcattagtc agtgttttaa ctgtgttcaa gctttacctc ttgatgagaa atttcttatg |
| 3901 | tcaaggcagc attataaacc ttcccccaca gatttttcca tcctgtctct cttactgttt |
| 3961 | tattctcaaa tcttgtgctt tgaactctga aaactggtgg cttaaaaact aaaaaaagaa |
| 4021 | aaaaagcata tttagcaagg aaaaaaatac caaaaatttc aggcatagct gctggaaaaa |
| 4081 | ttatctattt ctccattacc cactgtagga tttctttttt aattatactt tgactataaa |
| 4141 | gtgtcaaagt ataatttgtt cttttctttt actttgttac cccatttgta agctatagca |
| 4201 | tatgaagcta tatatatagc ttgtgaaggt ttgatctaga acacccagta acaaatgaac |
| 4261 | aatgttgctt acctgcttct ttgacatctt aaaaaagaaa tccaaggagg attgtaagga |
| 4321 | ttgtcttacc accttagctg aactgtgatg cacaagattt ttctatgtgt ttggtggaaa |
| 4381 | tgtacctggt ttgtacattc acgctaaaca gatgataagc tcaagtctga tggtttaata |
| 4441 | gaatgtaagt tcatcgttta aagcttttcc tttttaggtt ggagaaggca aaacacaggc |
| 4501 | ttgcaagttg gaagtatatg aagtcttgac agagtgtgtc tggtaaattg aaaagtgttt |
| 4561 | caaactatgg cagttttgca atcaggtgaa aatcacctca tgatattcag ctgataaggt |
| 4621 | ttataaaatt gcccctttct agctgctctg ttaggaattc tggtttttga tacttttttc |
| 4681 | ctgtctgcaa accagaattt gattttttgg tcttgcattt caaaaaaaaa aagactttga |
| 4741 | atctgtttag tagattccat atctttgagt ttcagtgttt tatatgtact acttaagtta |
| 4801 | aatagttaaa agcttttaaa tagttgagct ttttaatgtt gacactttat tttgtaccta |
| 4861 | tttatatatg tatgtatatc ttagaaaagc actttgttaa aaaaaaattg cattttatat |
| 4921 | gattcctgcc atttgctgct aaatctgggc tggtcagaat gctgcagcga tacttgatct |
| 4981 | atataaaaac ctggcagtaa aatgtagagt gaaagttaaa tcctcttgct gttttaactt |
| 5041 | tatcataaag atgacatagg caagctgtgc agctttacat tttaaccagg ggactctgtg |
| 5101 | gcatttaaaa ccgtctagaa atggttgtac tttaatgcca gtaataatct gcttcctcta |
| 5161 | ttgtcattaa aatatatacg tttagtgtat cacacaaacc aatcttataa gggtaatgta |
| 5221 | aaaaccccaa caattgtaca tgttctgttt ttgaaaattg tggcatgtat ttttgggtga |
| 5281 | agatcattag agaagagttc tctaaaggtt ttctgtgttc atacatggta tacagatagc |
| 5341 | tcataatgaa gtccagaatc ttacttttaa gtgaaggcat tgtgaattca cctcaagtaa |
| 5401 | acccattgtt ccaaagcaat tataaacttt gactctagta ctactatgat ttaaaaaaaa |
| 5461 | aaaaaaccaa caaaaacctt ttttcctagt ttcagataca ctggattctt tatagagttt |
| 5521 | gtctccatat gaaagcatgc tgtccagtcg ctcttgttaa gatcttgtct gagttttgaa |
| 5581 | ttgggtgcca cacttttcca gtcaatataa ttgcttgttc tactgtacca tgtatgattc |
| 5641 | ttgtcctttc ctatatcctt catgacagat tatgatgtgg ctttatattg tgccttactt |
| 5701 | gtacatttaa aactaaacgt cttcattccc ttccacttcc tacatcttta actttgacct |
| 5761 | ttttggtaag agaatcagaa ctattacaaa agcatcatga aggatttcag atgggtatgg |
| 5821 | tttcaaattc cctctcttta tagttatttt atatttgtat gaaagaccag ttttggatgg |
| 5881 | tctttgaata taggggggaa agattagcag taatttcact acatcccttt tctctgactt |
| 5941 | tcatgcattt ctcatacatc ttctttctga tgcttgactt tatttgcttc ctagcaatag |
| 6001 | tctgcattta aagaaaggtg tgttcaattc atcagcttga aattgactat ttcatttttc |
| 6061 | caggattttt taggagaaga gtacccattt tgttttataa aaacagatga caagtctctt |
| 6121 | taaaagaaac agaagtacag tacttttgaa atacaatgct gttagtttgg atttcttttt |
| 6181 | atatatatat ataatattca tacaatgatc tgatgtttgc cttcattaat aaagctgtta |
| 6241 | gtttattcac caaaatgtca agaatggatg tgcttttctt tattccacac atttaaaaaa |
| 6301 | atttagctgc taagatttaa tgttataaga aatgaattca agttgccttc agcaagaatt |
| 6361 | aacaaaaact tatgttccct ttctttatat agtttcctaa aattctgttc aagtattttc |
| 6421 | tagttaatta tgtaacagaa tgttagcatc tctccatatc ttgaaacttg aattttgaga |
| 6481 | atgcattgaa ttatgctttc agtgttaaag taaaaggttt caattatcct tctagtgaag |
| 6541 | tctgttgtgg aataccattt cccatggaac tgaggccatt tccacaactt tgcacagaac |
| 6601 | tgcagtcttg ttcttccctt ggatcatgac aaataagtct cacacagtgc cgtaatactt |
| 6661 | gtggattctt ttgtaatctt tgtaatctta ataagggcat tatgagaaga cgactccatg |
| 6721 | tttttttaat acttcaaaca cattgggatg taacaatgaa tgtcaactgt aggaatggtg |
| 6781 | gtttcgtttt aaggaataag catgttgggg aaagatgatg aaaatgtact actgaaagtt |
| 6841 | atacacttcc ataggcaaat gggattatgt gttgaagcat agtcctcatg cttaataaac |
| 6901 | tgactgaaat cgtagaaatt acacctagga actgagctag gccaaattgc catttttgtt |
| 6961 | tagagagttt tggaggtagt agtgagggga cagagcctta aaactacttc caaacagtat |
| 7021 | tttggaattg aagacttggt aactagtgaa gaacatcaaa gttgggtatt tcaatgtgcc |
| 7081 | aagtttgggt gaactaggtt cggtttgcct ctttcataac aatgtaaaca caatggtgta |
| 7141 | gttaattaaa ttctgggtgg ataggagcag gactgattac tatgtcttgc ccttcgccct |
| 7201 | ttgttttttt cagaaccaaa taacagaaat gtgtatgtgt gtactgtatc tgcctttcca |
| 7261 | ccacattttt atgacactgt attccactgc ctgctttttt accttctttc cctaggattt |
| 7321 | gtcctacagc ttagtattgt ggttgacagc gatactaggg ctgacagcac agaagtcaca |
| 7381 | agagaagagt ggaagggcaa gaattcaaag catttgttca tacaatgtgg caacctcttt |
| 7441 | tgcatagttg cgtaggatcc tgtttgtaat gctatcataa atattctgta gttttttttt |
| 7501 | tttctctccc aactggagct atgacacttt ttattggatt cagtcttgtc tcttgtctag |
| 7561 | aaagaacttt atcttgttga cgcatgagct gtttaaaaat tatcctatta aatgttggtt |
| 7621 | aatagttgtg cagtttttca tttcagatgg aaaggcaatg caaattttgc ctttgttttc |
| 7681 | tgtcaccttc caacccctga gcacttctag tcagatacag attcatcagt gtatgcaaca |
| 7741 | tcctttgtaa tttaaaataa aaaaagatga aaagaaaacg tt |

By “rs373863828” is meant a single nucleotide polymorphism (SNP) 1689G→A in CREBRF, resulting in an arginine to glutamine change (R457Q) in the CREBRF polypeptide.

By “Cyclic AMP-responsive element-binding protein 3 (CREB3) polypeptide” is meant a polypeptide or fragment thereof having at least about 85% or greater amino acid identity to the amino acid sequence provided at NCBI Accession No. NP_006359 and having DNA binding, protein binding, and transcriptional regulatory activities. An exemplary CREB3 amino acid sequence is provided below:

| | |
|---|---|
| 1 | meleldagdq dllafllees gdlgtapdea vrapldwalp lsevpsdwev ddllcsllsp |
| 61 | paslnilsss npclvhhdht yslpretvsm dlesescrke gtqmtpqhme elaeqeiarl |
| 121 | vltdeeksll ekeglilpet lpltkteeqi lkrvrrkirn krsaqesrrk kkvyvggles |
| 181 | rvlkytaqnm elqnkvqlle eqnlslldql rklqamviei snktsssstc ilvllvsfcl |
| 241 | llvpamyssd trgslpaehg vlsrqlralp sedpyqlelp alqsevpkds thqwldgsdc |
| 301 | vlqapgntsc llhympqaps aepplewpfp dlfseplcrg pilplqanlt rkggwlptgs |
| 361 | psvilqdrys g |

By “CREB3 nucleic acid molecule” is meant a polynucleotide encoding a CREB3 polypeptide. An exemplary CREB3 nucleic acid molecule sequence is provided at NCBI Accession No. NM_{—} 006368. An exemplary CREB3 nucleic acid sequence is provided below:

| | |
|---|---|
| 1 | ggaagcgagg gtgcggcgca atccggagag gacgccagga cgacgcccga gttccctttc |
| 61 | aggctagaac tcttcctttt tctagcttgg ggtagaaggc ggagccggag ccccggaacc |
| 121 | cccgccctcg gggtgcgagg cggcagcagg gccgtcccct acatttgcat agcccctggg |
| 181 | acgtggcgct gcacccaagc ctcttctcag ttggagggaa ctccaagtcc cacagtgcca |
| 241 | cggggtgggg tgcgtcactt tcgctgcgtt ggaggctgag gagaattgag cctgggaggc |
| 301 | gggtccggag agggctatgg aaagccgccg gcggggaatc ccggccgtag agggacagtg |
| 361 | gataggtgcc cgaggcctac agctggcctg gggctcgtgt ctgggcttcg gacgttgggg |
| 421 | cccggtggcc caccctttcc gtagttgtcc caaatggagc tggaattgga tgctggtgac |
| 481 | caagacctgc tggccttcct gctagaggaa agtggagatt tggggacggc acccgatgag |
| 541 | gccgtgaggg ccccactgga ctgggcgctg ccgctttctg aggtaccgag cgactgggaa |
| 601 | gtagatgatt tgctgtgctc cctgctgagt cccccagcgt cgttgaacat tctcagctcc |
| 661 | tccaacccct gccttgtcca ccatgaccac acctactccc tcccacggga aactgtctct |
| 721 | atggatctag agagtgagag ctgtagaaaa gaggggaccc agatgactcc acagcatatg |
| 781 | gaggagctgg cagagcagga gattgctagg ctagtactga cagatgagga gaagagtcta |
| 841 | ttggagaagg aggggcttat tctgcctgag acacttcctc tcactaagac agaggaacaa |
| 901 | attctgaaac gtgtgcggag gaagattcga aataaaagat ctgctcaaga gagccgcagg |
| 961 | aaaaagaagg tgtatgttgg gggtttagag agcagggtct tgaaatacac agcccagaat |
| 1021 | atggagcttc agaacaaagt acagcttctg gaggaacaga atttgtccct tctagatcaa |
| 1081 | ctgaggaaac tccaggccat ggtgattgag atatcaaaca aaaccagcag cagcagcacc |
| 1141 | tgcatcttgg tcctactagt ctccttctgc ctcctccttg tacctgctat gtactcctct |
| 1201 | gacacaaggg ggagcctgcc agctgagcat ggagtgttgt cccgccagct tcgtgccctc |
| 1261 | cccagtgagg acccttacca gctggagctg cctgccctgc agtcagaagt gccgaaagac |
| 1321 | agcacacacc agtggttgga cggctcagac tgtgtactcc aggcccctgg caacacttcc |
| 1381 | tgcctgctgc attacatgcc tcaggctccc agtgcagagc ctcccctgga gtggccattc |
| 1441 | cctgacctct tctcagagcc tctctgccga ggtcccatcc tccccctgca ggcaaatctc |
| 1501 | acaaggaagg gaggatggct tcctactggt agcccctctg tcattttgca ggacagatac |
| 1561 | tcaggctaga tatgaggata tgtggggggt ctcagcagga gcctgggggg ctccccatct |
| 1621 | gtgtccaaat aaaaagcggt gggcaagggc tggccgcagc tcctgtgccc tgtcaggacg |
| 1681 | actgagggct caaacacacc acacttaatg gctttctggg tcttttattt gtacccatgt |
| 1741 | gtctgtcaca ccatgaatgt acctggggaa atcaactgac ctccctgaac atttcacgca |
| 1801 | gtcagggaac aggtgaggaa agaaataaat aagtgattct aatgctgcct aaaaaaaaaa |
| 1861 | aaaaaaaa |

“Derived from” as used herein refers to the process of obtaining a cell from a subject, embryo, biological sample, or cell culture.

“Detect” refers to identifying the presence, absence or amount of the object to be detected.

By “detectable reporter” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In certain embodiments, the fragment retains the activity of the polypeptide or nucleic acid molecule of which it is a fragment.

As used herein, “recombinant” includes reference to a polypeptide produced using cells that express a heterologous polynucleotide encoding the polypeptide. The cells produce the recombinant polypeptide because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, under-expressed or not expressed at all.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. It need not be purified to homogeneity. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By an “isolated cell” is meant a cell of the invention that has been separated from components that naturally accompany it, including, e.g., cells and cellular debris. In one embodiment, the disclosure provides an isolated cell comprising a nucleic acid sequence as disclosed herein.

By “marker” is meant any protein or polynucleotide having an alteration in expression level or activity that is associated with a disease or disorder. As used herein, “obtaining” as in “obtaining an agent” includes synthesizing, purchasing, or otherwise acquiring the agent.

The term “obesity” as used herein, refers to a condition characterized by the accumulation of excess body fat. Obesity can have a negative effect on health, leading to reduced life expectancy and/or increased health problems. Obesity may be evaluated by assessing a subject's body mass index (BMI), which is obtained by dividing a subject's weight by the square of the subject's height and/or by assessing fat distribution via the waist-hip ratio and total cardiovascular risk factor. A BMI between 18.50-24.99 kg/m² classifies an individual as having normal weight, between 25.00-29.99 kg/m² as being overweight, and exceeding 30 kg/m² as being obese.

By “promoter” is meant a promoter, e.g., a viral promoter, that is capable of initiating expression in a cell. Such cells include cells selected from the group consisting of a preadipocyte, an adipocyte, an hepatocyte (e.g., an HepG2 cell) and precursors thereof. In various embodiments, cell specific promoters are capable of initiating expression of that cell. In certain embodiments, such cells are mammalian cells (e.g., human cells).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition. As is apparent to one skilled in the art, an appropriate reference is where an element is changed in order to determine the effect of the element.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “subject” is meant a mammal, including, but not limited to, a human or non-human mammal, such as a bovine, porcine, equine, canine, ovine, murine or feline.

By “modulator” is meant any compound/agent that alters a biological function or activity of a cell. A modulator includes, without limitation, compounds/agents that reduce or eliminate a biological function or activity of a cell (e.g., an “inhibitor”). For example, a modulator may inhibit adipogenesis of a cell. A modulator includes, without limitation, compounds/agents that enhance or increase a biological function or activity of a cell. For example, a modulator may promote adipogenesis of a cell.

The term “modulate” is intended to encompass, in its various grammatical forms (e.g., “modulated”, “modulation”, “modulating”, etc.), up-regulation, induction, stimulation, potentiation, localization changes (e.g., movement of a protein from one cellular compartment to another) and/or relief of inhibition, as well as inhibition and/or down-regulation.

The term “compound” is intended include, but is not limited to, peptides, nucleic acids, carbohydrates, non-peptidic compounds, and natural product extracts.

The term “non-peptidic compound” is intended to encompass compounds that are comprised, at least in part, of molecular structures different from naturally-occurring L-amino acid residues linked by natural peptide bonds. However, “non-peptidic compounds” are intended to include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds, for example small organic molecules. “Non-peptidic compounds” also are intended to include natural products.

The terms “compound” and “agent” are used interchangeably in the context of the invention.

The terms “operably linked” is intended to mean that molecules are functionally coupled to each other in that the change of activity or state of one molecule is affected by the activity or state of the other molecule. For example, an adipocyte specific promoter operably linked to a nucleic acid sequence encoding a CREBRF polypeptide

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The invention provides polynucleotides and polypeptides described herein, and fragments thereof; and polynucleotides and polypeptides that are substantially identical the polynucleotides and polypeptides described herein.

By “target gene” is meant a gene, the expression of which is directly or indirectly regulated by CREBRF. For example, CREB3 is a target gene directly regulated by CREBRF. The expression of an adipogenic marker gene, such as Pparg2, Cebpa, or Adipoq, can be directly or indirectly regulated by CREBRF and these adipogenic marker genes are also target genes. In one embodiment, CREBRF regulates a gene by binding to the gene's promoter.

By “transgenic” is meant any cell which includes a DNA sequence which is inserted by artifice into a cell and becomes part of the genome of the organism which develops from that cell. As used herein, the transgenic organisms are generally transgenic mammalian (e.g., rodents such as rats or mice) and the DNA (transgene) is inserted by artifice into the nuclear genome. In one embodiment, the transgenic mouse is a knock-in mouse comprising an p.Arg457Gln mutation in CREBRF gene.

As used herein the term “knock-in” is intended to encompass a genetic engineering method that involves the one-for-one substitution of DNA sequence information with a wild-type copy in a genetic locus or the insertion of sequence information not found within the locus. Typically, this is done in mice because the technology for this process is more refined and there is a high degree of shared sequence complexity between mice and humans. The difference between knock-in technology and traditional transgenic techniques is that a knock-in involves a gene inserted into a specific locus, and is thus a “targeted” insertion. The knock-in mice disclosed herein provide disease models for obesity and allow for the study of the function of the regulatory machinery (e.g. promoters) that governs the expression of the natural gene being replaced. This is accomplished by observing the new phenotype of the organism in question.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “reduce” or “reduces” is meant a negative alteration of at least 10%, 25%, 50%, 75%, or 100%.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The invention is based, at least in part, on the discovery of a CREBRF variant resulting in an Arg457Gln mutation that was strongly associated with body mass index (BMI) (P=5.3×10⁻¹⁴), in a genome-wide association study (GWAS) of obesity-related traits conducted in 3,072 individuals from Samoa. This finding was replicated (P=1.2×10⁻⁹) in other samples from Samoa and American Samoa. Targeted sequencing analysis revealed that this signal is associated with the missense variant rs373863828 (p.Arg457Gln) in CREBRF (meta P=1.4×10⁻²⁰). This variant is common in Samoans (allele frequency of 0.259), but rare in people of African or European descent. In Samoans, each copy of the minor allele increases BMI by 1.58 kg/m² in females and 0.83 kg/m² in males, an effect size that is much larger than currently known common BMI risk variants. In the 3T3-L1 preadipocyte cell model, over-expression of both wild-type (WT) and p.Arg457Gln CREBRF human variants promoted adipogenesis in the absence of standard hormonal stimulation and enhanced cell survival in response to nutrition stress. However, compared to WT CREBRF, the p.Arg457Gln CREBRF variant had greater lipid accumulation and lower energy utilization, indicating that p.Arg457Gln is a “thrifty” variant that strongly influences obesity in humans.

### Nucleic Acids, Cloning and Expression Systems

The present disclosure further provides isolated nucleic acids encoding the disclosed CREBRF polypeptides and fragments thereof. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The present disclosure also provides constructs in the form of plasmids, vectors, phagemids, transcription or expression cassettes that comprise at least one nucleic acid encoding a CREBRF polypeptide or a fragment thereof, disclosed herein. The disclosure further provides a host cell that comprises one or more constructs as above.

Systems for cloning and expression of a polypeptide in a variety of different host cells are well known in the art. For cells suitable for producing polypeptides, see Gene Expression Systems, Academic Press, eds. Fernandez et al., 1999. Briefly, suitable host cells include, but are not limited to yeast, plant, algae, bacterial, mammalian, and insect cells. Mammalian cell lines available in the art for expression of a heterologous polypeptide include Chinese hamster ovary cells, HeLa cells, baby hamster kidney cells, NS0 mouse myeloma cells, and many others. A common bacterial host is E. coli. Any protein expression system compatible with the invention may be used to produce the disclosed proteins. Suitable expression systems include transgenic animals described in Gene Expression Systems, Academic Press, eds. Fernandez et al., 1999.

Suitable vectors can be chosen or constructed, so that they contain appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, for example, Sambrook et al., Molecular Cloning: A Laboratory Manual, 2nd ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd Edition, eds. Ausubel et al., John Wiley & Sons, 1992.

A still further aspect provides a method comprising introducing such a nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene. A wide variety of host cells are available for expressing CREBRF polypeptide mutants of the present invention. Such host cells include, for example, yeast, plant, algae, bacterial, mammalian, and insect cells.

The invention provides methods for enhancing adipogenesis in a cell (e.g., preadipocyte or other adipocyte precursor), comprising expressing or overexpressing a CREBRF polypeptide (e.g., a CREBRF polypeptide having a glutamine at amino acid position 457).

Transducing viral (e.g., retroviral, adenoviral, and adeno-associated viral) vectors can be used to express CREBRF in a cell, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a CREBRF polypeptide, variant, or fragment thereof, can be cloned into a retroviral vector and expression can be driven from its endogenous promoter, from the retroviral long terminal repeat, or from a promoter specific for a target cell type of interest, such as an adipocyte.

Other viral vectors that can be used in the methods of the invention include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In one embodiment, an adeno-associated viral vector (e.g., serotype 2) is used to administer a polynucleotide to an adipocyte or precursor thereof.

Non-viral approaches can also be employed for the introduction of a CREBRF polynucleotide into an adipocyte or precursor thereof. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In one embodiment, the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell (e.g., a preadipocyte, adipocyte, or precursor thereof).

### Genotyping of CREBRF Polymorphisms

The present invention provides a number of diagnostic assays that are useful for characterizing the genotype of a subject. Desirably, the methods of the invention discriminate between polymorphisms of a gene of interest. Preferably, both alleles corresponding to a gene of interest are identified. Accordingly, the invention provides for genotyping useful in virtually any clinical setting where conventional methods of analysis are used. In various aspects, the methods of the invention determine or detect CREBRF genetic variants at the SNP rs373863828. Results obtained from CREBRF genotyping at SNP rs373863828 may be used to select an appropriate therapy for a subject.

The presence or absence of SNP rs373863828 in the CREBRF gene may be evaluated using various techniques. In certain embodiments, PCR or real-time PCR may be used to detect a single nucleotide polymorphism. Polymerase chain reaction (PCR) is widely known in the art. See for example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2nd Ed., Springer-Verlag (New York: 1997). Various real-time PCR testing platforms that may be used with the present invention include: 5′ nuclease (TaqMan® probes), molecular beacons, and FRET hybridization probes. In certain embodiments, genotyping is performed using a TaqMan® assay, involving amplifying a CREBRF nucleic acid sequence, e.g., 5% AAGGCTATGAAAATGATTCTGTAGAAGACCTGAAGGAGGTGACTTC AATATCTTCACGGAAGAGAGGTAAAAGAAGATACTTCTGGGAGTATAGTGAACAACTT ACACCATCACAGCAAGAGAGGATGCTGAGACCATCTGAGTGGAACC[A/G]AGATACTTT GCCAAGTAATATGTATCAGAAAAATGGCTTACATCATGGTAAGAGGGGATTGCAGTCA GATATTTAGTGTCACTTTAATCAAGTTGAGCTACTAATCCATAATGTTTACTCCGTGTAC CTA-3′, where the SNP (rs373863828; denoted in brackets) in the sequence above is detected by using the following TaqMan primers and probe sequences:

Forward Primer:
5′-CAAGAGAGGATGCTGAGACCAT-3′
Reverse Primer:
5′-ACCATGATGTAAGCCATTTTTCTGATACA-3′
FAM ™(G) Probe:
5′-AGTGGAACCGAGATAC-3′
VIC ® (A) Probe:
5′-AGTGGAACCAAGATAC-3′

In other embodiments, a polymorphism may be detected using a technique including hybridization with a probe specific for SNP rs373863828, restriction endonuclease digestion, nucleic acid sequencing, primer extension, microarray or gene chip analysis, mass spectrometry, or a DNAse protection assay. In other embodiments, DNA sequencing may be used to evaluate a polymorphism of the present invention. Sequencing techniques, such as the Sanger method, are well known to those of skill in the art. Next-generation sequencing techniques may be used that do not fall within the scope of Sanger sequencing, including for example microarray sequencing, Solexa sequencing (Illumina), Ion Torrent (Life Technologies), SOliD (Applied Biosystems), pyrosequencing (based on the detection of released pyrophosphate (PPI); see U.S. Pat. Publ. No. 2006008824; herein incorporated by reference), Single-molecule real-time sequencing (Pacific Bio) or other sequencing techniques being developed, including for example, nanopore sequencing and tunnelling currents sequencing.

The genotyping methods of the invention involve detecting or determining a genetic variant or biomarker of interest in a biological sample. In one embodiment, the biologic sample contains a cell having diploid DNA content. Human cells containing 46 chromosomes (e.g., human somatic cells) are diploid. In one embodiment, the biologic sample is a tissue sample that includes diploid cells of a tissue (epithelial cells) or organ (e.g., skin cells). Such tissue is obtained, for example, from a cheek swab or biopsy of a tissue or organ. In another embodiment, the biologic sample is a biologic fluid sample. Biological fluid samples containing diploid cells include saliva, blood, blood serum, plasma, urine, hair follicle, or any other biological fluid useful in the methods of the invention.

### Inhibitory Nucleic Acids

As reported herein below, the disruption of CREBRF gene function results reduced adipogenesis. Accordingly, the invention provides oligonucleotides that inhibit the expression of CREBRF. Such inhibitory nucleic acid molecules include single and double stranded nucleic acid molecules (e.g., DNA, RNA, and analogs thereof) that bind a nucleic acid molecule that encodes an CREBRF polypeptide (e.g., antisense molecules, siRNA, shRNA).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat a B cell neoplasia.

The inhibitory nucleic acid molecules of the present invention may be employed as double-stranded RNAs for RNA interference (RNAi)-mediated knock-down of CREBRF expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PAl2, T19-14×, VT-19-17-H2, RCRE, RCRIP, GP+E-86, GP+envAml2, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a CREBRF nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Therapy

Therapy may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly).

Oligonucleotides and other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with a gene listed in Table 2 or 3. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂. NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂) nON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Atm. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Screening Assays

The invention provides cellular compositions (e.g., preadipocytes, adipocytes or precursors of these cell types) comprising a gene whose expression regulates the adipogenesis of the cell. In particular, as reported herein below, the invention provides cells comprising the CREBRF gene that are operably linked to a promoter.

Methods of the invention are useful for the high-throughput, low-cost screening of candidate agents (e.g., inhibitory nucleic acids such as shRNAs, polypeptides, polynucleotides, small organic molecules) that modulate the adipogenesis a cell of the invention. One skilled in the art appreciates that the effects of a candidate agent on a cell is typically compared to a corresponding control cell not contacted with the candidate agent. Thus, the screening methods include comparing the adipogenesis of a cell contacted by a candidate agent to the expression of an untreated reference (i.e., control cell).

In one aspect, the method provides a method of identifying a compound that modulates the expression of a CREB3 Regulatory Factor (CREBRF) polypeptide, comprising: contacting the compound with a nucleic acid that expresses a CREBRF polypeptide under conditions suitable for expression by the nucleic acid; determining the level of expression of the CREBRF polypeptide; determining the level of expression of the nucleic acid in the absence of the compound (i.e., determining the level of expression in a control or reference cell); and comparing the level of expression of the nucleic acid after contact with the compound with the level of expression of the nucleic acid without contact of the compound. Levels of gene expression are determine by methods well-known to those skilled in the art.

In one embodiment, cells of the invention are used to determine potential effects of pharmacological drugs on adipogenesis. The drugs may be proprietary, commercially available or novel compounds and are being administered to patients of various diseases such as diabetes, obesity and cardiovascular diseases. Those that have effects on adipogenesis function in our cell type-specific models would provide entry points for testing drug effects on human adipogenesis, such as changes in weight in patients.

In other embodiments, cells of the invention are used to determine the optimal time for drug administration to a subject. For example, a cell of the invention is contacted with an agent at various time points over the course of the day, and the agent's effect on cell physiology is assayed to determine whether the agent's efficacy or probability of causing adverse side effects alters as a function of the time of administration. The cellular physiology of potential interest in the context of fibroblasts, adipocytes and hepatocytes ranges from RNA and protein production, membrane transport, autophagy and cell division, to cell signaling, cell death, and metabolism. In particular, for example, hepatocytes can be used to study effects of differential temporal application of antidiabetic drugs such as Metformin and TZD, on cellular physiology such as insulin sensitivity, glycogen synthesis and gluconeogenesis, as well as on detoxification and metabolism of xenobiotics.

The effects of agents on a cell's adipogenesis can be assayed by detecting the expression or activity of an adipogenic polypeptide or polynucleotide. Polypeptide or polynucleotide expression can be detected by procedures well known in the art, such as Western blotting, flow cytometry, immunocytochemistry, binding to magnetic and/or antibody-coated beads, in situ hybridization, fluorescence in situ hybridization (FISH), ELISA, microarray analysis, RT-PCR, Northern blotting, or colorimetric assays, such as the Bradford Assay and Lowry Assay.

For example, one or more candidate agents are added at varying concentrations to the culture medium containing a cell of the invention. An agent that modulates the expression of detectable reporter expressed in the cell is considered useful in the invention; such an agent may be used, for example, as an adipogenesis modulator. An agent identified according to a method of the invention is locally or systemically delivered to modulate the adipogenesis of a subject.

In one embodiment, the effect of a candidate agent may be measured at the level of polypeptide production using the same general approach and standard immunological techniques, such as Western blotting or immunoprecipitation with an antibody specific for an adipogenic marker. For example, immunoassays may be used to detect or monitor the expression of protein of interest in a cell of the invention.

Alternatively, or in addition, candidate agents are identified by first assaying those that modulate the marker expression of a cell of the invention and subsequently testing their effect on cells, or on whole animals, which would have implications in human diseases. In one embodiment, an adipogenesis modulator polypeptide is assayed for its ability to interact with adipogenic marker polypeptides, for example, using Gal4 two-hybrid screen as described herein. Such interactions can also be readily assayed using any number of standard binding techniques and functional assays (e.g., those described in Ausubel et al., supra).

### Kits

The invention also provides kits for carrying out the various methods of the invention. For example, in one aspect, such kits are useful for the identification of a CREBRF polymorphism in a biological sample obtained from a subject. In various embodiments, the kit includes one or more probes or primers that identifies a CREBRF nucleic acid sequence encoding a CREBRF polypeptide comprising a glutamine at amino acid position 457 (e.g., an A at nucleotide position 1689), together with instructions for using the primers to genotype a biological sample.

In another aspect, the invention also provides kits for identifying compounds/agents that modulate expression of a a CREBRF. Such kits are useful for the identification of a compound/agent that regulates adipogenesis in a subject. In various embodiments, the kit includes cells of the invention comprising the CREBRF gene that is operably linked to a promoter, together with instruction for using the cells to identify a modulator.

In one embodiment, the instructions include instructions using the kits in accordance with the methods of invention. In certain embodiment, the instructions include at least one of the following: description of a therapeutic agent (e.g., for treatment of obesity or symptoms thereof); dosage schedule; administration precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

In some embodiments, the kit comprises a sterile container which contains composition of the invention; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Materials and Methods

The experimental examples below were performed using the following materials and methods.

### Participants.

The participants in this study are derived from the populations of the Independent State of Samoa and the United States territory of American Samoa. Two samples were used in this study: a discovery sample of 3,072 phenotyped and genotyped Samoans and a replication sample of an additional 2,583 phenotyped and genotyped Samoans and American Samoans. The parent GWAS study, sample selection and data collection methods, and phenotype levels, including lipids and lipoproteins have been reported³. The discovery GWAS data set will be available in dbGAP (access number: phs000914). This study has been approved by the Health Research Committee of the Samoa Ministry of Health and the Institutional Review Boards of Brown University, University of Cincinnati, and University of Pittsburgh. All participants gave informed consent.

The discovery sample is drawn from 3,475 men and women (n=1,437 male), ages 24.5 to <65 years who reported Samoan ancestry (based on having four Samoan grandparents). Recruitment took place between February and July 2010 in 33 villages across both islands (Upolu and Savaii) of Samoa³. A population-based design was employed and consenting participants completed interviews targeting lifestyle factors related to cardiometabolic health (health history, socio-economic position, dietary intake, and physical activity) and anthropometric measurements (height, weight, blood pressure, body composition), and gave fasting whole blood samples for biochemical and genetic assays. A description of the prevalence of non-communicable diseases and associated risk factors is provided in Hawley et al.³.

In the original GWAS study design, the discovery sample size goal of 2,500 (which is exceeded) was chosen so as to have high power to detect risk SNPs with realistic effect sizes. Power was estimated as follows: Quanto^{34,35} was used to estimate the power to detect the FTO rs9930506 SNP, which in the Sardinia study³⁶ explained 1.34% of the BMI variance. If it is assumed that the SNP has the same allele frequencies and BMI has the same overall mean and standard deviation as in Scuteri et al³⁶, then at a significance level of 1×10⁻⁵, power >80% when the risk SNP explains at least 1.1% of the variance (and power of 90% when the SNP explains 1.3% of the variance). If it is instead tested at 1×10⁻⁷, power is >80% if the SNP explains at least 1.5% of the variance.

The replication sample consists of individuals from two samples of Samoans studied in 1990-95 and in 2002-03, which were analyzed as if all samples were unrelated because genome-wide marker data was not available. The 1990-95 study sample derives from a longitudinal study of adiposity and cardiovascular disease risk factors among adults from the U.S. territory of American Samoa and the independent nation of Samoa. Although there is substantial economic disparity between the two polities, the Samoans from both territories form a single socio-cultural unit with frequent exchange of mates. Genetically they represent a single homogenous population^{37,38}. Participants were between 25-55 years of age at baseline and reported that all four grandparents were of Samoan ancestry. Detailed descriptions of the sampling and recruitment were reported previously³⁹⁻⁴¹. Briefly, participants were recruited from 46 villages and worksites in American Samoa in 1990 and nine villages in (then Western) Samoa in 1991. All participants were free of self-reported history of heart disease, hypertension, or diabetes at baseline. There were 413 and 607 genotyped and phenotyped individuals available from American Samoa in 1990 and from Samoa in 1991, respectively (Table 1). Due to lack of genome-wide marker data on these samples, relatedness cannot be inferred, and so these were treated as unrelated in the analyses.

The 2002-03 family study sample includes adults and children recruited as part of an extended family-based genetic linkage analysis of cardiometabolic traits^{1,42-45}. Probands and relatives were unselected for obesity or related phenotypes. The recruitment process and criteria used for inclusion in this study are described in detail previously^{42,45}. There were 590 adults, 18-89 years, from 2002 in American Samoa; and 493 adults, 19-82 years, and 409 children ages 5-<18 years, from 2003 in Samoa, available with genotypes and phenotypes (Table 1). The analyses of these samples were adjusted for relatedness using kinships derived from the known family structures (which had been verified to be consistent with relatedness estimates derived using genome-wide microsatellite markers)¹.

### Anthropometric/Biochemical Measurements.

Height, weight and BMI were measured as previously describee^{39,46}. Polynesian cutoffs were used to classify individuals as normal weight, overweight or obese based on BMI of <26 kg/m², 26-32 kg/m², and >32 kg/m² respectively². Obesity in children was categorized from BMI using the international age and sex-specific classifications developed by Cole et al.⁴⁷

In the discovery sample, hypertension and abdominal (at the level of the umbilicus) and hip circumferences were measured in duplicate and averaged (Table 4). Bioelectrical impedance measures of resistance and reactance (RJL BIA-101Q device, RJL Systems, MI, USA) were used to estimate percent body fat based on Polynesian-specific equations^{2,46}. Serum separated from whole blood samples, collected after a 10-hour overnight fast was assayed for cholesterol (total, HDL and LDL), triglycerides, glucose, and insulin. The assay techniques for these metabolic markers have been described previously′. Individuals were classified as having type 2 diabetes based on a fasting serum glucose >126 mg/dL or the current use of diabetes medication⁴⁸. Hypertensives either had a systolic BP >140 mm Hg or diastolic BP >90 mm Hg, or were currently taking hypertension medication. Additionally, serum levels of leptin and adiponectin were obtained by using commercially available radioimmunoassay kits (EMD Millipore Inc., St. Charles, Mo., USA). HOMA-IR was calculated as glucose (mg/dL)×insulin (μU/mL)/405 as recommended.⁴⁹

### Genotyping.

DNA was extracted from whole blood as previously reportee⁴². In the discovery sample, genotyping was attempted on 3,298 DNA samples (including 3,194 participants, 34 duplicates and 70 positive controls) across 909,622 probes using a Genome-Wide Human SNP Array 6.0 (Affymetrix, California, USA). Genotyping of the discovery samples was performed on 96 well plates, each plate containing two reference samples: 1) REF103 provided by Affymetrix, and 2) a Coriell DNA sample, NA15510, and a negative control. A duplicate sample from the same plate was introduced in each plate with blinded IDs for the laboratory personnel. The samples were not randomized and were processed in the order collected in the field. Laboratory personnel were blind to the sample phenotypes.

Extensive quality control was conducted based on a pipeline developed by Laurie et al.⁵⁰ including assessment of probe and sample quality (probes and samples excluded with missingness rates >5%), sex validation, investigation of genotyping batch effects, assessment of cryptic relatedness and population substructure, and duplicate sample and duplicate probe discordance. Of the 3,194 samples attempted for genotyping, 4 were dropped due to high genotyping missingness, 3 due to discrepancy between reported and apparent genetic gender, 7 due to apparent sex chromosome aneuploidy, 9 due to chromosomal abnormalities such as deletions and duplications, 2 due to apparent sample admixture, and 50 due to poor cluster resolution across the genome. After quality control, 3,119 samples genotyped for 895,103 unique markers were available to conduct genome-wide association studies. An additional 25 participants were excluded due to self-reported pregnancy and 3 because each is one of a pair of monozygotic twins. There were 19 participants missing BMI. Complete phenotype and genotype data were available for up to 3,072 participants.

To test for possible overlap between the samples from our three studies, 116 single-nucleotide polymorphisms (SNPs) genotyped were used in common across all our samples. These 116 SNPs, including rs12513649, were chosen based on their association signals for a whole suite of traits in the discovery sample. At loci with multiple significant SNPs, the peak SNP was chosen as representative of that locus. At loci (defined as 1 Mbp windows) with different peak SNPs for different phenotypes, the SNP with the smallest P value among the associated phenotypes was genotyped as representative of that locus. These SNPs spanned all autosomal chromosomes and the X chromosome, and were at least 1 Mb away from each other and not in linkage disequilibrium with each other (r2<0.3 for all but one pair of adjacent markers; r2=0.73 between rs4932738 and rs7252689 on chromosome 19). Genotyping of variants selected for validation (described below) in the replication sample was performed using custom-designed TaqMan® OpenArray Real-Time PCR assays (Applied Biosystems). SNPs that could not be genotyped using OpenArray assays, including rs12513649, were genotyped individually using TaqMan® SNP Genotyping assays (Applied Biosystems). For replication genotyping, in each 384 well plate (n=8), 4 duplicates from the same plate with blinded ID were included; each plate also contained 8 negative controls and 8 Coriell samples (NA15510). The quality of genotype clustering for each SNP was verified and corrected manually. Eight subjects could not be genotyped due to technical difficulties.

### Statistical Analysis: Genotyping Data.

During quality control, significant relatedness was observed among the discovery sample participants, so empirical kinship coefficients were estimated using the genotyped markers, in two iterations. In the first iteration, 10,000 independent autosomal markers were selected using PLINK⁵¹ to generate empirical kinship coefficients using GenABEL⁵². Individuals with kinship coefficients less than 0.0625 (first-cousin) were considered unrelated. A maximal set of 1,891 unrelated individuals was determined using previously published methods⁵³. In the second iteration, the kinship matrix between all participants was estimated using, a new set of 10,000 independent autosomal markers that had been selected using the set of unrelated individuals.

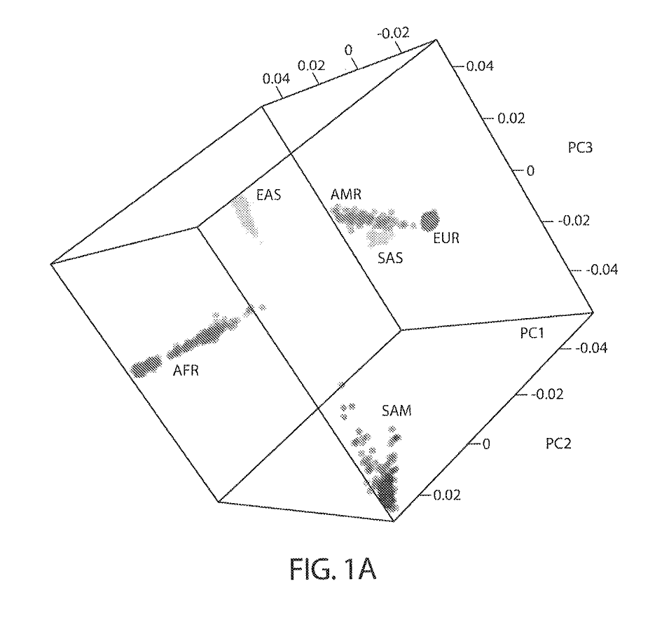

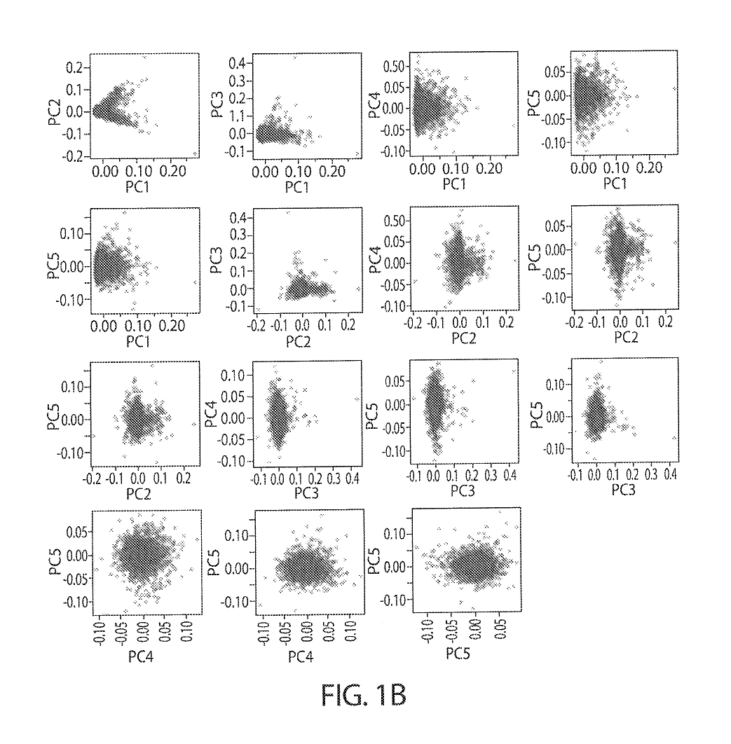

The genetic ancestry of our discovery sample, where every individual self-reported having four Samoan grandparents, was assayed via principal components analyses using PCAiR⁵⁴. We conducted two principal components analyses. Firstly, to examine the relationship of the Samoans against other continent populations, we compared the genotypes of a randomly chosen subset of 250 Samoans against genotypes from individuals comprising HapMap Phase 3. Genotype management was performed using PLINK⁵¹. HapMap Phase 3 genotypes⁵⁵ were merged with the genotypes from the Samoan discovery sample. SNPs with a minor allele frequency <0.05, with a missingness rate >0.1, and located within regions problematic for the calculation of principal components analysis (the major histocompatibility locus on 6p21, the region near LCT on 2q21, and common inversion regions on 8p23 and 17q21) were dropped. Markers were further pruned down to every fourth marker. The PC-AiR algorithm was applied to the remaining 111,438 markers: the PCs were estimated in the unrelated subjects as determined by the KING-robust kinship coefficient estimator⁵⁶ and extended to relatives in the dataset based on their genetic similarity. The first three principal components from this analysis are shown in FIG. 1A. Secondly, to examine the potential for population stratification within the Samoans, we calculated principal components within the Samoan participants in our sample. SNPs were again removed based on the same minor allele frequencies, missingness rates and location within problematic regions as above. Markers were pruned based on linkage disequilibrium down to a set of independent SNPs, and the PC-AiR algorithm was applied to the remaining 72,586 markers. The first six principal components from this analysis are shown in FIG. 1B. Note that the between-population ‘distances’ shown in Supplementary FIG. 1A should be interpreted with caution, as we did not correct for how SNPs were selected to be on the Affymetrix genotyping array⁵⁷. Correcting for SNP ascertainment bias in a well-calibrated manner requires not only sophisticated and careful modeling of the ascertainment process but also requires sequencing data (which is not available yet) to validate that the correction method works correctly⁵⁸.

BMI was log-transformed to approximate normality. Residuals were generated by linear regression against age, age², sex and the interactions between age and sex. Association between autosomal marker genotypes and the BMI residuals was tested while using the empirical kinship matrix to adjust for subject relatedness. Note that population substructure is accounted for in our analyses by inclusion of the empirical kinship model in the analysis models, because, as Hofmann⁵⁹ states “explicitly modeling the pairwise relatedness between all individuals captures both population structure and kinship”. The tests were conducted using a score test using the mmscore function in GenABEL⁶⁰. The statistics between X chromosome genotypes and BMI residuals were calculated in GenABEL without adjusting for the empirical kinship estimates. Following analysis, 230,554 SNPs with a minor allele frequency <0.01 (including 23,612 monomorphic SNPs) and then 4,093 SNPs with HWE test p values <0.00005 were filtered out, resulting in 659,492 autosomal and X-linked SNPs used for analyses. Inflation due to population stratification and cryptic relatedness was assessed by estimating λ_{GC} using the lower 90% of the p value distribution⁶¹.

Genome-wide significance for GWAS p values (p_{G}) was set at p_{G}<5×10⁻⁸. Suggestive association was set at p_{G}<10⁻⁵. Statistical power to detect signals at these thresholds was calculated using the Genetic Power Calculator (Purcell, S., Cherny, S. S. & Sham, P. C. Genetic Power Calculator: design of linkage and association genetic mapping studies of complex traits. Bioinformatics 19, 149-150 (2003)). Probe intensities plots for each significantly and suggestively associated SNP were examined for genotype-calling errors.

SNPs with p_{G}<10⁻⁵ were chosen for association validation in the additional Samoan participants from the replication sample. At loci (defined as a 1 Mbp window) with multiple SNPs more significant than this threshold, the peak SNP was chosen as representative of that locus.

The replication sample was divided into three groups for analysis: the 1990-1995 study participants, the 2002-03 family study adults (age ≥18), and the 2002-03 family study children (age <18). For the purposes of the meta-analyses, the studies were not further subdivide by nation; doing so would have broken up pedigrees in the family study that span both nations. For consistency, the 1990-1995 study was therefore not subdivided by nation either. All samples, including those from the discovery sample, were examined using the 116 SNPs typed in common across all samples (validation genotypes) for genetic identity that might have arisen through recruitment into multiple studies over the two decades that they span. One sample of each pair that had an estimated identity-by-descent >0.9 as estimated in PLINK were removed from analysis. For the participants, both adults and children, from the 2002-03 family study, kinship coefficients were calculated from the recorded pedigrees using the kinship2 package⁶² in R⁶³. Replication association analyses were performed using GenABEL⁵² for each group, using the kinship coefficients to adjust for relatedness in the family sample. There are no sufficient marker data to infer relatedness and adjust for it in the 1990-1995 study, so they were treated as unrelated. The same covariates used in the GWAS analysis were used in the replication regression models, with an additional variable indicating whether subjects were from Samoa or American Samoa. Prior to meta-analysis, quality control of the summary statistics was performed using EasyQC⁶⁴ to check for strand and allele frequency consistency. Meta-analysis was performed using METAL⁶⁵ to generate two replication p values: one for the replication sample and one for the replication sample and discovery sample together (Table 2). The p-value-based method was used with sample sizes as weights with genomic control correction turned off. Heterogeneity across all the cohorts were assessed by calculating both Cochrane's Q and the I² statistic⁶⁶⁻⁶⁸.

### Targeted Sequencing.

Before undertaking targeted sequencing, we first used SHAPEIT⁶⁹⁻⁷³ and IMPUTE2⁷⁴⁻⁷⁶ to impute in our region of interest centered on rs12513649 using the December 2013 1,000 Genomes Phase I integrated variant set release haplotype reference panel. It implicated only one strongly-associated variant (with a predicted allele frequency of 0.075), but when we genotyped it in a pilot sample, it turned out to be monomorphic (as it was in the subsequent targeted sequencing experiment described below). Based on this experience, as well as on what we would expect given the unique population history of the Samoans, we believe that the best way to do accurate imputation in the Samoans is by using a Samoan-specific reference panel. This concurs with recent recommendations for optimal fine-mapping in populations with unique ancestries not found in the cosmopolitan reference panel⁷⁷. A panel of 444 of our Samoans from the discovery sample is currently being whole-genome sequenced by the NHLBI TOPMed Consortium.

A 1.5 Mbp segment (NC_000005.09:171583933_173083933) around rs12513649 was chosen for targeted sequencing by finding the boundaries of the linkage disequilibrium block containing rs12513649. This block was defined by multiallelic D′ lows (calculated using gPLINK) within 2 Mbp of rs12513649 and extended from rs1433019 to rs4868246. The targeted region was then extended from these points until it encompassed 1.5 Mbp. Sequencing was performed on 96 discovery sample participants optimally chosen using INFOSTIP⁷⁸. The sample size of 96 was chosen due to fiscal constraints, and was estimated to recover 94% of the information had we been able to sequence everyone. Baits were derived using SureDesign (Agilent Technologies), with additional baits derived based on blat analysis. DNA libraries were prepared using SureSelect (Agilent Technologies) and sequenced using 100 bp paired-end runs on an Illumina HiSeq 2500 with the goal that at least 95% of the targeted region achieves a coverage depth of 20× or greater. Mean bait coverage was 81×. Samples were processed using BWA, GATK3 (QD<2.0, MQ<40.0, FS>60.0, MQRankSum<-12.5, ReadPosRankSum<-8.0), and HaplotypeCaller with hard cutoffs. This resulted in 99.6% concordance to VeraCode array calls, and 98.35% of single nucleotide variants were in dbSNP 138.

### Targeted Sequencing: Library Preparation and Exome Sequence Capture.

DNA fragmentation was performed on 200 ng of genomic DNA using a Covaris E210 system, which shears DNA to fragments 150 to 200 bp in length with 3′ or 5′ overhangs. End repair was performed where 3′ to 5′ exonuclease activity of enzymes removes 3′ overhangs and the polymerase activity fills in the 5′ overhangs. An ‘A’ base is then added to the 3′ end of the blunt phosphorylated DNA fragments which prepares the DNA fragments for ligation to the sequencing adapters, which have a single ‘T’ base overhang at their 3′ end. Ligated fragments are subsequently size selected through purification using SPRI beads and undergo PCR amplification techniques to prepare the ‘libraries’. The Caliper LabChip GX is used for quality control of the libraries to ensure adequate concentration and appropriate fragment size.

Exon capture was done using the Agilent SureSelect Human All Exon Target Enrichment system, which results in ˜51 Mb of targeted sequence capture per sample. Under standard procedures, biotinylated RNA oligonucleotides were hybridized with 500 ng of the library. Magnetic bead selection is used to capture the resulting RNA-DNA hybrids. RNA is digested and remaining DNA capture PCR-amplified. Sample indexing is introduced at this step. The Agilent Bioanalyzer (HiSensitivity) is used for quality control of adequate fragment sizing and quantity of DNA capture.

### DNA Sequencing.

DNA sequencing was performed on an Illumina® HiSeq 2500 instrument using standard protocols for a 100 bp paired-end run. Six samples were run per flowcell, guaranteeing >90-95% completeness at a minimum of 20× coverage.

### Targeted Sequencing: Variant Calling.

Illumina HiSeq reads was processed through Illumina's Real-Time Analysis (RTA) software generating base calls and corresponding base call quality scores. Resulting data was aligned to a reference genome with the Burrows-Wheeler Alignment (BWA) tool (Li, H. & Durbin, R. Fast and accurate short read alignment with Burrows-Wheeler transform. Bioinformatics 25, 1754-1760 (2009)). resulting in a SAM/BAM file. Post processing of the aligned data included local realignment around indels, base call quality score recalibration performed by the Genome Analysis Tool Kit (GATK) (McKenna, A. et al. The Genome Analysis Toolkit: a MapReduce framework for analyzing next-generation DNA sequencing data. Genome Res 20, 1297-1303 (2010)) and flagging of molecular/optical duplicates using software from the Picard program suite. Per-sample and multi-sample variant calling was performed by GATK Haplotype Caller. Per sample data quality metrics include (but are not limited to) transition/transversion ratios (ts/tv), percent in dbSNP, concordance and heterozygote sensitivity with previously generated genotyping data, capture specificity and percent of targeted bases covered=>20×.

### Imputation.

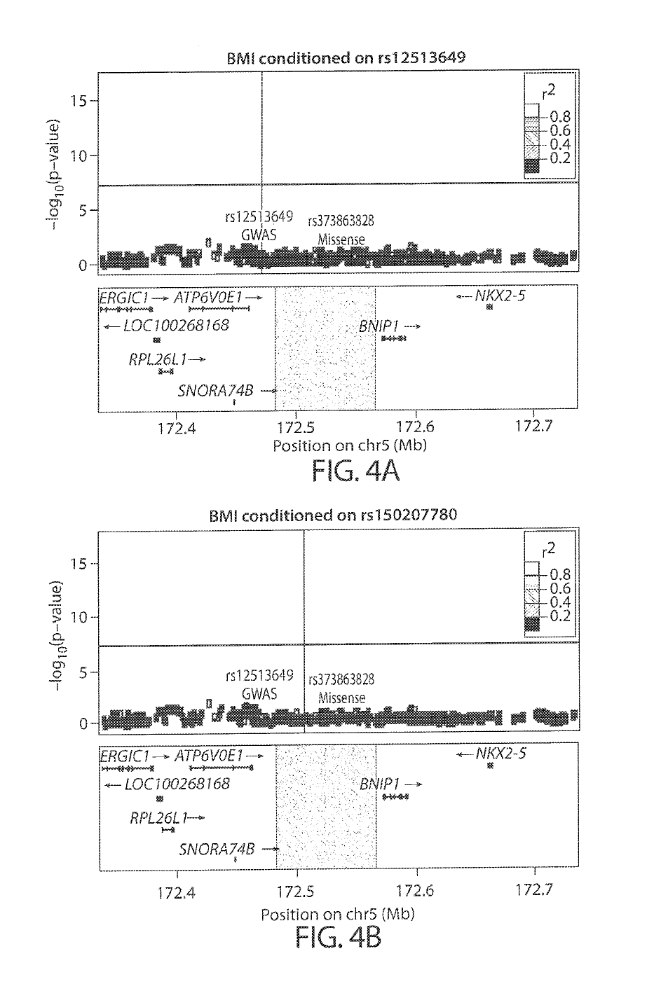

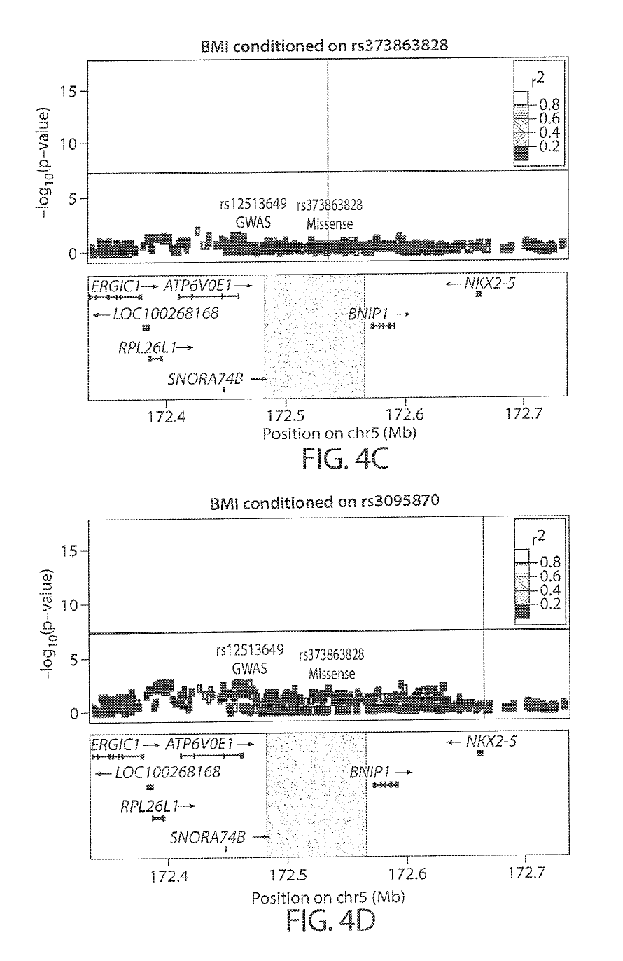

The targeted sequencing sample was prephased using SHAPEIT⁶⁹⁻⁷³, and then imputed into our discovery sample using IMPUTE2⁷⁴⁻⁷⁶. Association testing was carried out using ProbABEL⁷⁹, adjusting for relatedness with the empirical kinship matrix generated by GenABEL. Three variants had nearly equivalent P values (rs12513649, rs150207780, rs373863828) due to nearly perfect linkage disequilibrium between them (r2>0.988); rs150207780 and rs373863828 were imputed very well (IMPUTE2 info metric=0.954 for both variants). To determine which of these variants might be the most likely causal candidate, we tested for association in the targeted sequencing region conditioned on each of these variants as well as the next most significant variant (rs3095870; info metric=0.957), using ProbABEL and adjusting for relatedness. As expected for variants in such high LD, the signals in the region were eliminated after conditioning (FIG. 4).

### Bayesian Fine-Mapping.

For fine-mapping using the imputed variants, 160 variants were selected with minor allele frequency >0.05 on either side of the missense variant rs373863828. These 321 SNPs spanned from 172368674 to 172670745 on chromosome 5, including from the GWAS variant rs12513649 on the left to the variants with significant P values near NKX2-5 on the right (FIG. 1b). The PAINTOR program¹³ was then used to estimate posterior probabilities of causality for each variant in the region, based on Z scores derived from the ProbABEL estimates described above and the linkage disequilibrium correlation matrix as estimated by the R package ‘snpStats’^{m}. We used the default maximal number of causal variants of 2 and the default number of maximumiterations of 10. We also used PAINTOR to incorporate prior information about coding and regulatory DNA regions using the genome segmentation data derived by the ENCODE project⁸¹. This annotation segments the genome into seven classes: 1) CTCF enriched element, 2) Predicted enhancer, 3) Predicted promoter flanking region, 4) Predicted repressed or low activity region, 5) Predicted transcribed region, 6) Predicted promoter region including transcription start site, and 7) Predicted weak enhancer or open chromatin cis regulatory region. PAINTOR was run using these segmentations in each of the six ENCODE cell lines, and then the most significant annotation (a predicted transcribed region in the HepG2 liver carcinoma cell line) was used when estimating the posterior probabilities. The ‘combined’ ENCODE genomic segmentation annotation was downloaded from the ftp.ebi.ac.uk/pub/software/ensembl/encode/supplementary/integration_data_jan2011/byD ataType/segmentations/jan2011/hub site.

Confirmatory genotyping.

Genotyping was attempted for both rs150207780 and rs373863828 using TaqMan® in all discovery and replication sample participants. The assay for rs150207780 failed; genotyping was not reattempted because it showed no residual association signal in the analyses of the imputed data conditioned on missense variant rs373863828 (FIG. 4). The replication plates included the 96 samples that had been sequenced in the targeted sequencing experiment. Laboratory personnel were blind to the sequence-derived genotypes of these 96 samples, as well as to phenotypes of all the samples. Association analysis was performed using the same regression models and meta-analysis as the GWAS and replication analyses above. Effect size estimates were made using untransformed BMI separately in the men and women of the discovery sample with age and age² as covariates.

### Association Analyses of Additional Phenotypes.

rs373863828 genotypes examined for association with the additional adiposity-related phenotypes listed in Table 4. Association was assessed in both the discovery sample (Table 4 and Table 5a) and in a mega-analysis of the replication sample adults (Table 5b). While meta analysis of properly transformed phenotypes generates more accurate pvalues (as we did in Table 2), we chose instead here to carry out mega analyses because we are primarily interested in estimating the effect sizes on the traits' natural scales. Sexstratified analyses were also conducted in both samples (Table 5). Diabetics were excluded from analyses of glucose, insulin and HOMA-IR. Since the distributions of leptin varied greatly between women and men, each sex was analyzed separately for this trait. Residuals for quantitative traits were generated using linear regression; for qualitative traits logistic regression was used. Age, age², sex and the interactions between age and sex and age² and sex were included in all models initially. For glucose, insulin, HOMA-IR, adiponectin, leptin, and diabetes status, second sets of residuals were generated including log-transformed BMI as a covariate. Sex and age-sex interactions were not included in the sex-stratified models. In the replication mega-analysis models, polity (Samoa or American Samoa) and cohort (1990s or 2000s) were included in the models initially as well. Stepwise regression was used to reduce the number of covariates for each trait separately. For quantitative traits, Residuals were tested for association using the mmscore function of GenABEL⁵², adjusted for the empirical kinship matrix as above. Dichotomous traits were analyzed using the palogist function of ProbABEL⁷⁹ while adjusting for covariates and empirical kinship. A Bonferroni-corrected p value threshold of 0.0033 was used to assess significance; this is conservative as it adjusts for 23 tests even though some of traits are correlated with each other. To assess a possible survivor effect as the cause of the association between the BMI-increasing allele and decreased fasting glucose levels and risk of diabetes, we conducted linear regression of age by genotype. In the discovery sample, regarding the association of rs373863828 with BMI, fasting glucose, fasting insulin, obesity risk, and diabetes risk, the addition of the first 10 ‘local’ principal components from Supplementary FIG. 1b into the statistical models has a negligible effect on the effect estimates and the statistical significance (results not shown).

### Expression of CREBRF in Human and Mouse Tissues.

For human gene expression analysis, a Human Normal cDNA Array was obtained from Origene (Cat#HMRT103 and HBRT101). The human standard curve was prepared from Control Human Total RNA (ThermoFisher Scientific, 4307281). For mouse gene expression analysis, mouse tissues were collected between 8-10 am from littermate-matched, from ad lib-fed, male C56BL/6J mice at 10 weeks of age (n=6/group). The mouse standard curve was prepared from pooled kidney RNA from the above mice. mRNA was prepared using the RNeasy Lipid Tissue Mini Kit with on-column DNase treatment (Qiagen) followed by reverse transcription to cDNA using qScript cDNA Supermix (Quanta Biosciences). Gene expression was determined by qPCR (Quanta PerfCTa SYBR Green FastMix or PerfeCTa qPCR FastMix) using an Eppendorf Realplex System. Human CREBRF was amplified using the following CREBRF-specific primers: forward 5′ ATGTATGAACTGGATAGAGAGATG, reverse 5′ GTTAGGTCTTCACAGTATGTATCC. Mouse Crebrf was amplified using a Crebrf-specific primer-probe set (ThermoFisher ScientificCat# Mm00661539_ml). CREBRF expression was normalized to species-specific peptidylprolyl isomerase A/Cyclophilin A as the endogenous control gene (ThermoFisherScientific 4333763T and Mm02342430_gl for human and mouse, respectively). Mouse data are expressed as mean plus s.e.m. Data are relative expression values, and so randomization, blinding, and statistical comparisons were not indicated. Gene expression analysis was performed in accordance with Minimum Information for Publication of Quantitative Real-Time PCR Experiments (MIQE) guidelines. Animal experiments were approved by the University of Pittsburgh Institutional Animal Care and Use Committee and conducted in conformity with the Public Health Service Policy for Care and Use of Laboratory Animals. Human samples from Origene Technologies conform to Federal Policies for protection of human subjects (45 CDR 46) and are HIPPA compliant. Additional information and documentation can be obtained by contacting the company.

### Plasmid Construction and Mutagenesis.

Expression plasmids with the eGFP and human CREBRF (NM_153607.2) open reading frames were obtained from GeneCopoeia (EX-EGFP-M10, EXEX-E3374-M10; Rockville, Md., USA). The backbone vector was pReceiver-M10, which had a cytomegalovirus promoter and a carboxy-terminal Myc-(His)₆ tag. A rare missense variant, c.1447A>G, p.Thr483Ala (rs17854147), affecting a conserved residue was present in CREBRF open reading frame, which was predicted to be a loss-of-function variant. To avoid using this potentially function-altering variant, the variant sequence was converted to wild-type CREBRF and the BMI risk allele, c.1370G>A, p.Arg457Gln, (rs373863828), was introduced using PCR mutagenesis. The segments obtained by PCR in each plasmids were verified by sequencing before large-scale plasmid purification for transfection.

### Cell Culture and Transfection.

The mouse embryonic fibroblast cell line 3T3-L1 was obtained from ATCC (Manassas, Va., USA). No genetic authentication has been performed. However, the phenotype of the cells is consistent with previous publications. Cells were maintained in Dulbecco's modified Eagle's medium (DMEM; Gibco, Grand Island, N.Y.) supplemented with 10% newborn calf serum (NCS; Sigma, St. Louis, Mo.), 100 units/mL penicillin and 100 μg/mL stremptomycin (Sigma), 3.7 g/L NaHCO₃, 4.77 g/L HEPES in a 37° C. with 5% CO2 humidified incubator. 3T3-L1 preadipocytes were transfected with plasmids containing eGFP-only negative control, wild-type human CREBRF, or the p.Arg457Gln variant using Lipofectamine 2000 (ThemoFisher Scientific, Waltham, Mass.) in triplicates. Transfected cells were kept under selection with 500 μg/mL Geneticin (G418, ThemoFisher Scientific) for 3 weeks to generate stable cell lines. Mycoplasma testing was performed by PCR and DAPI staining. All cells used in this study tested negative.

### Adipocyte Differentiation.

The differentiation of 3T3-L1 to adipocytes was carried out as described previously⁸². Differentiation was induced 2 days post confluence with a differentiation cocktail including 3-isobutyl-1-methylxanthine (IMBX, 0.5 mM; Sigma), dexamethasone (0.25 μM; Sigma), human insulin (1 μg/mL; Sigma) in basic media with 10% fetal bovine serum (FBS). After 2 days, the media was replaced with maintenance media with 10% FBS and 1 μg/mL human insulin. After further 2 days, the maintenance media was replaced with growth media containing 10% FBS, 100 units/mL penicillin and 100 μg/mL stremptomycin (Sigma) and was changed every other day for up to 10 days. Geneticin (500 μg/mL) selection was maintained throughout the differentiation protocol for stable transfected cells.

Oil Red O plate assay.

Oil Red O Staining has been established as a useful tool to measure intracellular triglyceride accumulation⁸³, a quantitative measure of adipocyte differentiation. Cells were seeded in 96-well cell culture plates at 10,000 cells/well with 8 technical replicates. At endpoints of interest, cells were fixed with 4% paraformaldehyde for 15 min. Stock solution was 0.3% Oil Red O solution that was prepared from Oil Red O solution purchased from (Sigma, O1391). Working solution contained stock solution and water with the ratio of 24:16 v/v. After fixation, cells were rinsed with PBS and incubated with oil red O working solution for 15 min (30 μL per well). Washing with PBS three times was performed to remove residual oil red O solution. Then, 100 μL isopropanol was added in each well to elute the dye and the absorbance was measured at 560 nm. Cells containing media only served as blanks. Blank values were subtracted from experimental samples. Cells in a parallel plate were lysed using CelLytic M (Sigma) and the protein concentration was measured using the Bradford assay⁸⁴ (Bio-Rad, Hercules, Calif.). Absorbance data were normalized to protein concentration and expressed in OD₅₆₀/μg units.

### Oil Red O Staining and Microscopy.

To visualize lipid accumulation, cells were cultured on coverslips. Eight days after confluence the media was removed and the cells were washed twice with PBS. Fixation in 4% paraformaldehyde for 10 minutes at room temperature was followed by staining with Oil Red O working solution for 30 minutes at room temperature. The Oil Red O solution was aspirated and the cells were rinsed 6 times in distilled water. The cells were counterstained with hematoxylin for 5 minutes at room temperature followed by rinsing 6 times with distilled water. The coverslips were mounted with glycerol-gelatin media and images were captured using a DM5000 (Leica Microsystems, Buffalo Grove, Ill.) photomicroscope.

### Triglyceride Assay.

Cells were harvested 8 days after confluence and the PicoProbe Triglyceride Quantification Assay Kit (Abeam, ab178780) was used to measure the level of triglycerides in cell lysate. The triglyceride level (pmol) was normalized to the amount of protein measured by the Bradford method⁸⁴ in each lysate sample.

### Bioenergetic Profiling.

Oxygen consumption rate (OCR), a measure of mitochondrial respiration, and extracellular acidification rate (ECAR), a measure of glycolysis, were determined using an XF96 extracellular flux analyzer (Seahorse Bioscience, North Billerica, Mass.). Transfected 3T3-L1 cells were seeded in a 96-well XF96 cell culture microplate (Seahorse Bioscience) at a density of 7000 cells per well in 200 μL DMEM (4.5 g/L glucose) supplemented with 10% FBS (Sigma) 36 hours before the measurement. Six replicates per cell type were included in the experiments and four wells were chosen evenly in the plate to correct for temperature variation. On the day of assay, the growth media was changed with assay media (unbuffered DMEM with 4.5 g/L glucose). Oligomycin at a final concentration of 2.0 μM, FCCP (carbonyl cyanide-p-trifluoromethoxyphenylhydrazone) at 1.0 μM, 2-deoxyglucose at 100 mM and rotenone at 15.0 μM were sequentially injected into each well in accordance with the manufacturer's protocol. Basal mitochondrial respiration, maximal respiration, ATP production and basal glycolysis were determined according to the manufacturer's instructions. At the conclusion of the assay cells in the analysis plate were lysed using CelLytic M (Sigma), the protein concentration was measured using the Bradford assay⁵⁹ (Bio-Rad, Hercules, Calif.) and used to normalize the bioenergetic profile data.

### Quantitative RT-PCR.

Total RNA was harvested using an RNeasy Mini Kit (Qiagen) and cDNA was generated using the Superscirpt III Reverse Transcriptase (ThemoFisher Scientific). Quantitative RT-PCR analysis used SYBR Green PCR Master Mix (BioRad) with primers for human CREBRF (5′-GAAGACCTGAAGGAGGTGACT and 5′-GTTCCACTCA GATGGTCTCA GC), mouse Crebrf (5′-GAGGACTTGAAGGAGATGACG and 5′-CAGAAGGCCTCAGAATCCTC), mouse), mouse Pparg2 (5′-CCAGAGCATGGTGCCTTCGCT and 5′-CAGCAACCATTGGGTCAG), mouse Cebpa (5′-CAAGAACAGCAACGAGTACCG and 5′-GTCACTGGTCAACTCCAGCAC), mouse beta actin (Actb, 5′-CCACTGCCGCATCCTCTTCC and 5′-CTCGTTGCCAATAGTGATGACCTG). Samples were run on a QuantStudio 12 Flex Real Time PCR System (ThemoFisher Scientific). The efficiency of the qPCR assays was determined using a template dilution series and was found to be ≥0.9. The results were analyzed using ExpressionSuite Software v1.0.4 either using the ΔΔCt method⁸⁵, or by calculating the 2e^{*Δct} value, where e is PCR efficiency and ΔCt is the threshold cycle difference between the target gene and beta actin (Actb) as a reference gene.

### Starvation and Rapamycin Stimulation

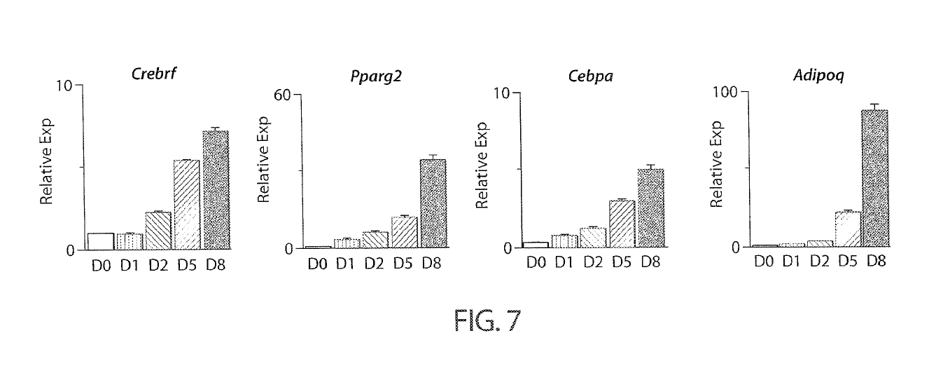

3T3-L1 preadipocytes were subjected to starvation for 2 hours, 4 hours, 12 hours, and 24 hours by culturing cells in Hank's Balanced Salt Solution (HBSS; Gibco, Grand Island, N.Y.). To investigate the response to refeeding starving cells, cells undergoing 12 hours starvation were fed with fresh growth medium for an additional 12 hours (“24 hR” in FIG. 7A). For rapamycin stimulation, preadipocytes were treated with 20 ng/ml rapamycin (Sigma), for 2 hours, 4 hours, 12 hours and 24 hours. A set of cells kept in rapamycin for 12 hours were cultured in fresh growth medium for the following 12 hours (“24 hR” in FIG. 7B). To quantify cell survival, 3T3-L1 cells and transfected cells were seeded in 6-well plates with at 86000 cell per well. Two days later, the cells were starved in HBSS. At 2 hours, 4 hours, 6 hours, 12 hours and 24 hours, cells were collected and 100 μL cell suspension samples were added to equal volume of trypan blue (Life Technologies). The mixture was loaded in an automated cell counter (Cellometer Mini, Nexcelom Bioscience) and viable cell numbers were measured. Cell death rates were calculated by subtracting the number of viable cells at 6 hours from cell numbers at 0 h and dividing the result by 6 hours.

### Cell Studies Statistical Analysis: Gene Expression, Oil Red O Plate Assay and Bioenergetic Profile and Cell Count Data.

For cell studies, adequate sample sizes were determined based on publications using similar methods and pilot experiments. No blinding was done. Each experiment was performed twice with similar results unless otherwise stated in the figure legends. The data were initially evaluated by one-way ANOVA implemented in SPSS (IBM, Armonk, N.Y.). Homogeneity of variances was examined using the Levene's test. Two-sided Bonferroni and Games-Howell post hoc tests were used to compare data with equal and unequal variance, respectively. Alternatively, pairwise t-tests were used. A p-value less than 0.05 was considered to be statistical significance. SPSS analyses were verified using the same tests implemented in the statistical programming language R⁶³ (R Foundation, Vienna, Austria).

Selection analyses.

Based on the genome-wide Affymetrix 6.0 SNP genotype data, we used Primus^{86,87} to select 626 individuals from the discovery sample using a kinship threshold (0.039) halfway between first and second cousins, so that first cousins and more closely related relatives were excluded. These ‘unrelated’ individuals were then haplotyped using SHAPEIT⁶⁹⁻⁷³, and were annotated with ancestral allele information using the selectionTools pipeline⁸⁸. Haplotype bifurcation diagrams and extended haplotype homozygosity (EHH) plots were drawn using the ‘rehh’ R package⁸⁹. The haplotype bifurcation diagram⁹⁰ visualizes the breakdown of linkage disequilibrium as one moves away from the core allele at the focal SNP; each branch reflects the creation of new haplotypes, and the thickness of the line reflects the number of samples with the haplotype. EHH represents the probability that two randomly chosen chromosomes are identical by descent from the focal SNP to the current position of interest⁹⁰. Selection at the core allele is expected to result in EHH values close to 1 in an extended region centered on the focal SNP. To measure the deviation, we used selscan⁹¹ to compute the integrated haplotype score (iHS)⁹², which is defined as the log of the ratio of the integrated EHH for the derived allele over the integrated EHH for the ancestral allele. These values are then normalized in frequency bins across the whole genome (25 bins were used). Note that selscan's definition of the iHS differs from earlier definitions where the ancestral allele was in the numerator of the ratio^{91,92}. In our case, a large positive iHS indicates that a derived allele has had its frequency increase due to selection. We computed an approximate two-sided P value under the assumption that after normalization the iHS is approximately distributed as a standard normal. We also used selscan to compute nSL (number of segregation sites by length) scores⁹³. The nSL is similar to the iHS, but instead of integrating over genetic distance, the nSL uses the number of segregating sites as a measure of ‘distance’. Thus the nSL is more robust to demographic assumptions than the iHS as it does not depend on a genetic map. As with the iHS, we normalized the nSL scores within 25 frequency bins across the whole genome, and computed approximate two-sided P values assuming a standard normal distribution. The selscan program was run using its assumed default values. As we are focused on testing whether there is positive selection at the missense variant, we did not adjust the P values for multiple testing.

### Chromatin Immunoprecipitation

Samples were prepared from 3T3-L1 cells stably overexpressing wild type or the p.Arg457Gln variant of human CREBRF with a carboxy-terminal Myc-His tag using a Pierce

Agarose ChIP Kit (Thermo Scientific, #26156). An anti-cMyc antibody (ThermoFisher MA1-980) was used for immunoprecipitation according to the instructions of the manufacturer. As targets, we selected orthologs of fruit fly genes that had been demonstrated to be up- or down-regulated with 6h rapamycin treatment in wildtype but not regulated in REPTOR mutant fruit fly larvae (Tiebe, M. et al. REPTOR and REPTOR-BP Regulate Organismal Metabolism and Transcription Downstream of TORC1. Dev Cell 33, 272-284 (2015)). CREBRF is the human ortholog of REPTOR. Immunoprecipitated chromatin was subjected to quantitative PCR analysis using SYBRgreen quantification and primer sets designed to amplify the most likely promoter or upstream regulatory sequences of target genes as indicated by evolutionary conservation and ENCODE data (Yue et al. 2014). A 5% aliquot of the chromatin immunoprecipitation samples were used as input controls to calculation % enrichment.

### Generation of a Transgenic Mouse

Generating transgenic mice involves five basic steps: purification of a transgenic construct, harvesting donor zygotes, microinjection of transgenic construct, implantation of microinjected zygotes into the pseudo-pregnant recipient mice, and genotyping and analysis of transgene expression in founder mice. Methods for the generation of transgenic mice are known in the art and described, for example, by Cho et al., Curr Protoc Cell Biol. 2009 March; CHAPTER: Unit-19.11, which is incorporated herein in its entirety.

An expression vector, such as an expression vector encoding CREBRF or an expression vector encoding a CREBRF variant (e.g., Arg457Gln), is generated using standard methods known in the art. Construction of transgenes can be accomplished using any suitable genetic engineering technique, such as those described in Ausubel et al. (Current Protocols in Molecular Biology, John Wiley & Sons, New York, 2000). In one embodiment, the transgene is generated using CRISPR/Cas9 technology. Many techniques of transgene construction and of expression constructs for transfection or transformation in general are known and may be used to generate the desired CREBRF expressing construct.

One skilled in the art will appreciate that a promoter is chosen that directs expression of the CREBRF gene in all tissues or in a preferred tissue. In particular embodiments, CREBRF expression is driven by a phosphoglycerate kinase 1 promoter (PGK1)(Qin et al. (2010) PLoS ONE 5(5): e10611. doi:10.1371/journal.pone.0010611), the spleen focus-forming virus (SFFV) (Gonzalez-Murillo et al., Hum. Gene Ther. 2010 May; 21(5):623-30, using knockin technology (Cohen-Tannoudji et al., Mol Hum Reprod 4:929-938, 1998; Rossant et al., Nat Med 1:592-594, 1995; tet-off promoter (Clontech), human EFIs, CMV or endogenous CRBN promotor. The modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. Preferably, an intact region that includes all of the transcriptional regulatory elements of a gene is used.

Following its construction, the transgene construct is amplified by transforming bacterial cells using standard techniques. Plasmid DNA is then purified and treated to remove endogenous bacterial sequences. A fragment suitable for expression of a transgenic CREBRF under the control of a suitable promoter, such as an endogenous murine CREBRF promoter, and optionally additional regulatory elements is purified (e.g., by a sucrose gradient or a gel-purification method) in preparation for microinjection.

Foreign DNA is transferred into a mouse zygote by microinjection into the pronucleus. A fragment of the transgene DNA isolated above is microinjected into the male pronuclei of fertilized mouse eggs derived from, for example, a C57BL/6 or C3B6 Fl strain, using the techniques described in Gordon et al. (Proc. Natl. Acad. Sci. USA 77:7380, 1980). The eggs are transplanted into pseudopregnant female mice for full-term gestation, and resultant litters are analysed to identify transgenic mice.

In other embodiments, the knock-in of a mutant allele in the mouse genome can be achieved using homologous recombination (HR) in embryonic stem (ES) cells (Thomas and Capecchi 1987), similar to the methods used to generate conditional knockout mice. Specific mutations can be introduced into endogenous genes and transmitted throughout the mouse germ-line. A DNA construct containing the engineered gene of interest (e.g., a mutated oncogene) is flanked by sequences identical to those in the target locus and introduced into ES cells, where homologous sequences align and recombine, thereby introducing the altered gene into an endogenous locus. This technology allows for the expression of mutant genes from their endogenous promoter, or another promoter of interest, and avoids issues of variability and founder effects that are frequently observed with randomly integrated transgenes.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1. A Thrifty Variant in CREBRF Strongly Influences Body Mass Index (BMI)

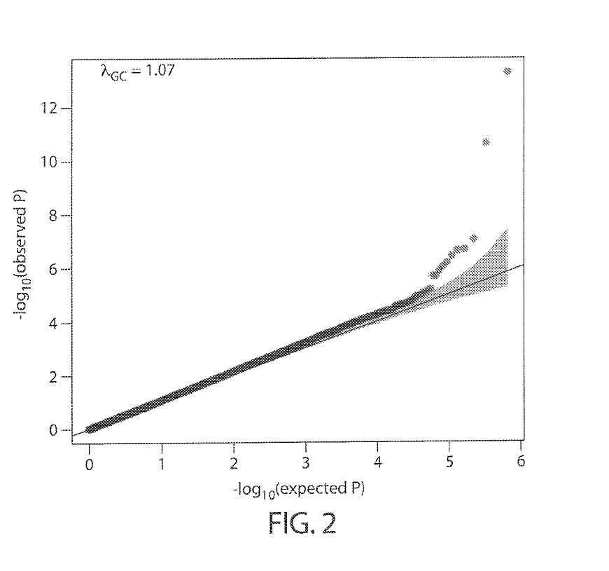

To discover genes influencing BMI, 659,492 markers genome-wide were genotyped in a discovery sample of 3,072 Samoans sampled recruited from 33 villages across the ‘Upolu and Savai’i islands using the Affymetrix 6.0 chip (Table 1, FIGS. 1A and 1B). Population substructure and inferred relatedness were adjusted for using an empirical kinship matrix; and association was tested using linear mixed models. Quantile-quantile (QQ) plots indicated that inflation was well-controlled (λ_{GC}=1.07) (FIG. 2).

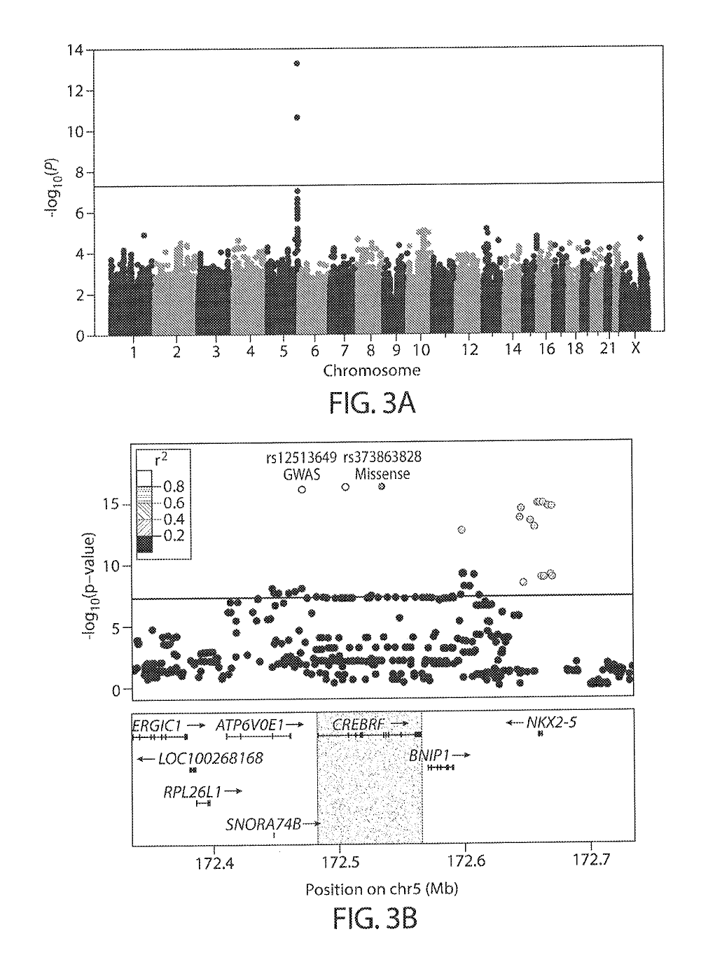

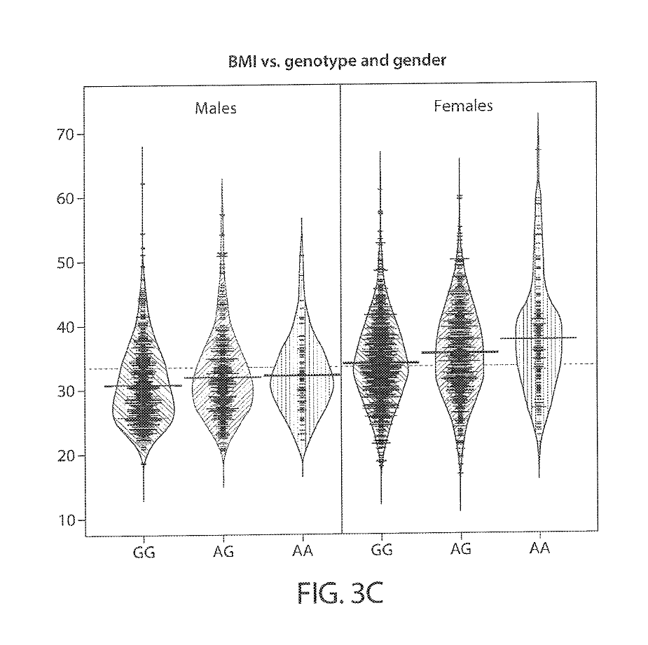

The strongest association with BMI occurred at rs12513649 (P=5.3×10′¹⁴) on chromosome 5q35.1 (FIG. 3A). This association was strongly replicated (P=1.2×10⁻⁹) in 2,102 adult Samoans from a 1990-95 longitudinal study and a 2002-03 family study, each drawn from both American Samoa and Samoa (Table 1, Table 2). While the BMI-increasing allele of rs12513649 is observed to be rare in people of African or European ancestry, it had a frequency of 0.258 in the samples. To fine-map the signal, Affymetrix-based genotypes were used to optimally select 96 individuals for targeted sequencing of a 1.5 Mb region centered on rs12513649. Haplotypes generated using the sequencing data were used to impute genotypes for the rest of the discovery sample. Analyses of the imputed data highlighted two significant single nucleotide polymorphisms (SNPs) in CREBRF, rs150207780 and rs373863828 (FIG. 3B). Due to high linkage disequilibrium in the area, conditional analyses were not able to distinguish between the top variants on statistical grounds (FIG. 4). Annotation indicated rs150207780 was intronic with no predicted regulatory function, and drew the attention to rs373863828, which was the only strongly associated missense variant among the 775 variants with P<1×10-5 in the targeted sequencing region. The rs373863828 missense variant (c.1370G>A, p.Arg457Gln) is located at a highly conserved position (GERP score 5.49) with a high probability of being damaging (SIFT: 0.03, PolyPhen2: 0.996). Furthermore, the BMI-increasing allele of rs373863828 (A) has an overall frequency of 0.259 in Samoans but is unobserved or extremely rare in other populations, with an allele count in the Exome Aggregation Consortium of only 5/121,362 (Table 2)¹². Bayesian finemapping with PAINTOR13 strongly supported following up the missense variant: the two variants in the region with the highest posterior probabilities (PPs) of being causal were rs373863828 (PP=0.80) and rs150207780 (PP=0.22); when ENCODE functional annotation was included, these probabilities increased to 0.92 and 0.34, respectively.

TABLE 1
Characteristics of genotyped individuals from the Smoan Studies

| | 2010 GWA Study | | | | 1991 Study | | | | 1990 Study | | | |
|---|---|---|---|---|---|---|---|---|---|---|---|---|
| | (Samoa) | | | | (Samoa) | | | | (American Samoa) | | | |
| | Men | | Women | | Men | | Women | | Men | | Women | |
| | (n = 1235) | | (n = 1837) | | (n = 291) | | (n = 316) | | (n = 188) | | (n = 225) | |
| Age (years) | 45.4 | (11.4) | 44.7 | (11.1) | 38.6 | (9.1) | 39.1 | (9.1) | 40.4 | (9.9) | 39.2 | (10.5) |
| Adiposity traits | | | | | | | | | | | | |
| BMI (kg/m²) | 31.3 | (5.9) | 34.9 | (6.8) | 28.9 | (4.9) | 30.9 | (5.3) | 33.9 | (5.9) | 36.0 | (7.0) |
| Body fat (%) | 24.0 | (11.8) | 37.2 | (11.8) | — | — | — | — | — | — | — | — |
| Abdominal circ. (cm) | 102.1 | (15.0) | 108.3 | (14.5) | 93.1 | (12.9) | 97.8 | (13.7) | 106.8 | (14.6) | 109.6 | (15.3) |
| Hip circ. (cm) | 105.7 | (10.2) | 114.5 | (12.6) | 100.0 | (9.0) | 107.3 | (10.5) | 111.0 | (11.8) | 118.2 | (13.8) |
| Abdominal-hip ratio | 0.962 | (0.07) | 0.945 | (0.07) | 0.928 | (0.07) | 0.909 | (0.07) | 0.961 | (0.06) | 0.928 | (0.08) |
| Obesity (>32 kg/m²) | 509 | 41% | 1195 | 65% | 65 | 22% | 129 | 41% | 114 | 61% | 162 | 72% |
| Metabolic traits | | | | | | | | | | | | |
| Fasting glucose | 89.6 | (14.4) | 88.0 | (13.6) | 85.3 | (12.3) | 84.7 | (11.0) | 94.4 | (10.8) | 93.1 | (10.7) |
| (mg/dL)^{†} | | | | | | | | | | | | |
| Fasting insulin | 12.5 | (13.7) | 16.2 | (14.4) | 10.4 | (11.3) | 12.2 | (10.8) | 19.8 | (24.9) | 21.4 | (17.2) |
| (μU/mL)^{†} | | | | | | | | | | | | |
| HOMA-IR^{†} | 2.9 | (3.6) | 3.6 | (3.6) | 2.3 | (3.0) | 2.6 | (2.6) | 4.9 | (7.4) | 5.1 | (4.5) |
| Adiponectin (μg/mL) | 4.9 | (2.5) | 6.1 | (3.1) | — | — | — | — | — | — | — | — |
| Leptin (ng/mL) | 7.7 | (7.0) | 25.5 | (13.8) | 4.3 | (4.4) | 17.0 | (9.2) | 10.1 | (22.6) | 25.7 | (11.7) |
| Diabetes | 185 | 16% | 293 | 17% | 9 | 3% | 12 | 3% | 25 | 13% | 17 | 8% |
| Hypertension | 441 | 36% | 583 | 32% | 60 | 21% | 41 | 13% | 57 | 30% | 53 | 24% |
| Serum lipid levels | | | | | | | | | | | | |
| Total cholesterol | 200.3 | (38.7) | 199.2 | (36.1) | 204.4 | (37.2) | 209.6 | (35.1) | 202.1 | (39.4) | 196.0 | (36.8) |
| (mg/dL) | | | | | | | | | | | | |
| Triglycerides (mg/dL) | 139.4 | (112.9) | 115.2 | (80.6) | 91.5 | (52.7) | 81.2 | (38.4) | 162.8 | (117.4) | 103.6 | (48.1) |
| HDL (mg/dL) | 43.7 | (11.2) | 46.5 | (10.8) | 40.5 | (11.6) | 43.3 | (10.4) | 36.0 | (7.6) | 38.3 | (8.1) |
| LDL (mg/dL) | 129.6 | (35.3) | 129.9 | (32.7) | 145.4 | (36.0) | 150.1 | (30.9) | 134.8 | (35.7) | 137.1 | (34.3) |

| | 2003 Study Adults | | | | 2002 Study Adults | | | | 2003 Study Children | | | |
|---|---|---|---|---|---|---|---|---|---|---|---|---|
| | (Samoa) | | | | (American Samoa) | | | | (Samoa) | | | |
| | Men | | Women | | Men | | Women | | Boys | | Girls | |
| | (n = 245) | | (n = 248) | | (n = 254) | | (n = 336) | | (n = 189) | | (n = 220) | |
| Age (years) | 40.9 | (16.3) | 44.0 | (17.0) | 43.0 | (16.5) | 43.0 | (16.0) | 11.3 | (3.5) | 11.6 | (3.5) |
| Adiposity traits | | | | | | | | | | | | |
| BMI (kg/m²) | 28.8 | (5.4) | 33.2 | (7.7) | 33.4 | (7.6) | 36.5 | (8.4) | 19.1 | (3.5) | 20.1 | (4.2) |
| Body fat (%) | 28.1 | (7.3) | 39.4 | (6.8) | 33.5 | (6.8) | 41.6 | (6.3) | 16.2 | (5.3) | 22.6 | (7.5) |
| Abdominal circ. (cm) | 95.5 | (14.9) | 107.0 | (16.5) | 107.5 | (16.4) | 111.0 | (16.5) | 67.0 | (9.7) | 70.4 | (12.3) |
| Hip circ. (cm) | 103.3 | (9.7) | 114.8 | (14.2) | 113.5 | (15.7) | 123.2 | (16.2) | 77.0 | (12.2) | 82.1 | (14.4) |
| Abdominal-hip ratio | 0.921 | (0.08) | 0.931 | (0.08) | 0.947 | (0.07) | 0.902 | (0.08) | 0.873 | (0.05) | 0.859 | (0.05) |
| Obesity (>32 kg/m²) | 59 | 24% | 130 | 52% | 138 | 54% | 229 | 68% | 5* | 3%* | 13* | 6%* |
| Metabolic traits | | | | | | | | | | | | |
| Fasting glucose | 88.6 | (11.4) | 89.8 | (12.2) | 88.1 | (14.9) | 86.8 | (15.8) | 83.4 | (8.4) | 82.4 | (3.4) |
| (mg/dL)^{†} | | | | | | | | | | | | |
| Fasting insulin | 7.1 | (9.1) | 10.0 | (9.6) | 12.7 | (13.4) | 14.5 | (17.9) | 5.1 | (5.0) | 8.6 | (10.3) |
| (μU/mL)^{†} | | | | | | | | | | | | |
| HOMA-iR^{†} | 1.7 | (2.4) | 2.4 | (2.6) | 2.9 | (3.3) | 3.3 | (4.7) | 1.1 | (1.1) | 1.8 | (2.5) |
| Adiponectin (μg/mL) | 10.0 | (8.4) | 12.5 | (7.9) | 8.1 | (5.5) | 11.0 | (9.7) | 13.9 | (10.7) | 13.7 | (6.3) |
| Leptin (ng/mL) | 6.4 | (6.9) | 24.5 | (14.1) | 11.4 | (9.7) | 30.0 | (15.8) | 4.0 | (3.9) | 9.8 | (7.7) |
| Diabetes | 19 | 8% | 25 | 10% | 58 | 23% | 65 | 19% | — | — | — | — |
| Hypertension | 68 | 28% | 75 | 30% | 119 | 47% | 117 | 35% | — | — | — | — |
| Serum lipid levels | | | | | | | | | | | | |
| Total cholesterol (mg/dL) | 195.8 | (40.4) | 202.3 | (35.9) | 189.5 | (37.9) | 187.2 | (38.6) | 158.9 | (25.0) | 168.3 | (26.9) |
| Triglycerides (mg/dL) | 120.3 | (91.4) | 110.9 | (58.9) | 200.2 | (207.3) | 130.9 | (78.3) | 73.4 | (27.6) | 87.1 | (44.6) |
| HDL (mg/dL) | 46.3 | (11.2) | 47.2 | (10.2) | 38.6 | (8.8) | 42.1 | (8.5) | 49.7 | (11.2) | 49.8 | (11.4) |
| LDL (mg/dL) | 126.1 | (37.7) | 133.0 | (32.2) | 118.2 | (34.8) | 118.9 | (34.3) | 94.8 | (21.0) | 101.5 | (24.7) |

Summary statistics based on those who were both phenotyped and successfully genotyped (for either rs12513649 or rs373863828). Numbers are means and (standard deviations) for all traits except obesity, diabetes and hypertension, which are counts and percentages. Percent body fat and serum adiponectin are not available for the 1990-91 Studies; self-reported diabetes and hypertension were exclusion criteria for the 1990-91 Studies.
^{†}Non-diabetics only (n = 966 men, n = 1,423 women).
*Children were classified as obese per Cole et al.³⁸

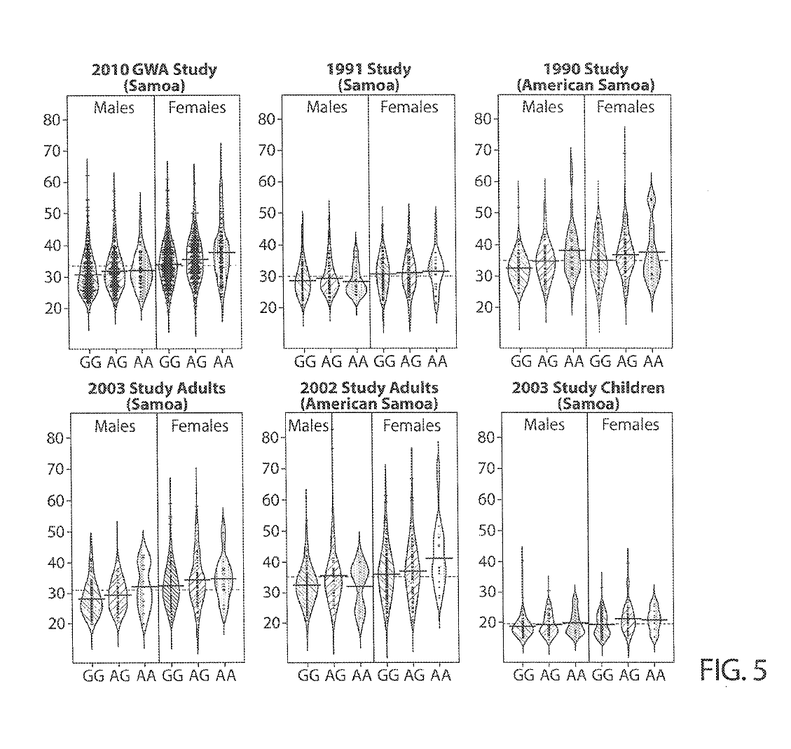

The missense variant rs373863828 was genotyped in the discovery and replication samples, obtaining very significant evidence of association with BMI in adults (P=7.0×10⁻¹³) (P=3.5×10⁻⁹), with a combined meta analysis P-value of 1.4×10⁻²° (Table 2, Table 3). The meta-analysis showed no evidence of heterogeneity among the three studies (I²=0%; Q=1.12; P=0.571). In the discovery sample, each copy of the A allele increased BMI by 1.36 kg/m² (FIG. 3C). In the replication sample, each copy of the A allele increased BMI by 1.45 kg/m². In the discovery sample, each copy of the A allele increased BMI by 1.58 kg/m² in females and 0.83 kg/m² in males (FIG. 3C). Similarly, in the replication sample, the effect size was larger in women than in men in certain sub-groups (Table 2). There was a strong effect on BMI at this locus even after stratifying by sex and cohort (FIG. 5; however, sex x genotype interactions were not significant [Discovery P=0.060; Replication P=0.555]). There was also suggestive evidence (P=1.1×10⁻³) that this variant increased BMI in the small sample of 409 Samoan children (Table 2). The rs373863828 p.Arg457Gln variant accounted for 1.93% of the variance in BMI in the discovery sample and 1.08% in the replication sample. In comparison, rs1558902, the main risk variant in FTO, increases BMI by 0.39 kg/m² and accounts for only 0.34% of the BMI variance in Europeans^{14,15}. Based on searching the literature and databases (including GRASP^{16,17}), there were no any significant associations with BMI in the CREBRF region in any other human studies.

TABLE 2
Association details for rs12513649 and rs373863828

| | Discovery | Missense |
|---|---|---|
| Attribute | variant | variant |
| SNP RS ID | rs12513649 | rs373863828 |
| Chromosome | 5 | 5 |
| Physical position (GRCh37.p13) | 172472052 | 172535774 |
| Effect allele | G | A |
| Other allele | C | G |
| Nearest gene upstream of the SNP | ATP6V0E1 | CREBRF |
| Distance in base pairs to nearest upstream gene | 10152 | 0.000 |
| Nearest gene downstream of the SNP | CREBRF | CREBRF |
| Distance in base pairs to nearest downstream gene | 11302 | 0.000 |
| P values | | |
| GWAS Samoans from the 2010s | 5.3e−14 | 7.0e−13 |
| Samoans from 1990s | 5.8e−04 | 8.0e−04 |
| Samoan adults from the 2000s | 3.0e−07 | 6.5e−07 |
| Samoan children from the 2000s | 4.1e−03 | 1.1e−03 |
| Meta-analysis of the 1990s and 2000s samples | 1.2e−09 | 3.5e−09 |
| Meta-analysis of the 1990s, 2000s and 2010s samples | 4.0e−22 | 1.4e−20 |
| Effect sizes (betas) for log-transformed BMI | | |
| GWAS Samoans from the 2010s | 0.041(0.005) | 0.039(0.005) |
| Samoans from the 1990s | 0.029(0.008) | 0.028(0.008) |
| Samoans adults from the 2000s | 0.056(0.011) | 0.054(0.011) |
| Samoan children from the 2000s | 0.031(0.011) | 0.035(0.011) |
| Direction of the effect in each of the four samples | ++++ | ++++ |
| Sample sizes (phenotyped and genotyped) | | |
| GWAS Samoans from the 2010s (Discovery) | 3072 | 3066 |
| Samoans from 1990 (Replication)s | 1020 | 1020 |
| Samoan adults from the 2000s (Replication) | 1082 | 1083 |
| Samoan children from the 2000s | 409 | 409 |
| Meta-analysis of the 1990s and 2000s samples | 2102 | 2103 |
| Meta-analysis of the 1990s, 2000s and 2010s samples. | 5174 | 5169 |
| Effect allele frequencies | | |
| GWAS Samoans from the 2010s | 0.276 | 0.276 |
| Samoans from 1990s | 0.251 | 0.251 |
| Samoan adults from the 2000s | 0.224 | 0.225 |
| Samoan children from the 2000s | 0.236 | 0.235 |
| All of the 1990s, 2000s and 2010s samples | 0.258 | 0.259 |
| Individuals of East Asian descent from 1000G | 0.063 | 0.000 |
| Individuals of South Asian descent from 1000G | 0.003 | 0.000 |
| Individuals of European descent from 1000G | 0.000 | 0.000 |
| Individuals of admixed American descent from 1000G | 0.059 | 0.000 |
| Individuals of African descent from 1000G | 0.001 | 0.000 |
| Individuals of East Asian descent from ExAC | N/A | <0.001* |
| Individuals of South Asian descent from ExAC | N/A | 0.000 |
| Individuals of European descent from ExAC | N/A | <0.001^{†} |
| Individuals of Latino American descent from ExAC | N/A | 0.000 |
| Individuals of African descent from ExAC | N/A | 0.000 |
| Individuals of other descent from ExAC | N/A | 0.001^{‡} |

This table provides detailed results for rs12513649 and rs373863828.
Abbreviation:
1000G, 1000 Genomes Project;
ExAC, Exon Aggregation Consortium¹²;
N/A, not available
*2A alleles in 8,636 measured alleles.
^{†}2A alleles in 73,328 measured alleles.
^{‡}1A allele in 908 measured alleles.

TABLE 3
Demographics and association of rs373863828 with BMI

| 2010 GWA Study | | 1991 Study | | 1990 Study | |
|---|---|---|---|---|---|
| (Samoa) | | (Samoa) | | (American Samoa) | |
| Women | Men | Women | Men | Women | Men |
| n = 1837 | n = 1235 | n = 318 | n = 296 | n = 227 | n = 189 |
| mean (s.d.) | mean (s.d.) | mean (s.d.) | mean (s.d.) | mean (s.d.) | mean (s.d.) |

| | | | | | | | | | | | | |
|---|---|---|---|---|---|---|---|---|---|---|---|---|
| age (years) | 44.7 | (11.1) | 45.4 | (11.4) | 39.1 | (9.0) | 38.5 | (9.1) | 39.2 | (10.5) | 40.5 | (9.9) |
| BMI (kg/m²) | 34.9 | (6.8) | 31.3 | (5.9) | 30.9 | (5.3) | 28.9 | (4.9) | 36.0 | (6.9) | 33.9 | (5.9) |

BMI (kg/m²) stratified by rs373863828*

| | | | | | | | | | | | | |
|---|---|---|---|---|---|---|---|---|---|---|---|---|
| GG (55.3%) | 34.0 | (6.4) | 30.7 | (5.8) | 30.7 | (5.1) | 28.7 | (4.8) | 35.2 | (6.4) | 32.6 | (4.7) |
| GA (37.6%) | 35.5 | (6.7) | 31.9 | (6.1) | 31.2 | (5.6) | 29.5 | (5.0) | 37.0 | (7.3) | 34.8 | (5.9) |
| AA (7.1%) | 37.6 | (8.4) | 32.1 | (5.8) | 31.6 | (6.0) | 28.3 | (4.4) | 37.6 | (9.6) | 38.2 | (8.4) |

| | 2003 Study Adults | | | | 2002 Study Adults | | | | 2003 Study Children | | | |
|---|---|---|---|---|---|---|---|---|---|---|---|---|
| | (Samoa) | | | | (American Samoa) | | | | (Samoa) | | | |
| | Women | | Men | | Women | | Men | | Girls | | Boys | |
| | n = 248 | | n = 245 | | n = 337 | | n = 254 | | n = 220 | | n = 191 | |
| | mean (s.d.) | | mean (s.d.) | | mean (s.d.) | | mean (s.d.) | | mean (s.d.) | | mean (s.d.) | |
| age (years) | 44.0 | (17.0) | 40.9 | (16.3) | 43.0 | (16.0) | 43.0 | (16.5) | 11.6 | (3.5) | 11.3 | (3.5) |
| BMI (kg/m²) | 33.2 | (7.7) | 28.8 | (5.4) | 36.5 | (8.4) | 33.4 | (7.6) | 20.1 | (4.2) | 19.1 | (3.5) |

BMI (kg/m²) stratified by rs373863828*

| | | | | | | | | | | | | |
|---|---|---|---|---|---|---|---|---|---|---|---|---|
| GG (55.3%) | 32.4 | (7.4) | 28.0 | (5.2) | 35.9 | (7.9) | 32.4 | (5.7) | 19.4 | (3.7) | 18.8 | (3.4) |
| GA (37.6%) | 34.3 | (8.1) | 29.4 | (5.2) | 37.0 | (8.9) | 35.5 | (10.3) | 21.4 | (4.9) | 19.4 | (3.6) |
| AA (7.1%) | 34.4 | (7.0) | 32.0 | (7.5) | 41.3 | (10.5) | 31.9 | (7.2) | 21.0 | (4.0) | 19.9 | (3.9) |

*Genotype frequencies are those of all samples combined. n_{GG} = 3087, n_{GA} = 2097, n_{AA} = 394

In addition to BMI, the A allele was also positively associated with obesity risk (OR 1.305 and 1.441 in discovery and replication cohorts, respectively) as well as measures of total and regional adiposity including percent body fat, abdominal circumference, and hip circumference in both cohorts (Table 4 and Table 5). The A allele was also positively associated with serum leptin in women (both cohorts) and men (replication cohort) before but not after adjusting for BMI. These data indicate that the association between the missense variant and BMI is indeed due to an association with adiposity.

Given the strength of the association of rs373863828 with BMI, associations between this SNP and fifteen adiposity, metabolic, and lipid health outcome phenotypes were examined (Table 4). The BMI-increasing allele (A) was positively associated with abdominal circumference, hip circumference, percent body fat, abdominal—hip ratio, hypertension risk, and obesity risk and negatively associated with total cholesterol, fasting glucose, and diabetes risk at a Bonferroni-corrected significance threshold of p=0.0027. Fasting insulin and leptin levels were positively associated with the BMI-increasing allele in models that do not include BMI as a covariate, but not in models that include it, indicative of an effect of the allele on these traits through its influence on BMI.

TABLE 4
Association of rs373863828 with adiposity, metabolic, and lipid traits.

| Quantitative Trait | n | β | s.e. | P | Covariates* |
|---|---|---|---|---|---|
| Adiposity traits | | | | | |
| Body fat (%) | 2893 | 2.199 | 0.345 | 1.78e−10 | A, A², S, A × S |
| Abdominal circ. | 3057 | 2.842 | 0.404 | 2.05e−12 | A, A², S, A × S, A² × S |
| Hip circ. | 3058 | 2.361 | 0.332 | 1.19e−12 | A, A², S, A² × S |
| Abdominal-hip ratio | 3056 | 0.005 | 0.002 | 2.23e−03 | A, A², S, A × S, A² × S |
| Metabolic traits | | | | | |
| Fasting glucose† | 2393 | −1.652 | 0.423 | 9.52e−05 | A, A², S |
| Fasting insulin† | 2392 | 1.342 | 0.449 | 2.83e−03 | A, S, A × S |
| HOMA-IR† | 2392 | 0.241 | 0.114 | 0.035 | A, S, A × S |
| Adiponectin | 2858 | −0.228 | 0.083 | 6.30e−03 | A, A², S, A × S |
| Leptin (men)‡ | 1151 | 0.719 | 0,326 | 0.027 | A |
| Leptin (women)‡ | 1707 | 1.888 | 0.525 | 3.25e−04 | |
| Metabolic traits | | | | | |
| adjusted for BMI | | | | | |
| Fasting glucose† | 2383 | −2.248 | 0.417 | 6.89e−08 | A, A², S, B |
| Fasting insulin† | 2382 | 0.225 | 0.420 | 0.592 | A, A², S, B, A × S, A² × S |
| HOMA-IR† | 2382 | −0.034 | 0.107 | 0.754 | A, B |
| Adiponectin | 2844 | −0.066 | 0.080 | 0.412 | A, A², S, B, A × S |
| Leptin (men)‡ | 1143 | −0.262 | 0.210 | 0.213 | A, A², B |
| Leptin (women)‡ | 1701 | −0.516 | 0.366 | 0.159 | A, A², B |
| Serum lipid levels | | | | | |
| Total cholesterol | 2858 | −3.203 | 1.029 | 0.002 | A, A², S, A × S, A² × S |
| Triglycerides | 2858 | 0.349 | 2.769 | 0.900 | A, S, A × S |
| HDL | 2858 | −0.322 | 0.321 | 0.317 | A, A², S |
| LDL | 2851 | −2.347 | 0.945 | 0.013 | A, A², S, A² × S |

| Dichotomous Trait | n | OR | 95% CI | p | Covariates* |
|---|---|---|---|---|---|
| Obesity | 3066 | 1.305 | (1.159, 1.470) | 1.12e−05 | A, A², S, A × S |
| Diabetes | 2876 | 0.637 | (0.536, 0.758) | 3.86e−07 | A |
| Diabetes adjusted for BMI | 2861 | 0.586 | (0.489, 0.702) | 6.68e−09 | A, B |
| Hypertension | 3041 | 1.014 | (0.898, 1.145) | 0.818 | A, S |

Boldface represents a P value < 0.0027.
*A = age, A² = age², S = sex, A × S = age × sex interaction, A² × S = age² × sex interaction, B = log(BMI)
†Analysis conducted only in non-diabetics
‡Leptin was not analysed in men and women combined because the distributions in each sex were very different.
Abbreviations:
s.e., standard error;
OR, odds ratio;
95% CI, 95% confidence interval

TABLE 5
Association of rs373863828 with untransformed adiposity, metabolic, and lipid
traits in (a) the discovery sample and (b) the adult replication sample.

| (a) Discovery sample | All adults | | | | | Men | | |
|---|---|---|---|---|---|---|---|---|
| Quantitative Trait | n | β | s.e. | P | Covariates* | n | β | s.e. |
| Adiposity traits | | | | | | | | |
| BMI (kg/m²) | 3066 | 1.356 | 0.183 | 1.12E−13 | A, A², S, | 1233 | 0.967 | 0.265 |
| | | | | | A × S | | | |
| Body fat (%) | 2893 | 2.199 | 0.345 | 1.78E−10 | A, A², S, | 1150 | 1.677 | 0.546 |
| | | | | | A × S | | | |
| Abdominal circ. (cm) | 3057 | 2.842 | 0.404 | 2.05E−12 | A, A², S, | 1231 | 2.258 | 0.638 |
| | | | | | A × S, A² × S | | | |
| Hip circ. (cm) | 3058 | 2.361 | 0.332 | 1.19E−12 | A, A², S, | 1230 | 1.769 | 0.462 |
| | | | | | A² × S | | | |
| Abdominal-hip ratio | 3056 | 0.005 | 0.002 | 2.23E−03 | A, A², S, | 1230 | 0.005 | 0.003 |
| | | | | | A × S, A² × S | | | |
| Metabolic traits | | | | | | | | |
| Fasting glucose | 2393 | −1.652 | 0.423 | 9.52E−05 | A, A², S | 970 | −2.448 | 0.687 |
| (mg/dL)^{†} | | | | | | | | |
| Fasting insulin | 2392 | 1.342 | 0.449 | 0.003 | A, S, A × S | 970 | 0.619 | 0.684 |
| (μU/mL)^{†} | | | | | | | | |
| HOMA-IR^{†} | 2392 | 0.241 | 0.114 | 0.035 | A, S, A × S | 970 | 0.080 | 0.181 |
| Adiponectin (μg/mL) | 2858 | −0.228 | 0.083 | 0.006 | A, A², S, A × S | 1151 | −0.251 | 0.113 |
| Leptin (ng/mL)^{‡} | — | — | — | — | | 1151 | 0.719 | 0.326 |
| Metabolic traits | | | | | | | | |
| adjusted for BMI | | | | | | | | |
| Fasting glucose | 2383 | −2.248 | 0.417 | 6.89E−08 | A, A², S, B | 964 | −2.833 | 0.682 |
| (mg/dL)^{†} | | | | | | | | |
| Fasting insulin | 2382 | 0.225 | 0.420 | 0.592 | A, A², S, B, | 964 | −0.224 | 0.632 |
| (μU/mL)^{†} | | | | | A × S, A² × S | | | |
| HOMA-IR^{†} | 2382 | −0.034 | 0.107 | 0.754 | A, B | 964 | −0.130 | 0.170 |
| Adiponectin (μg/mL) | 2844 | −0.066 | 0.080 | 0.412 | A, A², S, B, | 1143 | −0.130 | 0.109 |
| | | | | | A × S | | | |
| Leptin (ng/mL)^{‡} | — | — | — | — | | 1143 | −0.262 | 0.210 |
| Serum lipid levels | | | | | | | | |
| Total cholesterol | 2858 | −3.203 | 1.029 | 1.84E−03 | A, A², S, | 1151 | −3.423 | 1.731 |
| (mg/dL) | | | | | A × S, A² × S | | | |
| Triglycerides (mg/dL) | 2358 | 0.349 | 2.769 | 0.900 | A, S, A × S | 1151 | −5.838 | 5.220 |
| HDL (mg/dL) | 2858 | −0.322 | 0.321 | 0.317 | A, A², S | 1151 | 0.406 | 0.516 |
| LDL (mg/dL) | 2851 | −2.347 | 0.945 | 0.013 | A, A², S, | 1145 | −2.115 | 1.586 |
| | | | | | A² × S | | | |

| (a) Discovery sample | Men | | Women | | | | |
|---|---|---|---|---|---|---|---|
| Quantitative Trait | P | Covariates* | n | β | s.e. | P | Covariates* |
| Adipocity traits | | | | | | | |
| BMI (kg/m²) | 2.57E−04 | A, A² | 1833 | 1.644 | 0.247 | 2.75E−11 | A, A² |
| Body fat (%) | 2.20E−03 | A, A² | 1743 | 2.559 | 0.442 | 6.92E−09 | A, A² |
| Abdominal circ. (cm) | 3.98E−04 | A, A² | 1826 | 3.235 | 0.520 | 5.01E−10 | A, A² |
| Hip circ. (cm) | 1.30E−04 | A, A² | 1328 | 2.776 | 0.458 | 1.31E−09 | A, A² |
| Abdominal-hip ratio | 0.051 | A, A² | 1826 | 0.005 | 0.002 | 0.019 | A |
| Metabolic traits | | | | | | | |
| Fasting glucose | 3.62E−04 | A, A² | 1423 | −1.019 | 0.535 | 0.057 | A, A² |
| (mg/dL)^{†} | | | | | | | |
| Fasting insulin | 0.365 | | 1422 | 1.809 | 0.592 | 2.23E−03 | A |
| (μU/mL)^{†} | | | | | | | |
| HOMA-IR? | 0.660 | A, A² | 1422 | 0.355 | 0.146 | 0.015 | A |
| Adiponectin (μg/mL) | 0.027 | A, A² | 1707 | −0.235 | 0.116 | 0.043 | A, A² |
| Leptin (ng/mL)^{‡} | 0.027 | A | 1707 | 1.888 | 0.525 | 3.25E−04 | |
| Metabolic traits | | | | | | | |
| adjusted for BMI | | | | | | | |
| Fasting glucose | 3.24E−05 | A, A², B | 1419 | −1.756 | 0.524 | 8.01E−04 | A, B |
| (mg/dL)^{†} | | | | | | | |
| Fasting insulin | 0.723 | B | 1418 | 0.513 | 0.557 | 0.357 | A, A², B |
| (μU/mL)^{†} | | | | | | | |
| HOMA-IR^{†} | 0.444 | B | 1418 | 0.029 | 0.138 | 0.834 | A, B |
| Adiponectin (μg/mL) | 0.233 | A, A², B | 1701 | −0.042 | 0.111 | 0.707 | A, A², B |
| Leptin (ng/mL)^{‡} | 0.213 | A, A², B | 1701 | −0.516 | 0.366 | 0.159 | A, A², B |
| Serum lipid levels | | | | | | | |
| Total cholesterol | 0.048 | A, A² | 1707 | −3.319 | 1.256 | 0.008 | A, A² |
| (mg/dL) | | | | | | | |
| Triglycerides (mg/dL) | 0.263 | A | 1707 | 4.676 | 2.981 | 0.117 | A |
| HDL (mg/dL) | 0.431 | A | 1707 | −0.914 | 0.403 | 0.025 | A |
| LDL (mg/dL) | 0.182 | A, A² | 1706 | −2.647 | 1.155 | 0.022 | A, A² |

| Dichotomous Trait | n | OR | 95% CI | P | Covariates* | n | OR | 95% CI |
|---|---|---|---|---|---|---|---|---|
| Obesity | 3066 | 1.305 | (1.159, 1.470) | 1.12E−05 | A, A², | 1233 | 1.270 | (1.052, 1.535) |
| (>32 kg/m²) | | | | | S, A × S | | | |
| Diabetes | 2876 | 0.637 | (0.536, 0.756) | 3.86E−07 | A | 1157 | 0.611 | (0.461, 0.811) |
| Diabetes adj. | 2861 | 0.586 | (0.489, 0.702) | 6.68E−09 | A, B | 1149 | 0.623 | (0.495, 0.784) |
| for BMI | | | | | | | | |
| Hypertension | 3041 | 1.014 | (0.898, 1.145) | 0.818 | A, S | 1226 | 0.923 | (0.760, 1.120) |

| Dichotomous Trait | P | Covariates* | n | OR | 95% CI | P | Covariates* |
|---|---|---|---|---|---|---|---|
| Obesity | 0.013 | A, A² | 1833 | 1.335 | (1.144, 1.557) | 2.38E−04 | A, A² |
| (>32 kg/m²) | | | | | | | |
| Diabetes | 6.31E−04 | A, A² | 1719 | 0.669 | (0.537, 0.833) | 3.40E−04 | A, A² |
| Diabetes adj. | 5.49E−05 | A, A², | 1712 | 0.566 | (0.422, 0.760) | 1.50E−04 | A, A², B |
| for BMI | | B | | | | | |
| Hypertension | 0.416 | A | 1815 | 1.087 | (0.930, 1.269) | 0.295 | A |

Boldface represents a P value < 2.17E−03.
*A = age, A² = age², S = sex, A × S = age × sex interaction,
A² × S = age² × sex interaction, B = log(BMI).
^{†}Analysis conducted only in non-diabetics
^{‡}Leptin was not analyzed in men and women combined because
the distributions in each sex were very different.
Abbreviations:
s.e., standard error;
OR, odds ratio;
95% CI, 95% confidence interval;
circ., circumference;
adj., adjusted

| (b) Replication sample | | | | | | | | |
|---|---|---|---|---|---|---|---|---|
| (mega analysis) | All adults | | | | | Men | | |
| Quantitative Trait | n | β | s.e. | P | Covariates* | n | β | s.e. |
| Adiposity traits | | | | | | | | |
| BMI (kg/m²) | 2103 | 1.453 | 0.237 | 8.22E−10 | A, A², S, N, C | 978 | 1.501 | 0.306 |
| Body fat (%) | 880 | 1.335 | 0.392 | 6.58E−04 | A, A², S, | 401 | 1.192 | 0.595 |
| | | | | | A × S, N | | | |
| Abdominal circ. (cm) | 2172 | 3.218 | 0.518 | 5.12E−10 | A, A², S, N, C | 1003 | 3.318 | 0.704 |
| Hip circ. (cm) | 2165 | 2.716 | 0.462 | 4.27E−09 | A, A², S, N, C | 1002 | 2.838 | 0.605 |
| Abdominal-hip ratio | 2162 | 0.006 | 0.002 | 0.017 | A, A², S, | 1001 | 0.006 | 0.003 |
| | | | | | A × S, A² × S, N, C | | | |
| Metabolic traits | | | | | | | | |
| Fasting glucose | 1948 | −1.541 | 0.463 | 8.84E−04 | A, A², S, N | 901 | −1.508 | 0.669 |
| (mg/dL)^{†} | | | | | | | | |
| Fasting insulin | 1947 | 2.500 | 0.565 | 9.55E−06 | A, A², S, | 900 | 2.595 | 0.838 |
| (μU/mL)^{†} | | | | | A × S, N, C | | | |
| HOMA-IR^{†} | 1947 | 0.572 | 0.150 | 1.43E−04 | A, A², S, | 900 | 0.663 | 0.228 |
| | | | | | A × S, N, C | | | |
| Adiponectin (μg/mL) | 1079 | −1.078 | 0.426 | 0.011 | A, A², S, | 497 | −1.153 | 0.529 |
| | | | | | A × S, A² × S, N | | | |
| Leptin (ng/mL)^{‡} | — | — | — | — | | 831 | 2.237 | 0.607 |
| Metabolic traits | | | | | | | | |
| adjusted for BMI | | | | | | | | |
| Fasting glucose | 1867 | −2.094 | 0.468 | 7.62E−06 | A, A², S, B, N | 866 | −2.137 | 0.672 |
| (mg/dL)^{†} | | | | | | | | |
| Fasting insulin | 1866 | 1.557 | 0.539 | 0.004 | A, A², S, B, | 865 | 1.874 | 0.781 |
| (μU/mL)^{†} | | | | | A × S, N, C | | | |
| HOMA-IR^{†} | 1866 | 0.358 | 0.147 | 0.015 | A, S, B, | 865 | 0.475 | 0.220 |
| | | | | | A × S, N, C | | | |
| Adiponectin (μg/mL) | 1068 | −0.780 | 0.428 | 0.068 | A, A², S, B, | 491 | −0.928 | 0.527 |
| | | | | | A × S, A² × S, N | | | |
| Leptin (ng/mL)^{‡} | — | — | — | — | | 801 | 0.863 | 0.521 |
| Serum lipid levels | | | | | | | | |
| Total cholesterol | 1849 | −1.808 | 1.344 | 0.157 | A, A², S, | 860 | −0.891 | 1.945 |
| (mg/dL) | | | | | A × S, A² × S, N | | | |
| Triglycerides (mg/dL) | 1849 | −4.888 | 4.153 | 0.239 | A, A², S, | 660 | −13.11 | 7.729 |
| | | | | | A × S, A² × S, N, C | | | |
| HDL (mg/dL) | 1834 | −1.097 | 0.391 | 0.005 | A, A², S, N, C | 848 | −1.088 | 0.578 |
| LDL (mg/dL) | 1805 | −1.047 | 1.291 | 0.417 | A, A², S, | 825 | 0.156 | 1.951 |
| | | | | | A × S, A² × S, N, C | | | |

| (b) Replication | | | | | | | |
|---|---|---|---|---|---|---|---|
| sample (mega | | | | | | | |
| analysis) | Men | | Women | | | | |
| Quantitative Trait | P | Covariates* | n | β | s.e. | P | Covariates* |
| Adiposity traits | | | | | | | |
| BMI (kg/m²) | 9.54E−07 | A, A², N | 1125 | 1.389 | 0.348 | 6.41E−05 | A, A², N, C |
| Body fat (%) | 0.045 | A, A², N | 479 | 1.314 | 0.491 | 0.007 | A, A², N |
| Abdominal circ. (cm) | 2.42E−06 | A, A², N, C | 1164 | 3.087 | 0.735 | 2.64E−05 | A, A², N, C |
| Hip circ. (cm) | 2.76E−06 | A, A², N, C | 1163 | 2.597 | 0.674 | 1.16E−04 | A, A², N, C |
| Abdominal-hip ratio | 0.052 | A, A², N, C | 1161 | 0.005 | 0.004 | 0.126 | A, A² |
| Metabolic traits | | | | | | | |
| Fasting glucose | 0.024 | A, A², N | 1047 | −1.764 | 0.634 | 0.005 | A, A², N |
| (mg/dL)^{†} | | | | | | | |
| Fasting insulin | 1.96E−03 | N, C | 1047 | 2.174 | 0.740 | 0.003 | A², N, C |
| (μU/mL)^{†} | | | | | | | |
| HOMA-IR^{†} | 0.004 | A, A², N, C | 1047 | 0.440 | 0.191 | 0.022 | A, A², N, C |
| Adiponectin (μg/mL) | 0.029 | A, A², N | 582 | −0.878 | 0.628 | 0.162 | A, A², N |
| Leptin (ng/mL)^{‡} | 2.26E−04 | A, A², N, C | 952 | 2.548 | 0.726 | 4.47E−04 | A, A², N, C |
| Metabolic traits | | | | | | | |
| adjusted for BMI | | | | | | | |
| Fasting glucose | 1.47E−03 | A, A², B, N | 1001 | −2.274 | 0.642 | 3.96E−04 | A, A², B, N |
| (mg/dL)^{†} | | | | | | | |
| Fasting insulin | 0.016 | B, N, C | 1001 | 1.185 | 0.723 | 0.101 | A, A², B, N, C |
| (μU/mL)^{†} | | | | | | | |
| HOMA-IR^{†} | 0.031 | B, N, C | 1001 | 0.221 | 0.190 | 0.245 | A, B, N, C |
| Adiponectin (μg/mL) | 0.078 | A, A², B, N | 577 | −0.439 | 0.633 | 0.488 | A², B |
| Leptin (ng/mL)^{‡} | 0.098 | A, A², B, C | 919 | −0.009 | 0.511 | 0.935 | A, A², B, N, C |
| Serum lipid levels | | | | | | | |
| Total cholesterol | 0.647 | A, A², N | 989 | −2.525 | 1.812 | 0.163 | A, A², N, C |
| (mg/dL) | | | | | | | |
| Triglycerides (mg/dL) | 0.090 | A, A², N, C | 989 | 1.683 | 3.629 | 0.643 | A, A², N, C |
| HDL (mg/dL) | 0.060 | A, A², N, C | 986 | −0.948 | 0.516 | 0.066 | A, A², N, C |
| LDL (mg/dL) | 0.936 | A, A², N, C | 930 | −1.857 | 1.671 | 0.266 | A, A², N, C |

| Dichotomous | | | | | | | | |
|---|---|---|---|---|---|---|---|---|
| Trait | n | OR | 95% CI | P | Covariates* | n | OR | 95% CI |
| Obesity | 2103 | 1.441 | (1.227, 1.692) | 8.49E—06 | A, A², S, | 978 | 1.586 | (1.252, 2.009) |
| (>32 kg/m²) | | | | | N, C | | | |
| Diabetes | 2145 | 0.831 | (0.639, 1.081) | 0.168 | A, A², S, | 1000 | 0.698 | (0.472, 1.031) |
| | | | | | A × S, N, C | | | |
| Diabetes adj. | 2053 | 0.742 | (0.567, 0.969) | 0.029 | A, A², S, | 960 | 0.550 | (0.358, 0.845) |
| for BMI | | | | | B, N, C | | | |
| Hypertension | 2173 | 1.045 | (0.881, 1.240) | 0.613 | A, A², S, | 1006 | 1.029 | (0.817, 1.296) |
| | | | | | A × S, N, C | | | |

| Dichotomous | | | | | | | |
|---|---|---|---|---|---|---|---|
| Trait | P | Covariates* | n | OR | 95% CI | P | Covariates* |
| Obesity | 1.35E−04 | A, A², N | 1125 | 1.32 | (1.061, 1.643) | 0.013 | A, A², N, C |
| (>32 kg/m²) | | | | | | | |
| Diabetes | 0.071 | A, A², N, C | 1145 | 0.950 | (0.670, 1.348) | 0.774 | A, A², N, C |
| Diabetes adj. | 0.006 | A, A², B, N, C | 1093 | 0.915 | (0.646, 1.296) | 0.616 | A, A², B, |
| for BMI | | | | | | | N, C |
| Hypertension | 0.809 | A, A², N, C | 1167 | 1.072 | (0.834, 1.377) | 0.587 | A, A², N, C |

Boldface represents a P value < 2.17E−03.
*A = age, A² = age², S = sex, A × S = age × sex interaction,
A² × S = age² × sex interaction, B = log(BMI), N = nation, C = study (1990s vs 2000s)
^{†}Analysis conducted only in non-diabetics
^{‡}Leptin was not analyzed in men and women combined because
the distributions in each sex were very different.
Abbreviations:
s.e., standard error;
OR, odds ratio;
95% CI, 95% confidence interval;
circ., circumference;
adj., adjusted

Higher BMI and adiposity are usually associated with greater insulin resistance (higher fasting insulin and HOMA-IR), an atherogenic lipid profile (especially, higher serum triglycerides and lower HDL cholesterol), and lower adiponectin. It is, therefore, expected rs373863828's BMI-increasing allele (A) to also be associated with these metabolic variables. However, even though the A allele was consistently associated with higher BMI and adiposity in both discovery and replication cohorts, the expected associations with the above obesity-related comorbidities were not observed, and in some cases, were even reversed (Table 4, Table 5). Notably, when considering all subjects, the risk of diabetes was actually lower (OR 0.586 for discovery cohort, p=6.68E-09) or trended lower (0.742 for replication cohort, p=0.029) in carriers of the A allele. Likewise, even in non-diabetic subjects, the variant was associated with a small but significant reduction in fasting glucose in both cohorts (i.e., decrease of −2.25 mg/dL and −2.09 mg/dL for each copy of the A allele in the discovery and replication cohorts, respectively). These effects became even more significant after adjusting for BMI, suggesting an independent effect of the variant on glucose homeostasis and diabetes risk. Such effects could be due to survival bias, however no correlation between age and genotype was observed (linear regression P=0.849). These effects appear to be independent of obesity associated insulin resistance since associations with fasting insulin and HOMA-IR were not consistently observed across cohorts (higher only in replication cohort before adjusting for BMI). Furthermore, although the variant was associated with lower total cholesterol in the discovery cohort, consistent effects on serum lipids or adiponectin were likewise not observed. Together, these data suggest that the missense variant does not promote, and may even protect against, obesity-associated comorbidities, however additional studies will be required to confirm these findings and directly test this hypothesis.

Although the majority of genes contributing to obesity do so by influencing the central regulation of energy balance¹⁸, emerging evidence highlights the contribution of altered cellular metabolism to obesityl⁹. Therefore, the impact of rs373863828 on cellular bioenergetics was examined. To do so, an established 3T3-L1 adipocyte model was selected for two reasons: 1) CREBRF is widely expressed in virtually all tissues including adipose tissue (Supplementary FIG. 5), suggesting a fundamental cellular function, and 2) several CREB family proteins have been linked to mitochondrial function and metabolic phenotypes in adipocytes²⁰⁻²³. Thus, this model is well-suited to assess multiple potentially-relevant metabolic phenotypes.

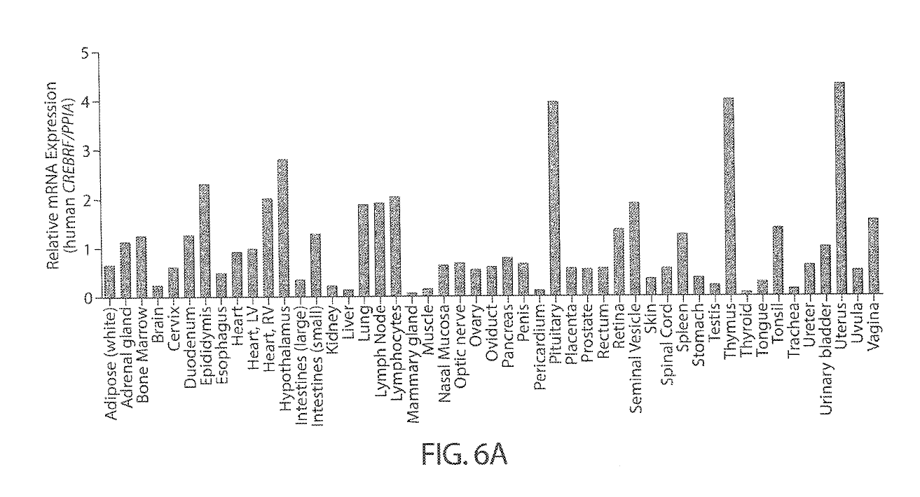

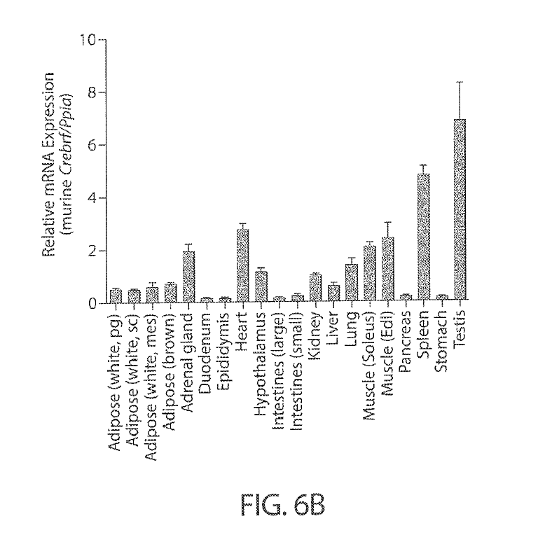

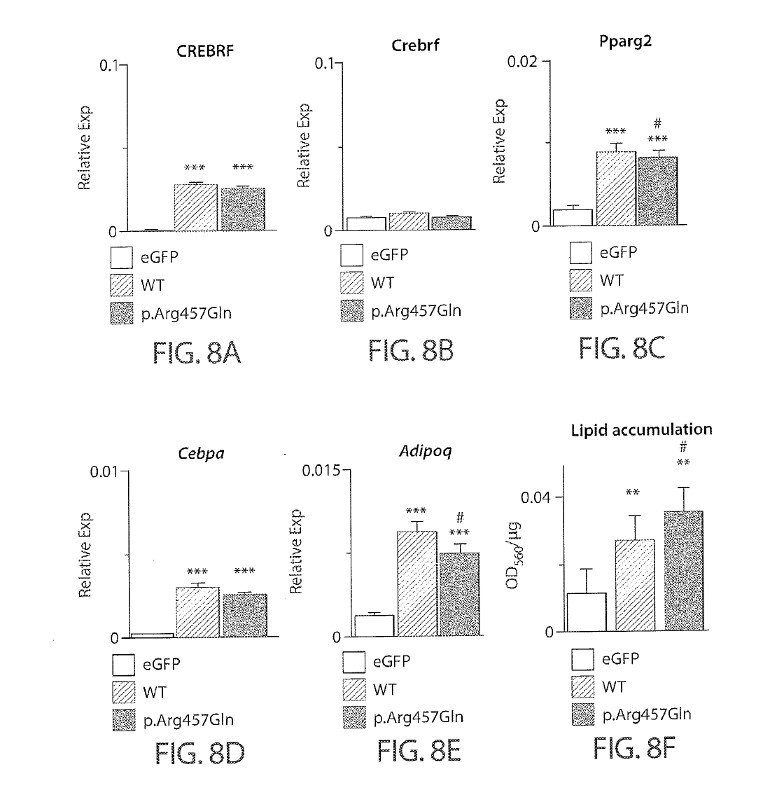

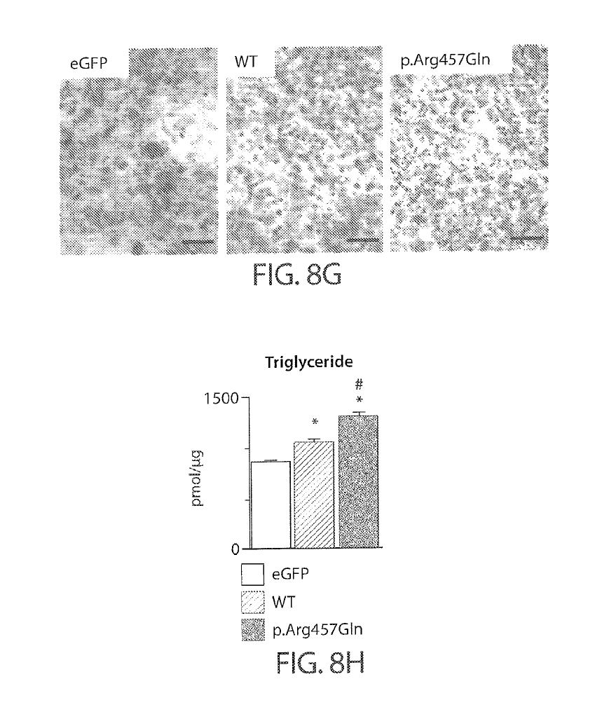

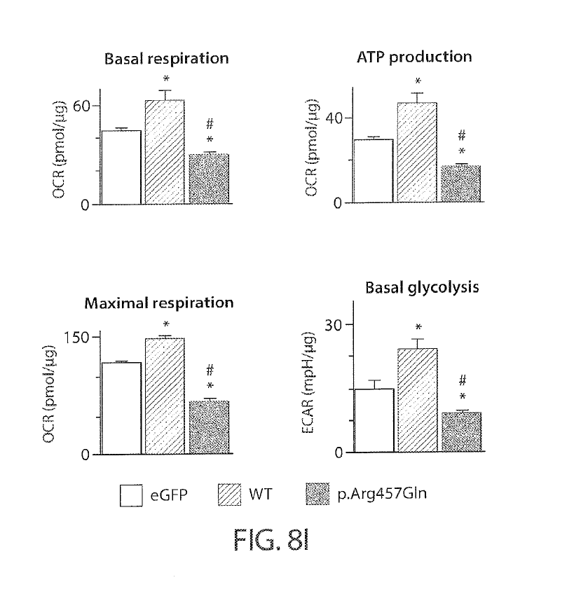

CREBRF is conserved and widely expressed (FIG. 6), consistent with an important cellular function. As several genes of the CREB family have been linked to adipogenesis²¹⁻²³, endogenous expression of Crebrf was first characterized, as well as effects of ectopic overexpression of human WT (NM_153607.2) or p.Arg457Gln CREBRF, in mouse 3T3-L1 preadipocytes, cells that differentiate into adipocytes following hormonal stimulation. Crebrf mRNA was indeed highly induced during adipogenesis in conjunction with adipogenic markers (Cebpa, Pparg, Adipoq), suggesting a role for CREBRF in this process (Figure. 7). Indeed, comparable stable overexpression of human WT or p.Arg457G CREBRF (FIG. 8A) (without changing endogenous Crebrf, FIG. 8B) was sufficient to induce adipogeneic markers (FIGS. 8C-E) and promote lipid/triglyceride accumulation (FIGS. 8F-H) in the absence of the standard hormonal induction of adipogenesis. However, even through p.Arg457Gln CREBRF resulted in slightly lower expression of adipogenic markers (FIGS. 8C and 8E), it promoted significantly (P=0.02) greater lipid/triglyceride accumulation compared to WT CREBRF (FIGS. 8F and 8G), indicating an independent effect of this variant on lipid accumulation.

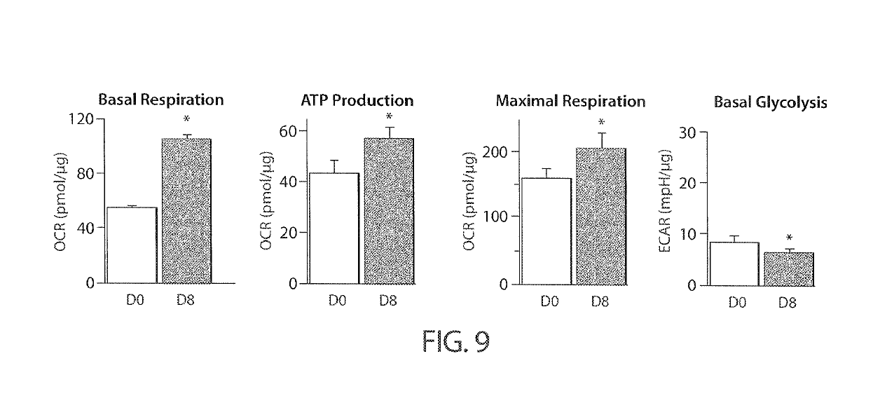

Since obesity is generally viewed as a disorder of energy homeostasis, and energy utilization (i.e. oxidative phosphorylation) increases during adipogenesis (FIG. 9)²⁴, the same 3T3-L1 cellular model was next used to assess whether the p.Arg457Gln CREBRF variant might enhance lipid accumulation by decreasing cellular energy metabolism. To determine if this increased energy storage was associated with decreased energy utilization, glycolysis, mitochondrial respiration and ATP production were next assessed. Glycolysis is suppressed and mitochondrial respiration and ATP production is enhanced by hormonally induced adipogenic differentiation^{24,25} (Supplementary FIG. 9). Overexpression of WT CREBRF increased whereas p.Arg457Gln CREBRF decreased multiple measures of cellular energy utilization including basal and maximal mitochondrial respiration, mitochondrial ATP production, and basal glycolysis (FIG. 8D). These data indicate that p.Arg457Gln CREBRF promotes more lipid accumulation while using less energy than WT CREBRF, supporting the notion that p.Arg457Gln is a “thrifty” variant that favors lipid storage over energy production.

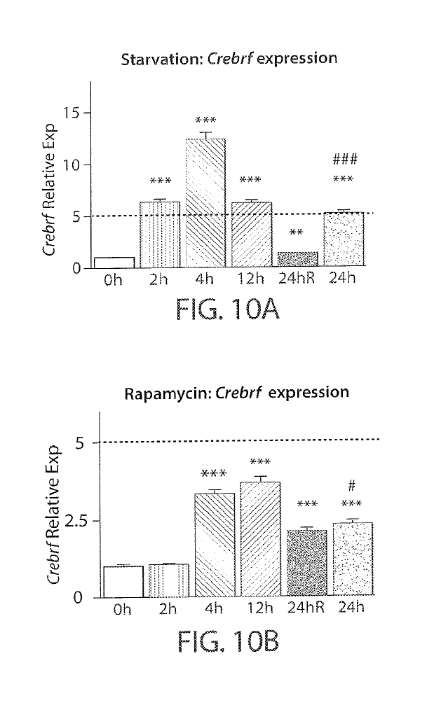

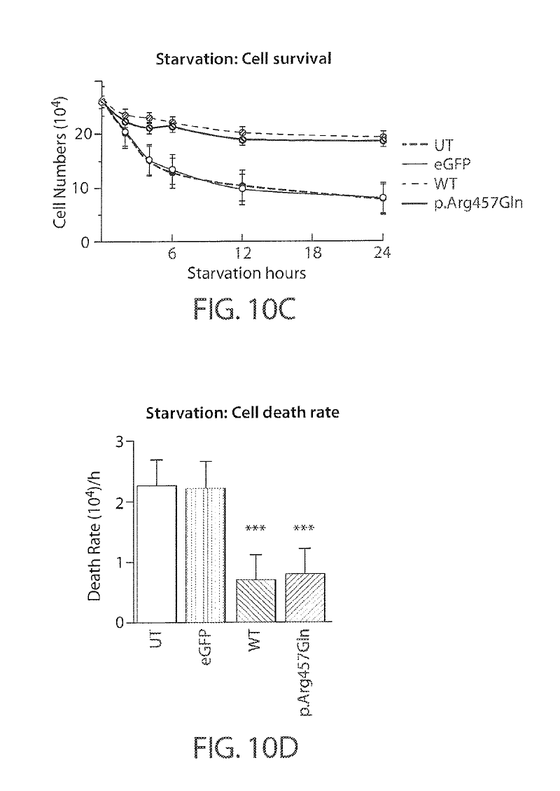

In addition to its role in in cellular energy storage and utilization, the Drosophila CREBRF ortholog, Reptor, has recently been implicated in both cellular and organismal adaptation to nutritional stress by mediating the downstream transcriptional response of the cellular energy sensor TORC1^{26,27}. In support of this hypothethesis, CREBRF orthologs are highly induced/activated upon starvation in all tissues of Drosophila^{26,27} as well as in human lymphoblasts^{28,29}. Moreover, both Reptor knockout flies²⁶ and Crebrf knockout mice³⁰ have lower total energy storage and body weight, respectively. Similarly, nutrient starvation of 3T3-L1 cells rapidly increased Crebrf mRNA expression, which peaked at 13-fold by 4 h (P=1.1×10⁻¹⁶), and remained 5-fold elevated at 24 h.(P=4.1×10⁻¹⁴) (FIG. 10A). Treatment with rapamycin, a TORC1 inhibitor, also rapidly increased Crebrf mRNA expression but less than starvation (FIG. 10B), indicating that additional TORC1-independent signals converge on Crebrf. In addition, overexpression of WT and p.Arg457Gln CREBRF equally reduced the rate of cell death to ˜1/3 of controls within the first 6 hours in nutrient starved 3T3-L1 preadipocytes (FIGS. 10C and 10D). These data indicate CREBRF is a starvation responsive gene and that overexpression of WT and p.Arg457Gln CREBRF confer similar protection against cellular nutritional stress.

The transcription factor binding sites in the CREBRF gene were analyzed and significant enrichment of binding sites for transcription factors were found. These transcription factors involve in a range of biological processes as shown in Table 6. This analysis was performed using the PANTHER Classification System at http://www.pantherdb.org. This tool classifies transcription factor binding sites within a query gene (in this case CREBRF) according to the gene ontology annotations for each transcription factor. Statistical analysis for enrichment of transcription factor binding sites for each gene ontology (GO) group is performed to compare the enrichment compared to the assumption of random distribution of binding sites within the genome. For example, 2-fold enriched means that there are twice as many binding sites for the transcription factors within that particular GO category in the CREBRF gene as would be under the assumption of random distribution of those binding sites. Table 6 shows the gnes upstream of CREBRF. The p value is the statistical significance of this fold enrichment.

TABLE 6
Genes upstream of CREBRF

| | Fold | | Factors (number of |
|---|---|---|---|
| GO category | enriched | p | binding sites) |
| Positive regulation of | 200 | 9.77E−05 | USF1 (5), USF2 (2), |
| transciption by glucose | | | SRF (2) |
| Stress response | 18 | 1.11E−03 | EP300 (8), CEBPB (7), |
| | | | EGR1 (5) |
| Skeletal muscle cell | 18 | 9.05E−03 | FOS (7), EGR1 (5), |
| differentiation | | | PAX5 (4) |
| Steroid hormone mediated | 18 | 1.11E−02 | HNF4G (3), NR2F2 (2), |
| signaling pathway | | | NR3C1 (2) |
| Response to cAMP | 16 | 7.71E−05 | EP300 (8), FOS (7), |
| | | | EGR1 (5) |
| Skeletal muscle tissue | 13 | 5.19E−05 | EP300 (8), FOS (7), |
| development | | | EGR1 (5) |
| Cellular response to | 12 | 5.69E−06 | FOS (7), MYC (6), |
| transforming growth factor | | | CREB1 (4) |
| beta stimulus | | | |
| Response to estrogen | 8 | 2.53E−02 | EP300 (8), GATA3 (6), |
| | | | FOXA1 (6) |
| Hemopoiesis | 8 | 8.36E−11 | RUNX3 (8), CHD2 (6), |
| | | | GATA2 (6) |
| Response to lipid | 5 | 1.30E−11 | EP300 (8), CEBPB (7), |
| | | | FOS (7) |
| Mitochodrial biogenesis | | | MYC (6), YY1 (5), |
| | | | GABPA (3), NRF1 (1) |

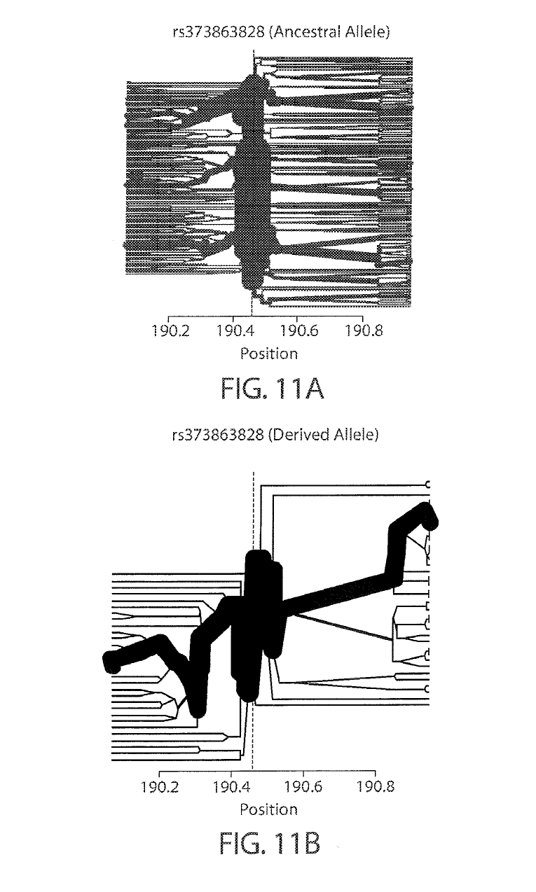

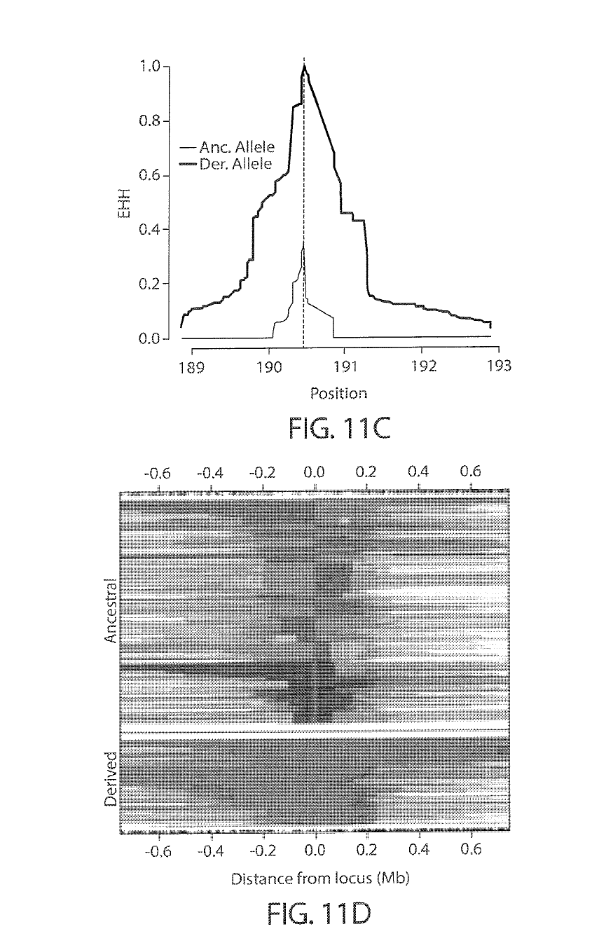

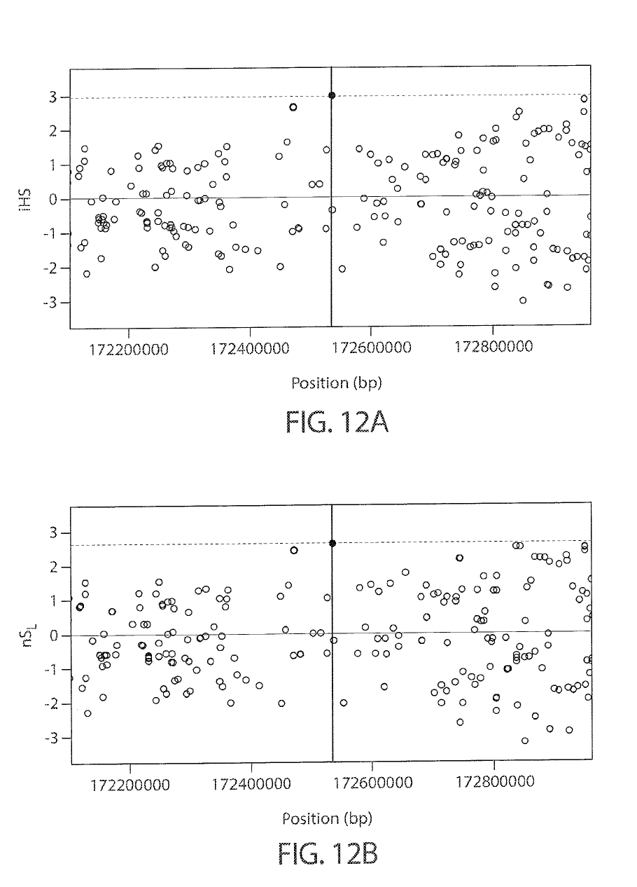

Complementing the functional evidence of “thriftiness”, evidence of positive selection at the missense variant in Samoan genomes was identified. The core haplotype carrying the derived BMI-increasing allele showed long-range linkage disequilibrium (as shown by the single thick branch in FIG. 11B vs. FIG. 11A), and had elevated extended haplotype homozygosity (EHH) relative to those haplotypes carrying the ancestral allele (FIG. 11C). Haplotypes carrying the derived allele extend longer than haplotypes carrying the ancestral allele (FIG. 11D). Evidence of positive selection is indicated by the integrated haplotype score (iHS) of 2.94 (P≈0.003) and the nSL score of 2.63 (P≈0.008) (FIG. 12).

In 1962 James Neel posited the existence of a thrifty gene that provides a metabolic advantage in times of famine and promotes metabolic diseases in times of nutritional excess³¹. By carrying out a genome-wide association of BMI in the Samoan population, a strongly associated missense variant in CREBRF with a much larger effect size than any other known common BMI risk variant was discovered and replicated. Functional evidence further demonstrates that this missense variant promotes cellular energy conservation by increasing fat storage and decreasing energy utilization in an adipocyte model compared to WT. The potential importance of this variant in organismal energy homeostasis is further supported by the “lean” phenotype of mice³⁰ and flies²⁶ lacking this gene. These data, in combination with evidence of positive selection, support a “thrifty” variant hypothesis for human obesity and underscore the value of examining unique populations to identify novel genetic contributions to complex traits.

This variant was not detected by previous large-scale genome-wide association scans because it is extremely rare in most other populations. In Samoans, the risk allele has a much larger effect on BMI than other common BMI-associated loci found to date. In a model system, the p.Arg457Gln risk variant increases lipid accumulation while limiting energy utilization, but providing the same protection from nutritional stress as WT CRBRF does. Together, these data support an important role for CREBRF in energy homeostasis, thereby identifying a novel pathway for therapeutic intervention in metabolic disease. Further studies of CREBRF are likely to reveal important new insights into the pathogenesis of obesity, nutrition partitioning, and the adaptive response to starvation. Future studies of obesity and other metabolic phenotypes should include its potential modifying and mediating influences with diet and physical activity and gene-gene interactions. The present studies cannot determine the evolutionary source of this variant or resolve questions about the roles of selection and drift in determining its frequency. Detailed anthropological genetic studies throughout the Pacific may help clarify this. Lastly, research is urgently needed about how to integrate and use knowledge of this obesity risk variant to benefit Samoans at both the individual and population health levels.

### Example 2. CREBRF Knockdown Produces Opposite Effects to WT Overexpression

To determine the effect of loss of function of CREBRF polypeptide, 3T3-L1 adipocytes were transfected with an inducible shRNA construct targeting the Crebrf mRNA. Table 7 lists the shRNA clones and the gene target sequence of each clone.

TABLE 7
shRNA clones and the gene targeting
sequence of each clone

| shRNA clone | Gene targeting sequence |
|---|---|
| V3SM7671-235834732 | TGGTTAACAAATTCTGAGG |
| V3SM7671-235231855 | AGGTATCTCGATTCCACTC |
| V3SM7671-233788864 | TGGAGTTTTACTGATGACC |

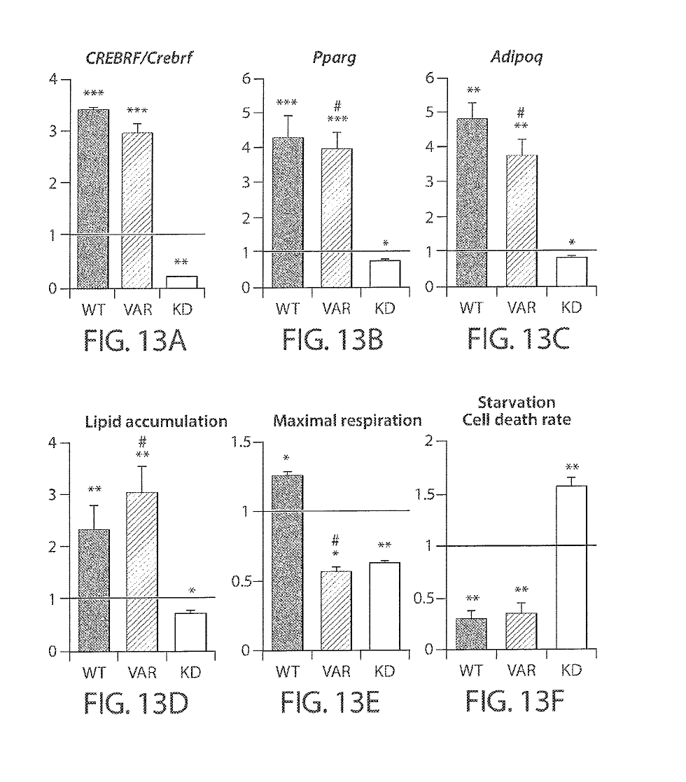

The oligonucleotide encoding the shRNA was cloned into the SMARTvector inducible lentiviral shRNA vector (GE Life Science). The vector contains a TRE3G tetracycline inducible promoter. The transcription of the shRNA was induced by doxycycline. The expression of shRNA (V3SM7671-235834732) suppresses the expression of wild type and variant CREBRF gene (FIG. 13A) as well as adipogenic marker Pparg (FIG. 13B) and Adipoq (FIG. 13C). The CREBRF knockdown results in reduced lipid accumulation (FIG. 13D) and maximal respiration (FIG. 13E) while renders cells susceptible to death induced by starvation (FIG. 13F). These data indicate that CREBRF knockdown produces opposite effects to wild type CREBRF overexpression.

To investigate the function of the CREBRF domain in which the p.Arg457Gln variant is located, recombinant 3T3-L1 cells were generated, in which the exon 5 of the CREBRF gene, where the p. Arg457Gln is located, was deleted from the genome (the endogenous CREBRF gene locus) of the cell. For CRISPR mutagenesis, the protocols published by Feng Zhang's group (Nat Protoc. 2013 November; 8(11): 2281-308) was modified. Briefly, vector PX459 was obtained from Addgene. Plasmid vector pSpCas9-2A-Puro(PX459) (Addgene) was linearized by digestion with Bbsl. Annealed oligos, served as inserts, were phosphorylated and annealed by using T4 PNK (NEB). Ligation reaction were performed by T4 DNA Ligase (NEB). The plasmids with the oligonucleotide inserts were transformed into bacteria, individual clones were picked, the plasmid DNA was isolated and the correct insertion of the oligonucleotides was confirmed by agarose gel electrophoresis and DNA sequencing. Recombinant plasmid vectors were transfected into 3T3-L1 cells using the Lipofectamine 2000 regent (Invitrogen). Cell cloning was performed by limiting dilution in 96 well plates and individual clones were expanded and the CREBRF gene was analyzed for mutations induced by CRISPR/Cas9. Two weeks after transfection the cells were subjected to cloning by limiting dilution. Out of 11 clones analyzed, one clone (531C g.20,764_21,067 del304) had a complete deletion of exon 5. The deletion of exon 5 is expected to inactive the CREBRF gene's “thriftiness” function. Table 8 lists the guide RNA sequence for CRISPR/Cas9 mutagenesis targeting exon 5.

TABLE 8
The sequences of the insert on
the CRISP/Cas9 vector targeting exon 5

| Guide RNA | Sequence |
|---|---|
| gRNA mCrebrf e5-1 sense | CACCGGGATTCTGAGGCCTTCTGAG |
| gRNA mCrebrf e5-1 | AAACCTCAGAAGGCCTCAGAATCCC |
| anti-sense | |
| gRNA mCrebrf e5-3 sense | CACCGGTATCTCGATTCCACTCAGA |
| gRNA mCrebrf e5-3 | AAACTCTGAGTGGAATCGAGATACC |
| anti-sense | |

Similar protocol was used to generate recombinant cells in which arginine is substituted by glutamine at amino acid position 457 in human CREBRF or its murine equivalent (amino acid position 458). Below is the sequence of the single strand oligonucleotide for knocking in the p.Arg457Gln variant:

GCACTAAATATTTTTCAAACCTCTTACCATGATGTAAGCCATTTTTCTG
GTACATATTACTTGGCAAGGTATCTTGATTCCACTCAGAAGGCCTCAGA
ATCCTCTCTTGCTGTGATGGTGTAAGCTGCTCACTATACTCCCAGA

The pSpCas9-2A-Puro(PX459) has the backbone of PX459 plasmid. The total vector size is about 9200 bp. The selectable marker is Puromycin. The size of insert hSpCas9-2A-Puro is about 6000 bp. The promoter for Cas9 is Cbh promoter and the promoter for the guide RNA is U6 promoter.

### Example 3. P. Arg457 Gln Variant Enhances the Binding of CREBRF to CREBL2 and CREBRF Binding of Target Gene Promoters

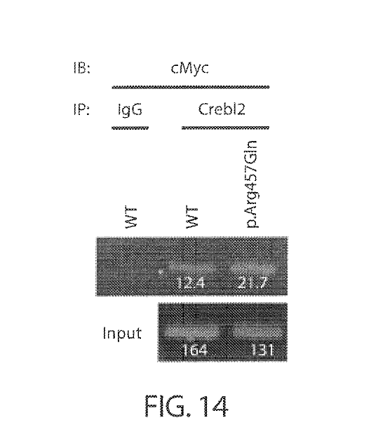

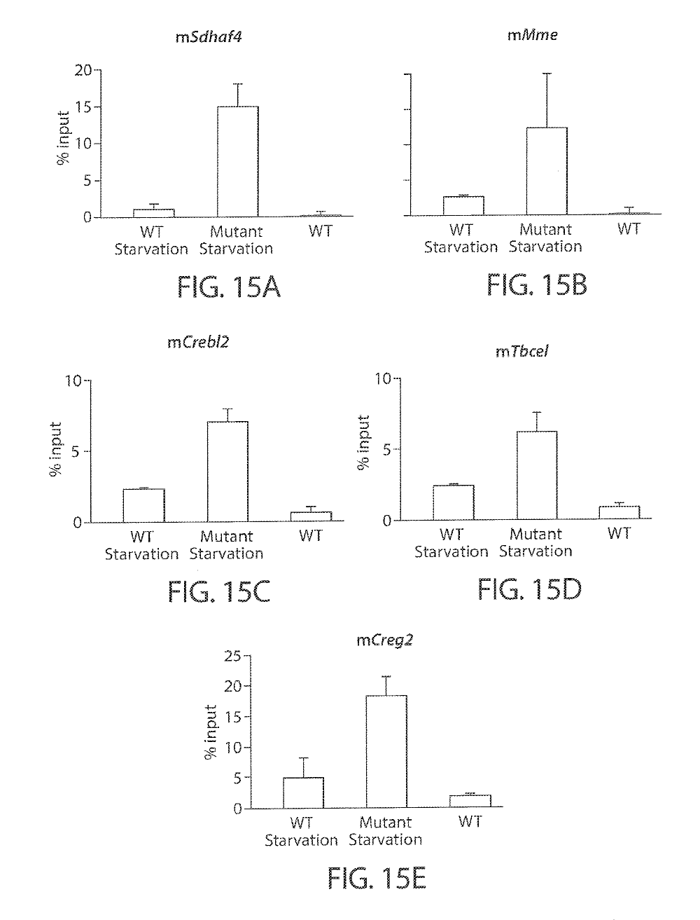

To investigate if the mutation of Arginine to Glutamine at the position 457 of the CREBRF protein has any effect, protein-protein and protein-DNA interactions of p. Arg457Gln and wild type CREBRF were assessed. Co-immunoprecipitation was conducted to show that CREBRF binds another transcription factor, CREBL2, and this binding is enhanced by the or p.Arg457Gln variant (FIG. 14).

By chromatin immunoprecipitation, several target genes that CREBRF can bind to were identified. Binding of CREBRF to these genes was enhanced by starvation, and further enhanced by the p.Arg457Gln variant (denoted as “mutation” in the x axis labels) (FIGS. 15A-15E).). Table 9 lists the oligonucleotides used for ChiP PCR.

TABLE 9
Oligonucleotides used for ChiP PCR

| Fruit Fly | Mouse | Binding | |
|---|---|---|---|
| Gene | ortholog¹ | Position | Primers |
| CG7224 | Sdhaf4 | promoter | F-CCGCTAATGCTTCTGTAGCC |
| | | | R-GATTACCCGAGGCAGTTGAG |
| CG9505 | Mme | promoter | F-TGGAAGCTGCTCTGCTATCG |
| | | | R-AAGTCCCATCCACATTGCTC |
| CG18619 | Crebl2 | promoter | F-GGAGATGGATGACAGCAAGG |
| | | | R-GGACATGAGGCACACTGGTA |
| CG12214 | Tbcel | promoter | F-GACAGGCACTTCTCCCAGAG |
| | | | R-TCAAGGGCATAGAGCAGTCC |
| CREG | Creg2 | promoter | F-CCCGTAAGAAGCGAAGTCTG |
| | | | R-CATTGAGCCTGAGCTGTGAA |

Sdhaf4 encodes succinate dehydrogenase complex assembly factor 4. Succinate dehydrogenase is a key mitochondrial enzyme complex linking the tricaboxylic cycle with the electron transport chain. Sdhaf4 facilitates the assembly of the enzyme complex. Positive regulation of Sdhaf4 by Crebrf is likely to increase the efficiency of mitochondrial respiration, and limit the production of reactive oxygen species associated with the activity of unassembled succinate dehydrogenase subunits.

Mine encodes membrane metalloendopeptidase. Also known as neprilysin, Mme is a zinc-dependent endopeptidase that inactivates several peptide hormones, including glucagon and bradykinin. Up-regulation of Mme by Crebrf is expected to result in reduced glucagon availability and changes in glucose homeostasis.

Crebl2 encodes cAMP responsive element binding protein like 2. As indicated by our co-immunoprecipitation studies and investigations of Crebrf and Crebl2 orthologs in Drosophila (Tiebe et al. 2015) Crebl2 is a transcription factor and binding partner of Crebrf. The presence of Crebrf binding sites in the Crebl2 promoter provides evidence for transcriptional positive feedback regulation of the Crebrf/Crebl2 complex.

Tbcel encodes tubulin folding cofactor E like. Tbcel is a homolog of tubulin folding cofactors that depolymerizes tubulin microtubules. Thus Tbcel can regulate cell shape, cell division, the trafficking of cellular organelles, the secretion of proteins.

Creg2—cellular repressor of E1A-stimulated genes 2. Creg2 is a secreted glycoprotein highly expressed in neurons with little available functional data (only 1 paper in pubmed). It is likely involved in cellular differentiation.

### Example 4. Knock-in Cell and Mouse are Generated

The endogenous CREBRF sequence has been manipulated at the genomic level (i.e. not via an expression vector) to introduce the a nucleotide change that results in the arginine to glutamine substitution at amino acid position 457 in human CREBRF or its murine equivalent (amino acid position 458). The Arg457Gln variant or its equivalent in other model species can be introduced at the genomic level in cells and animals using a variety of techniques such Crispr/Cas9, BAC recombineering or any other techniques known in the art.

The below methods using CRISPR/Cas9 system can be used to generate a knockin of the CREBRF variant in any murine cell type, and has been successfully used to knockin the variant in cell and mice (CREBRF knockin mice).

The sequence comparison between the wild type (WT) Crebrf and p. Arg457Gln (Mut) is shown below:

WT:
CAAgGTATCTcGATTCC
Mut:
CAAtGTATCTtGATTCC

For mCrebrf, the Sequence submitted for guide is:

ttacaccatcacagcaagagaggattctgaggccttctgagtggaatcg
agataccttgccaagtaatatgtaccagaaaaatggcttacatcatg

Two guide primers have the sequence as follows:

| | |
|---|---|
| 1 | ctggtacatattacttggcaagg |
| 6 | ggattctgaggccttctgagtgg |

One backup primer has the sequence as follows:

| | |
|---|---|
| 5 | tttttctggtacatattacttgg |

mCREBRF Guides are as follows:

The generic primer has sequence as follows:

GAAATTAATACGACTCACTATAGGNNNNNNNNNNNNNNNNNNNNGTT
TTAGAGCTAGAAATAGC

The mCREBRF_RG guide 1 has sequence as follows:

GAAATTAATACGACTCACTATAGGctggtacatattacttggcaGTTTT
AGAGCTAGAAATAG

### C

The mCREBRF_RG guide 6 has sequence as follows:

GAAATTAATACGACTCACTATAGGggattctgaggccttctgagtggGT
TTTAGAGCTAGAAATAGC

Primers are selected for using 400-700 bp of CREBRF for product in Primer3plus:

The forward primers are as follows:

>mCREBRF_F1
tttaatgcctggcaccattt
>mCREBRF_F2
tgacaattgtgggaccatgt

The reverse primers are as follows:

>mCREBRF_R1
gaacgaggcagaggattcaa
>mCREBRF_R2
agaaggagccgttgtgacag
>mCREBRF_R3
Ccacactgatggaagctgtg

Briefly, the above specific sgRNA were selected, in which the sequence does not have any potential off-targets with fewer than 3 mismatches in the whole genome. To introduce the mutation in the locus a 200 bp ssODN Ultramer (MT) note herein was used as the template for homology directed repair (HDR) of the double strand break (DSB) produce by the CRISPR/Cas9 complex. The ultramer corresponds to the genomic sequence evenly flanking the target site, but contains substitutions that: i) introduce mutation, ii) introduce a new restriction site to facilitate genotyping and iii) mismatches in the seed sequences of the sgRNA to prevent further editing of the mutant allele by Cas9/sgRNA complex. It should be noted that if the DSB is repaired by non-homologous end joining instead of HDR, a frameshift could cause a premature stop codon and a null allele. Therefore, in the process of making the desired mice or cells, we will also generate a complete knockout (KO).

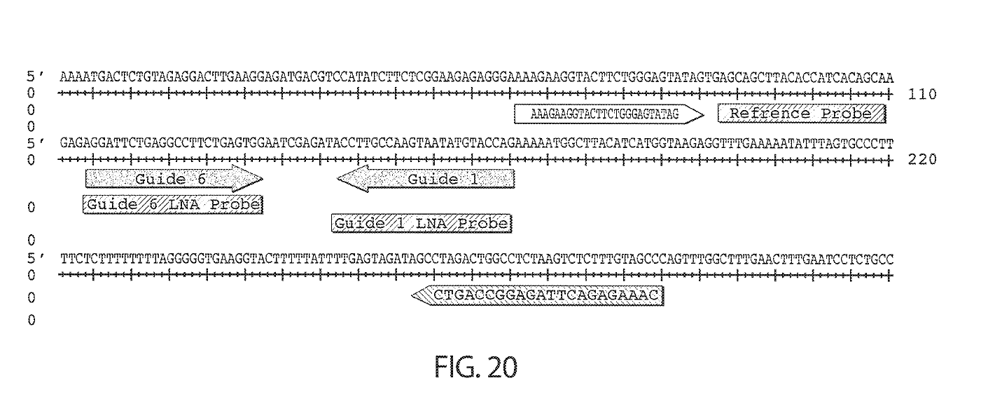

Cas9 mRNA and the sgRNA is produced according to Dr Gingras and co-worker optimized strategy (Pelletier S, Gingras S, Green DR. Mouse genome engineering via CRISPR-Cas9 for study of immune function. Immunity. 2015; 42(1):18-27. doi: 10.1016/j.immuni.2015.01.004. PubMed PMID: 25607456, Martinez J, Malireddi R K, Lu Q, Cunha L D, Pelletier S, Gingras S, Orchard R, Guan J L, Tan H, Peng J, Kanneganti T D, Virgin H W, Green DR. Molecular characterization of LC3-associated phagocytosis reveals distinct roles for Rubicon, NOX2 and autophagy proteins. Nat Cell Biol. 2015; 17(7):893-906. doi: 10.1038/ncb3192. PubMed PMID: 26098576). Briefly, Cas9 mRNA transcripts (capped and poly-adenylated) are produced from linearized plasmid encoding a human codon-optimized Cas9 nuclease using mMESSAGE mMACHINE T7 ULTRA Kit. The sgRNA is produced from the dsDNA template using the MEGAshortscript T7 Kit. Both Cas9 mRNA and sgRNAs are purified using the MEGAclear kit and eluted in nuclease-free water (all kits from Life Technologies). Table 10 below and FIG. 20 depict the strategy for detecting the above genetic manipulations in cells or cell/tissues from mice. The expected amplicon length is about 234 bp.

TABLE 10
Primers for detecting the knock in

| | Length | Tm | GC % |
|---|---|---|---|
| Forward | 25 | 61 | 40 |
| AAAGAAGGTACTTCTGGGAGTATAG (Sense) | | | |
| Reference Probe | 24 | 66 | 50 |
| AGCAGCTTACACCATCACAGCA (Sense) | | | |
| Reverse | 22 | 62 | 50 |
| CAAAGAGACTTAGAGGCCAGTC (AntiSense) | | | |

The guide 1 and guild 6 probe have the following characteristics as follows:

Guide 1 LNA Probe: ACCTT+G+C+C+AA+GT 67.0° C.

Guide 6 LNA Probe: CCTT+C+T+G+AGT+GG 66.0° C.

Mice were generated by the transgenic core at the University of Pittsburgh's department of immunology. Briefly, fertilized C57BL/6J embryos were microinjected with Cas9 mRNA (100 ng/μl), sgRNA (50 ng/μl) and ssODN (1 μM) and cultured overnight. The next day, 2-cell embryos were transferred to the oviducts of pseudo-pregnant CD1 female recipients. The above generally results in cutting efficiencies as high as 80% and HDR efficiency with ssODN at rate of 8 to 65%, demonstrating that the core can create mutant mice using the CRISPR/Cas9 technology. Tail genomic DNA is tested by PCR, restriction fragment length polymorphism (RFLP) and sequencing to identify putative founders. Similar approached as are used for embryos can be used to create variant-specific knockin in of virtually any cell type.

The practice of the CRISPR/Cas9 employs techniques that are explained fully in the literature, such as, Lin X, Pelletier S, Gingras S, Rigaud S, Maine C J, Marquardt K, Dai Y D, Sauer K, Rodriguez A R, Martin G, Kupriyanov S, Jiang L, Yu L, Green D R, Sherman L A. CRISPR-Cas9 mediated modification of the NOD mouse genome with Ptpn22R619W mutation increases autoimmune diabetes. Diabetes. 2016. doi: 10.2337/db16-0061. PubMed PMID: 27207523, Van de Velde L A, Gingras S, Pelletier S, Murray PJ. Issues with the Specificity of Immunological Reagents for Murine IDO1. Cell Metab. 2016; 23(3):389-90. doi: 10.1016/j.cmet.2016.02.004. PubMed PMID: 26959176, Wang H, Yang H, Shivalila C S, Dawlaty M M, Cheng A W, Zhang F, Jaenisch R. One-step generation of mice carrying mutations in multiple genes by CRISPR/Cas-mediated genome engineering. Cell. 2013; 153(4):910-8. doi: 10.1016/j.ce11.2013.04.025. PubMed PMID: 23643243; PMCID: PMC3969854, Bae S, Park J, Kim J S. Cas-OFFinder: a fast and versatile algorithm that searches for potential off-target sites of Cas9 RNA-guided endonucleases. Bioinformatics. 2014; 30(10):1473-5. doi: 10.1093/bioinformatics/btu048. PubMed PMID: 24463181; PMCID: PMC4016707. These techniques are applicable to the production of the knockin mice, and, as such, may be considered in making and practicing the invention.

### Example 5. CREBRF Regulates Homeostasis in Hepatocytes

Hepatocytes, like adipocyte, play a critical role in determining cellular and organismal energy homeostasis and energy substrate metabolism (i.e. obesity and its complications). As reported herein below, manipulation of CREBRF in hepatocytes results in qualitatively similar outcomes as observed in adipocytes and/or adipocyte precursors.

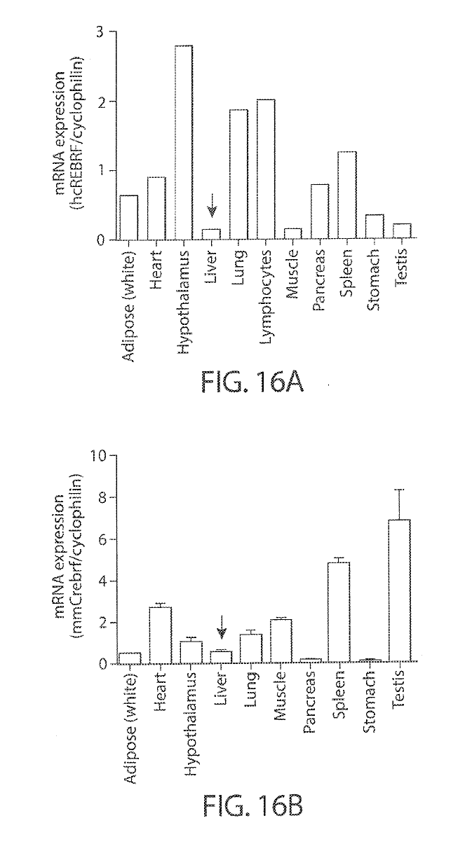

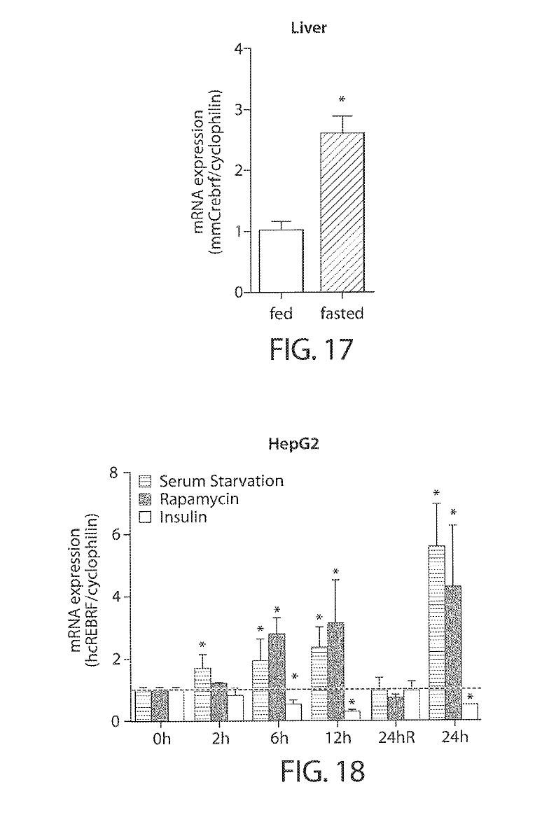

CREBRF is expressed in human liver (FIG. 16A) and murine liver (FIG. 16B). The expression of CREBRF is nutritionally-regulated in murine liver (FIG. 17). The mRNA level of CREBRF was increased in mice hepatocytes after fasting (FIG. 17). The results show that endogenous CREBRF is highly induced in response to fasting. CREBRF was induced by serum starvation and rapamycin (mTOR inhibition) and suppressed by insulin treatment in human HepG2 hepatocytes (FIG. 18). The results show that serum starvation and rapamycin induce CREBRF, whereas insulin and refeeding suppresses CREBRF in HepG2 hepatocytes.

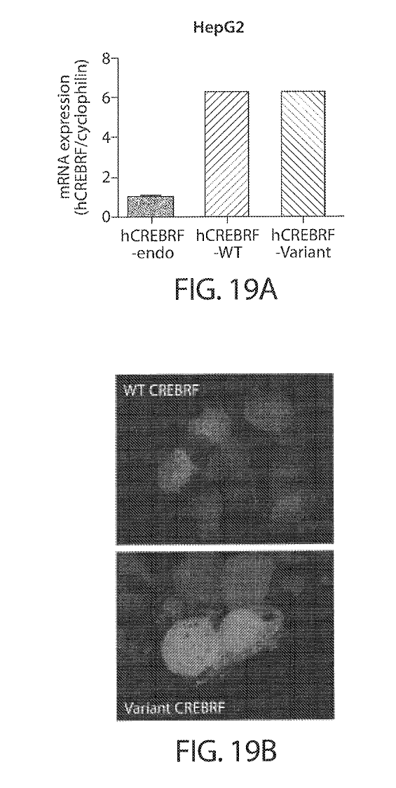

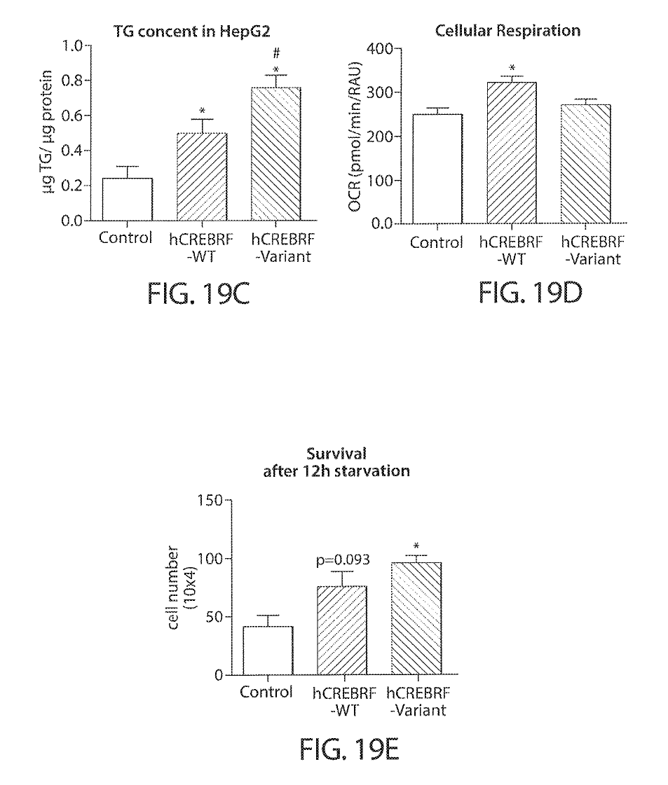

Overexpression of wild-type or variant (p. Arg457Gln) CREBRF influences hepatocellular lipid content, mitochondrial respiration, and cell survival (FIGS. 19A-19E). To determine the effects of overexpression of wild type and p.Arg457Gln on hapatocytes, HepG2 cells were transducted with 100 MOI adenovirus expressing hCREBRF-WT, hCREBRF-RQ, or GFP (Control). RQ stands for p. Arg457Gln variant. To determine the TG (triglyceride) content, two days after transduction, the cells were washed with 1×PBS and afterwards the lipids were extracted with hexane:isopropanol (3:2). After the solvent was evaporated, TG were determined using TG infinity Kit (ThermoScientific). To determine the protein concentration, cells were lysed with 0.3% SDS 0.1N NaOH for 6h. Afterwards the protein concentration was determined using BCA Kit (ThermoScientific) (n=6/construct). To determine the cellular respiration, cells were assayed using Seahorse technology to determine oxygen consumption rate. (1 uM oligomycin, 1 uM FCCP, and 5 uM Antimycin). For survival study, two days after transduction, the cells were treated with HBSS for 12h, afterwards the cells were collected and counted using a Hemacytometer (n=6/construct). These results indicate that the overexpression of wild type and p.Arg457Gln CREBRF in hepatocytes has similar effect as observed in adipocytes and/or adipocyte precursors.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

The following documents are cited herein.
1 Åberg, K. et al. Susceptibility loci for adiposity phenotypes on 8p, 9p, and 16q in American Samoa and Samoa. Obesity (Silver Spring) 17, 518-524 (2009).
2 Swinburn, B. A., Ley, S. J., Carmichael, H. E. & Plank, L. D. Body size and composition in Polynesians. Int J Obes Relat Metab Disord 23, 1178-1183 (1999).
3 Hawley, N. L. et al. Prevalence of adiposity and associated cardiometabolic risk factors in the Samoan genome-wide association study. Am J Hum Biol 26, 491-501 (2014).
4 Tishkoff, S. Strength in small numbers. Science 349, 1282-1283 (2015).
5 McGarvey, S. T., Bindon, J. R., Crews, D. E. & Schendel, D. E. in Human Population Biology: A Transdisciplinary Science (eds M. A. Little & J. D. Haas) 263-279 (Academic Press, 1989).
6 McGarvey, S. T. The thrifty gene concept and adiposity studies in biological anthropology. The Journal of the Polynesian Society 103, 29-42 (1994).
7 Zimmet, P., Dowse, G., Finch, C., Seijeantson, S. & King, H. The epidemiology and natural history of NIDDM—lessons from the South Pacific. Diabetes Metab Rev 6, 91-124 (1990).
8 Kirch, P. V. & Rallu, J.-L. in The Growth and Collapse of Pacific Island Societies (eds Patrick V. Kirch & Jean-Louis Rallu) Ch. 1, 1-14 (University of Hawaii Press, 2007).
9 Friedlaender, J. S. et al. The genetic structure of Pacific Islanders. PLoS Genet 4, e19 (2008).
10 Tsai, H.-J. et al. Distribution of genome-wide linkage disequilibrium based on microsatellite loci in the Samoan population. Human Genomics 1, 327-334 (2004).
11 Green, R. C. in The Growth and Collapse of Pacific Island Societies (eds Patrick V. Kirch & Jean-Louis Rallu) Ch. 11, 203-231 (University of Hawaii Press, 2007).
12 Exome Aggregation Consortium (ExAC). <http://exac.broadinstitute.org/variant/5-172535774-G-A>(2015).
13 Kichaev, G. et al. Integrating functional data to prioritize causal variants in statistical fine-mapping studies. PLoS Genet 10, e1004722 (2014).
14 Loos, R. J. & Yeo, G. S. The bigger picture of FTO: the first GWAS-identified obesity gene. Nat Rev Endocrinol 10, 51-61 (2014).
15 Speliotes, E. K. et al. Association analyses of 249,796 individuals reveal 18 new loci associated with body mass index. Nat Genet 42, 937-948 (2010).
16 Eicher, J. D. et al. GRASP v2.0: an update on the Genome-Wide Repository of Associations between SNPs and phenotypes. Nucleic Acids Res 43, D799-804 (2015).
17 Leslie, R., O'Donnell, C. J. & Johnson, A. D. GRASP: analysis of genotypephenotype results from 1390 genome-wide association studies and corresponding open access database. Bioinformatics 30, i185-194 (2014).
18 Locke, A. E. et al. Genetic studies of body mass index yield new insights for obesity biology. Nature 518, 197-206 (2015).
19 Pearce, L. R. et al. KSR2 mutations are associated with obesity, insulin resistance, and impaired cellular fuel oxidation. Cell 155, 765-777 (2013).
20 Vankoningsloo, S. et al. CREB activation induced by mitochondrial dysfunction triggers triglyceride accumulation in 3T3-L1 preadipocytes. J Cell Sci 119, 1266-1282 (2006).
21 Reusch, J. E., Colton, L. A. & Klemm, D. J. CREB activation induces adipogenesis in 3T3-L1 cells. Mol Cell Biol 20, 1008-1020 (2000).
22 Ma, X. et al. CREBL2, interacting with CREB, induces adipogenesis in 3T3-L1 adipocytes. Biochem J 439, 27-38 (2011).
23 Kim, T. H. et al. Identification of Creb314 as an essential negative regulator of adipogenesis. Cell Death Dis 5, e1527 (2014).
24 Wilson-Fritch, L. et al. Mitochondrial biogenesis and remodeling during adipogenesis and in response to the insulin sensitizer rosiglitazone. Mol Cell Biol 23, 1085-1094 (2003).
25 Keuper, M. et al. Spare mitochondrial respiratory capacity permits human adipocytes to maintain ATP homeostasis under hypoglycemic conditions. FASEB J 28, 761-770 (2014).
26 Tiebe, M. et al. REPTOR and REPTOR-BP Regulate Organismal Metabolism and Transcription Downstream of TORC1. Dev Cell 33, 272-284 (2015).
27 Stocker, H. Stress Relief Downstream of TOR. Dev Cell 33, 245-246 (2015).
28 Chen, R., Mallelwar, R., Thosar, A., Venkatasubrahmanyam, S. & Butte, A. J. GeneChaser: identifying all biological and clinical conditions in which genes of interest are differentially expressed. BMC Bioinformatics 9, 548 (2008).
29 Dengjel, J. et al. Autophagy promotes MHC class II presentation of peptides from intracellular source proteins. Proc Natl Acad Sci USA 102, 7922-7927 (2005).
30 Martyn, A. C. et al. Luman/CREB3 recruitment factor regulates glucocorticoid receptor activity and is essential for prolactin-mediated maternal instinct. Mol Cell Biol 32, 5140-5150 (2012).
31 Neel, J. V. Diabetes mellitus: a “thrifty” genotype rendered detrimental by “progress”? Am J Hum Genet 14, 353-362 (1962).
32 Pruim, R. J. et al. LocusZoom: regional visualization of genome-wide association scan results. Bioinformatics 26, 2336-2337 (2010).
33 Kampstra, P. Beanplot: A boxplot alternative for visual comparison of distributions. J Stat Softw 28, 1-9 (2008).
34 Gauderman, W. J. Sample size requirements for association studies of gene-gene interaction. Am J Epidemiol 155, 478-484 (2002).
35 Gauderman, W. J. Sample size requirements for matched case-control studies of gene-environment interaction. Stat Med 21, 35-50 (2002).
36 Scuteri, A. et al. Genome-wide association scan shows genetic variants in the FTO gene are associated with obesity-related traits. PLoS Genet 3, el 15 (2007).
37 McGarvey, S. T. Cardiovascular disease (CVD) risk factors in Samoa and American Samoa, 1990-95. Pacific Health Dialog 8, 157-162 (2001).
38 Deka, R. et al. Genetic characterization of American and Western Samoans. Hum Biol 66, 805-822 (1994).
39 McGarvey, S. T., Levinson, P. D., Bausser-Man, L., Galanis, D. J. & Hornick, C. A. Population change in adult obesity and blood lipids in American Samoa from 1976-1978 to 1990. Am J Hum Biol 5, 17-30 (1993).
40 Chin-Hong, P. V. & McGarvey, S. T. Lifestyle incongruity and adult blood pressure in Western Samoa. Psychosom Med 58, 131-137 (1996).
41 Galanis, D. J., McGarvey, S. T., Quested, C., Sio, B. & Afele-Fa´amuli, S. A. Dietary intake of modernizing Samoans: implications for risk of cardiovascular disease. J Am Diet Assoc 99, 184-190 (1999).
42 Dai, F. et al. Genome-wide scan for adiposity-related phenotypes in adults from American Samoa. Int J Obes (Lond) 31, 1832-1842 (2007).
43 Åberg, K. et al. A genome-wide linkage scan identifies multiple chromosomal regions influencing serum lipid levels in the population on the Samoan islands. J Lipid Res 49, 2169-2178 (2008).
44 Åberg, K. et al. Suggestive linkage detected for blood pressure related traits on 2q and 22q in the population on the Samoan islands. BMC Med Genet 10, 107 (2009).
45 Dai, F. et al. A whole genome linkage scan identifies multiple chromosomal regions influencing adiposity-related traits among Samoans. Ann Hum Genet 72, 780-792 (2008).
46 Keighley, E. D., McGarvey, S. T., Turituri, P. & Viali, S. Farming and adiposity in Samoan adults. Am J Hum Biol 18, 112-122 (2006).
47 Cole, T. J., Bellizzi, M. C., Flegal, K. M. & Dietz, W. H. Establishing a standard definition for child overweight and obesity worldwide: international survey. BMJ 320, 1240-1243 (2000).
48 American Diabetes Association. Diagnosis and classification of diabetes mellitus. Diabetes Care 35 Suppl 1, S64-71 (2012).
49 Matthews, D. R. et al. Homeostasis model assessment: insulin resistance and beta-cell function from fasting plasma glucose and insulin concentrations in man. Diabetologia 28, 412-419 (1985).
50 Laurie, C. C. et al. Quality control and quality assurance in genotypic data for genome-wide association studies. Genet Epidemiol 34, 591-602 (2010).
51 Purcell, S. et al. PLINK: a tool set for whole-genome association and populationbased linkage analyses. Am J Hum Genet 81, 559-575 (2007).
52 Aulchenko, Y. S., Ripke, S., Isaacs, A. & van Duijn, C. M. GenABEL: an R library for genome-wide association analysis. Bioinformatics 23, 1294-1296 (2007).
53 Heath, S. C. et al. Investigation of the fine structure of European populations with applications to disease association studies. Eur J Hum Genet 16, 1413-1429 (2008).
54 Conomos, M. P., Miller, M. B. & Thornton, T. A. Robust inference of population structure for ancestry prediction and correction of stratification in the presence of relatedness. Genet Epidemiol 39, 276-293 (2015).
55 International HapMap Consortium et al. Integrating common and rare genetic variation in diverse human populations. Nature 467, 52-58 (2010).
56 Manichaikul, A. et al. Robust relationship inference in genome-wide association studies. Bioinformatics 26, 2867-2873 (2010).
57 Albrechtsen, A., Nielsen, F. C. & Nielsen, R. Ascertainment biases in SNP chips affect measures of population divergence. Mol Biol Evol 27, 2534-2547 (2010).
58 Wollstein, A. et al. Demographic history of Oceania inferred from genome-wide data. Curr Biol 20, 1983-1992 (2010).
59 Hoffman, G. E. Correcting for population structure and kinship using the linear mixed model: theory and extensions. PLoS One 8, e75707 (2013).
60 Chen, W. M. & Abecasis, G. R. Family-based association tests for genomewide association scans. Am J Hum Genet 81, 913-926 (2007).
61 Devlin, B. & Roeder, K. Genomic control for association studies. Biometrics 55, 997-1004 (1999).
62 Therneau, T., Atkinson, E., Sinnwell, J., Schaid, D. & McDonnell, S. kinship2: Pedigree functions (2014).
63 R Core Team. R: A language and environment for statistical computing. (R Foundation for Statistical Computing, Vienna, Austria, 2014).
64 Winkler, T. W. et al. Quality control and conduct of genome-wide association metaanalyses. Nat Protoc 9, 1192-1212 (2014).
65 Willer, C. J., Li, Y. & Abecasis, G. R. METAL: fast and efficient meta-analysis of genomewide association scans. Bioinformatics 26, 2190-2191 (2010).
66 Cochran, W. G. The comparison of percentages in matched samples. Biometrika 37, 256-266 (1950).
67 Higgins, J. P. & Thompson, S. G. Quantifying heterogeneity in a meta-analysis. Stat Med 21, 1539-1558 (2002).
68 Higgins, J. P., Thompson, S. G., Deeks, J. J. & Altman, D. G. Measuring inconsistency in meta-analyses. BMJ 327, 557-560 (2003).
69 Delaneau, O., Marchini, J. & Zagury, J. F. A linear complexity phasing method for thousands of genomes. Nat Methods 9, 179-181 (2012).
70 Delaneau, O., Howie, B., Cox, A. J., Zagury, J. F. & Marchini, J. Haplotype estimation using sequencing reads. Am J Hum Genet 93, 687-696 (2013).
71 Delaneau, O., Zagury, J. F. & Marchini, J. Improved whole-chromosome phasing for disease and population genetic studies. Nat Methods 10, 5-6 (2013).
72 O'Connell, J. et al. A general approach for haplotype phasing across the full spectrum of relatedness. PLoS Genet 10, e1004234 (2014).
73 Delaneau, O., Marchini, J. & The 1000 Genomes Project Consortium. Integrating sequence and array data to create an improved 1000 Genomes Project haplotype reference panel. Nat Commun 5, 3934 (2014).
74 Marchini, J., Howie, B., Myers, S., McVean, G. & Donnelly, P. A new multipoint method for genome-wide association studies by imputation of genotypes. Nat Genet 39, 906-913 (2007).
75 Howie, B. N., Donnelly, P. & Marchini, J. A flexible and accurate genotype imputation method for the next generation of genome-wide association studies. PLoS Genet 5, e 1000529 (2009).
76 Marchini, J. & Howie, B. Genotype imputation for genome-wide association studies. Nat Rev Genet 11, 499-511 (2010).
77 Wang, X. et al. Evaluation of transethnic fine mapping with population-specific and cosmopolitan imputation reference panels in diverse Asian populations. Eur J Hum Genet (2015).
78 Gusev, A. et al. Low-pass genome-wide sequencing and variant inference using identity-by-descent in an isolated human population. Genetics 190, 679-689 (2012).
79 Aulchenko, Y. S., Struchalin, M. V. & van Duijn, C. M. ProbABEL package for genome-wide association analysis of imputed data. BMC Bioinformatics 11, 134 (2010).
80 Clayton, D. snpStats: SnpMatrix and XSnpMatrix classes and methods. R package v. 1.20.0 (2015).
81 Encode Project Consortium. An integrated encyclopedia of DNA elements in the human genome. Nature 489, 57-74 (2012).
82 Zebisch, K., Voigt, V., Wabitsch, M. & Brandsch, M. Protocol for effective differentiation of 3T3-L1 cells to adipocytes. Anal Biochem 425, 88-90 (2012).
83 Ramirez-Zacarias, J. L., Castro-Munozledo, F. & Kuri-Harcuch, W. Quantitation of adipose conversion and triglycerides by staining intracytoplasmic lipids with Oil red 0. Histochemistry 97, 493-497 (1992).
84 Bradford, M. M. A rapid and sensitive method for the quantitation of microgram quantities of protein utilizing the principle of protein-dye binding. Anal Biochem 72, 248-254 (1976).
85 Livak, K. J. & Schmittgen, T. D. Analysis of relative gene expression data using real-time quantitative PCR and the 2-AACT Method. Methods 25, 402-408 (2001).
86 Staples, J., Nickerson, D. A. & Below, J. E. Utilizing graph theory to select the largest set of unrelated individuals for genetic analysis. Genet Epidemiol 37, 136-141 (2013).
87 Staples, J. et al. PRIMUS: rapid reconstruction of pedigrees from genome-wide estimates of identity by descent. Am J Hum Genet 95, 553-564 (2014).
88 Cadzow, M. et al. A bioinformatics workflow for detecting signatures of selection in genomic data. Front Genet 5, 293 (2014).
89 Gautier, M. & Vitalis, R. rehh: an R package to detect footprints of selection in genome-wide SNP data from haplotype structure. Bioinformatics 28, 1176-1177 (2012).
90 Sabeti, P. C. et al. Detecting recent positive selection in the human genome from haplotype structure. Nature 419, 832-837 (2002).
91 Szpiech, Z. A. & Hernandez, R. D. selscan: an efficient multithreaded program to perform EHH-based scans for positive selection. Mol Biol Evol 31, 2824-2827 (2014).
92 Voight, B. F., Kudaravalli, S., Wen, X. & Pritchard, J. K. A map of recent positive selection in the human genome. PLoS Biol 4, e72 (2006).
93 Ferrer-Admetlla, A., Liang, M., Korneliussen, T. & Nielsen, R. On detecting incomplete soft or hard selective sweeps using haplotype structure. Mol Biol Evol 31, 1275-1291 (2014).

1. A recombinant cell comprising a promoter operably linked to a nucleic acid sequence encoding a CREB3 Regulatory Factor (CREBRF) polypeptide.

2. The recombinant cell of claim 2, wherein the cell is selected from the group consisting of a preadipocyte, an adipocyte, an hepatocyte and precursors thereof.

3. The recombinant cell of claim 2, wherein the cell is an adipocyte and is differentiated from a 3T3-L1 cell.

4. The recombinant cell of claim 3, wherein the promoter is an adipocyte specific promoter.

5. The recombinant cell of claim 2, wherein the cell is an hepatocyte.

6. The recombinant cell of claim 5, wherein the cell is an HepG2 cell.

7. The recombinant cell of claim 5, wherein the promoter is an hepatocyte specific promoter.

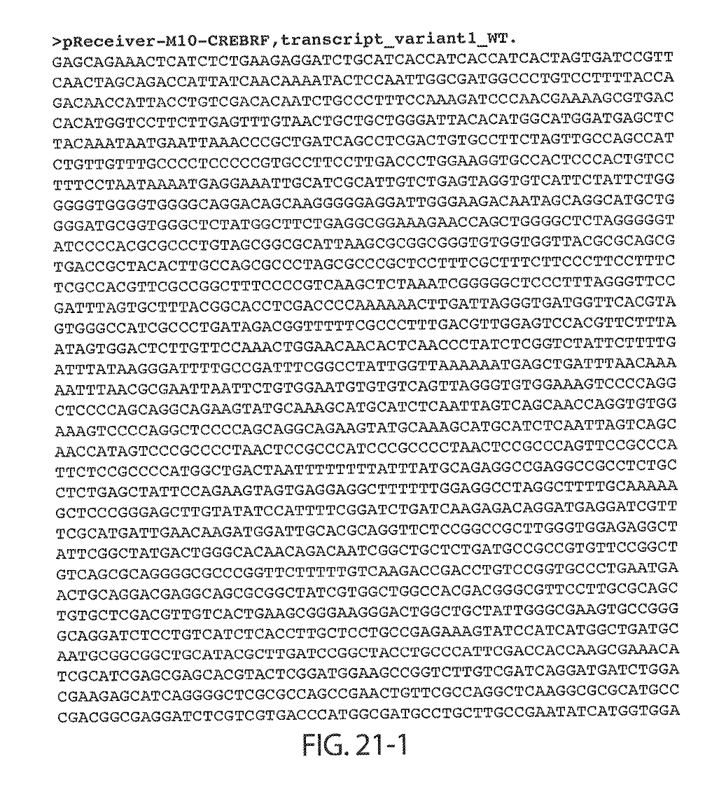

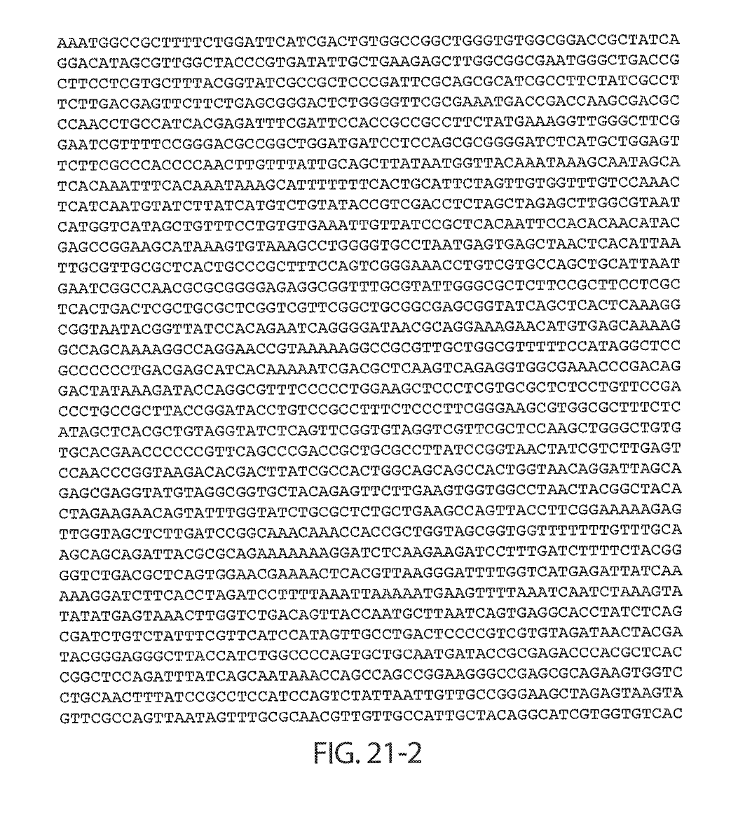

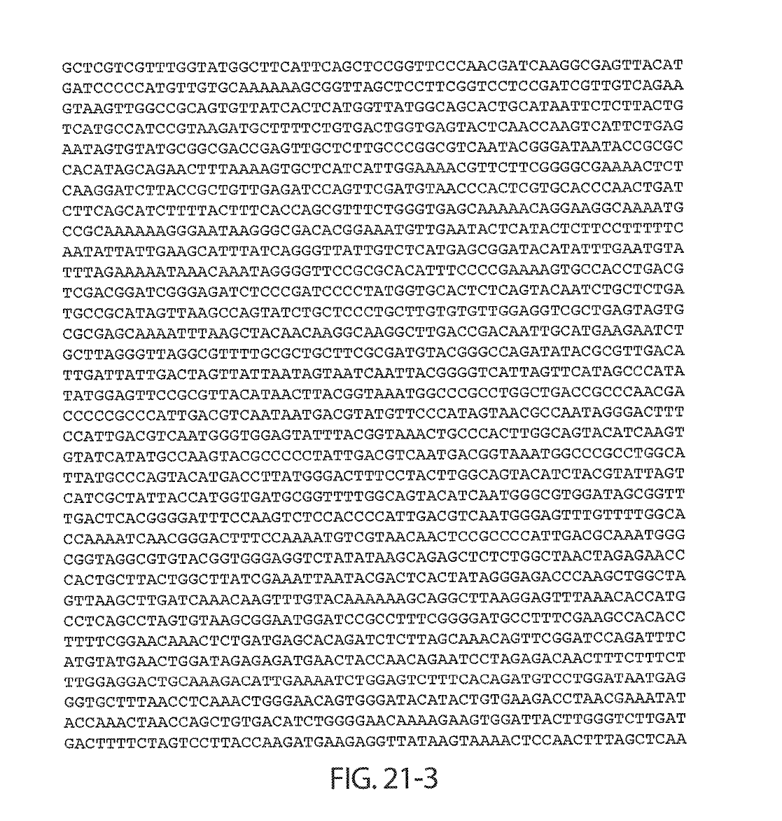

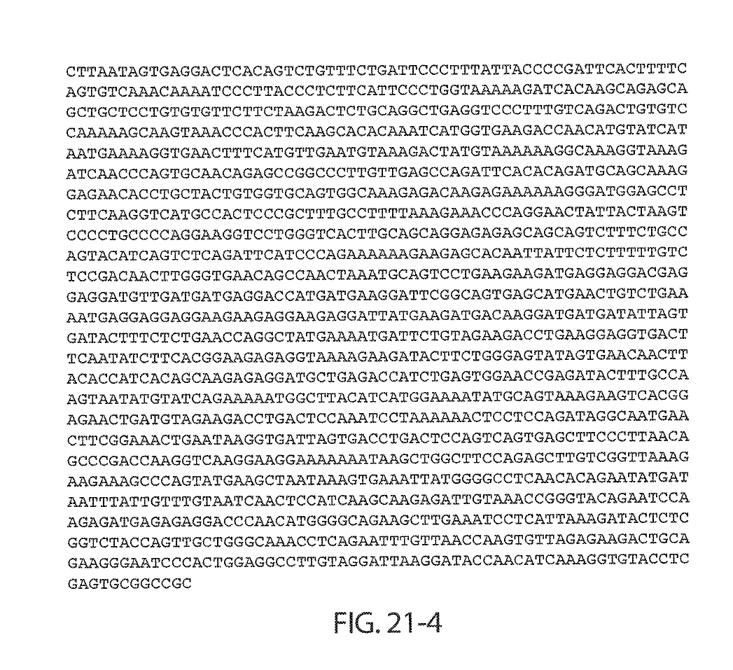

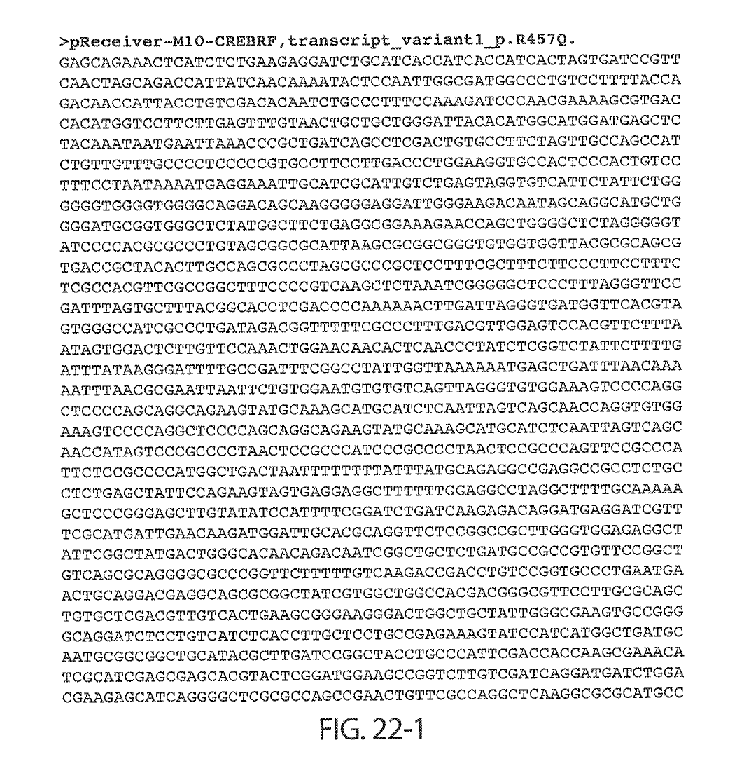

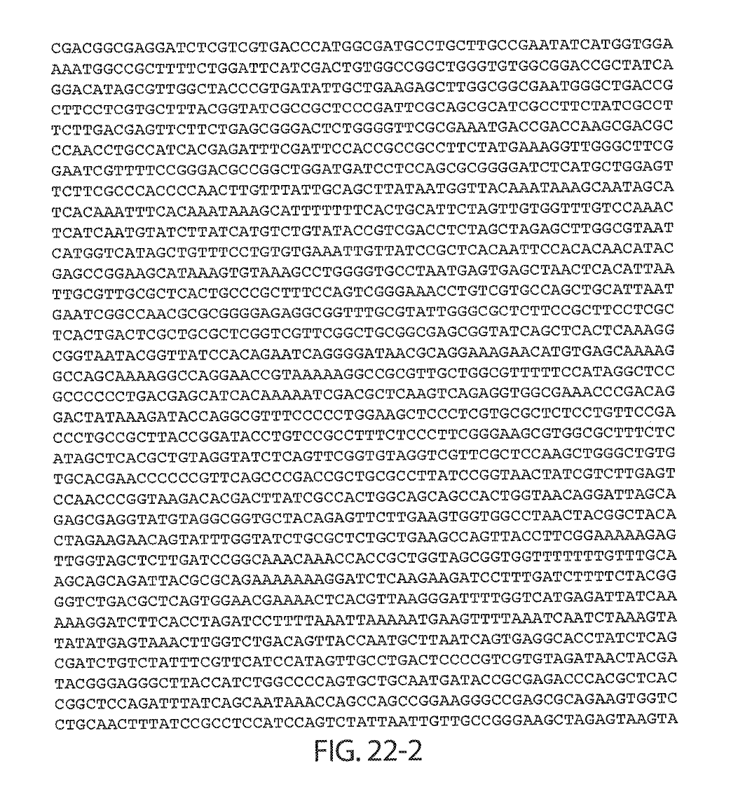

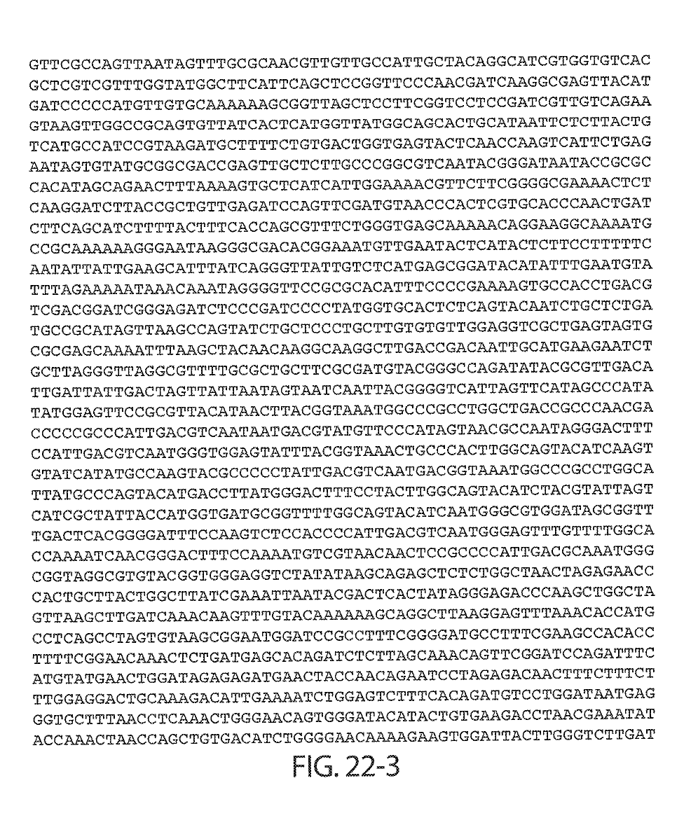

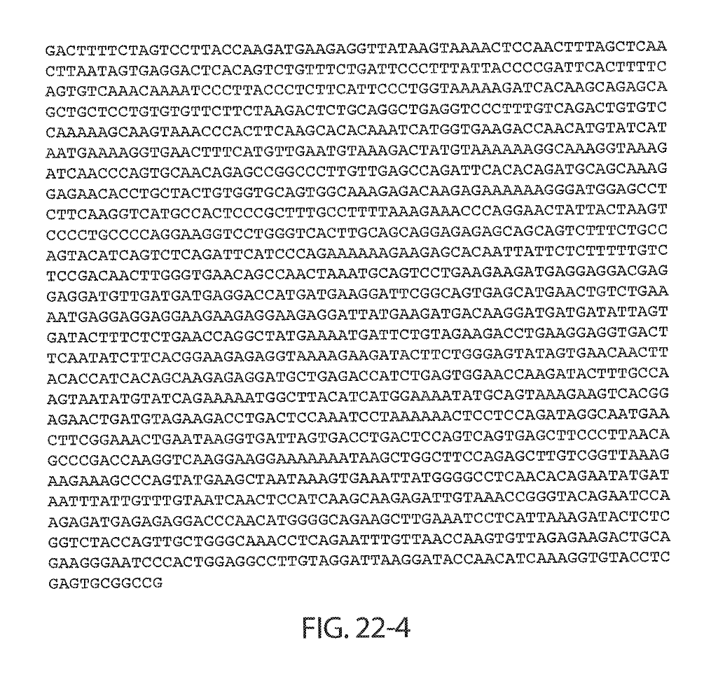

8. The recombinant cell of claim 1, wherein the nucleic acid comprises a nucleic acid sequence set forth in FIG. 21 or FIG. 22.

9. The recombinant cell of claim 1, wherein the nucleic acid encodes a mutant murine CREBRF polypeptide or a human CREBRF peptide.

10. The recombinant cell of claim 9, wherein the mutant CREBRF polypeptide comprises a substitution Arg457Gln.

11. The recombinant cell of claim 10, wherein the nucleic acid comprises a deletion of exon 5 of CREBRF gene.

12. The recombinant cell of claim 9, wherein the mutation is in the cell endogenous CREBRF locus.

13. The recombinant cell of claim 1, wherein the nucleic acid reduces or eliminates the expression of CREBRF polypeptide.

14. The recombinant cell of claim 1, wherein the nucleic acid encodes an inhibitory RNA against the CREBRF mRNA.

15. The recombinant cell of claim 1, wherein the nucleic acid encodes a shRNA against the CREBRF mRNA.

16. The recombinant cell of claim 1, comprising a CRISPR/Cas9 vector having the nucleic acid sequence, wherein the nucleic acid sequence targets the CREBRF gene.

17. The recombinant cell of claim 16, wherein the nucleic acid sequence guides the deletion of the exon 5 of the CREBRF gene.

18. The recombinant cell of claim 16, wherein the nucleic acid sequence guides the substitution of arginine at position 457 or its equivalent by glutamine.

19. An expression vector comprising a promoter operably linked to a nucleic acid sequence encoding a CREBRF polypeptide.

20-23. (canceled)

24. An expression vector comprising a CRISP/Cas9 module operably linked to a nucleic acid targeting against CREBRF gene.

25. (canceled)

26. (canceled)

27. A recombinant cell comprising the expression vector of claim 19.

28. A nucleic acid probe that specifically binds a nucleic acid encoding a CREB3 Regulatory Factor (CREBRF) polypeptide comprising a glutamine at amino acid position 457.

29-31. (canceled)

32. A knock-in mouse comprising a nucleic acid encoding a mutant murine CREB3 Regulatory Factor (CREBRF) polypeptide or a human CREBRF polypeptide.

33-36. (canceled)

37. A method of enhancing adipogenesis in a cell, increasing lipid accumulation in a cell or of making a cell resistant to starvation, the method comprising causing the cell to express or overexpress a CREB3 Regulatory Factor (CREBRF) polypeptide.

38-43. (canceled)

44. A method of genotyping a subject comprising contacting a cell of the subject with a nucleic acid probe of claim 28.

45. A method of identifying a subject as obese or at risk of obesity, the method comprising detecting one or more alleles encoding a CREB3 Regulatory Factor (CREBRF) polypeptide comprising a glutamine at amino acid position 457 in a biological sample from the subject, wherein the presence of one or more alleles encoding a CREBRF polypeptide comprising a glutamine at amino acid position 457 indicates that the subject is obese or is at risk of obesity.

46. A method of treating a subject identified as being obese or at risk of obesity, the method comprising administering the said identified subject a therapeutically effective amount of a compound that modulates adipogenesis in a cell of said subject, wherein the subject is identified as being obese or being at risk of obesity, the method comprising detecting one or more alleles encoding a CREB3 Regulatory Factor (CREBRF) polypeptide comprising a glutamine at amino acid position 457 in a biological sample from the subject, wherein the presence of one or more alleles encoding a CREBRF polypeptide comprising a glutamine at amino acid position 457 indicates that the subject is obese or is at risk of obesity.

47. (canceled)

48. (canceled)

49. A method of reducing adipogenesis or lipid accumulation in a cell, the method comprising reducing, eliminating or inactivating the adipogenic function of a CREBRF polypeptide in the cell.

50. A method of making a cell susceptible to starvation, the method comprising reducing, eliminating or inactivating the adipogenic function of a CREB3 Regulatory Factor (CREBRF) polypeptide.

51-56. (canceled)

57. A method of identifying a compound that modulates the expression of a CREB3 Regulatory Factor (CREBRF) polypeptide, comprising:
a) contacting a nucleic acid that expresses a CREBRF polypeptide with a compound under conditions suitable for expression by the nucleic acid;
b) determining the level of expression of the CREBRF polypeptide;
c) determining the level of expression of the nucleic acid in the absence of the compound; and
d) comparing the level of expression of the nucleic acid after contact with the compound with the level of expression of the nucleic acid without contact of the compound; thereby identifying a compound that modulates expression of the CREBRF polypeptide.

58-60. (canceled)

61. A method of identifying a compound that modulates adipogenesis, the method comprising contacting a recombinant cell of claim 1 and or the knock-in mouse of claim 32 with a compound, and assaying reporter expression in the contacted cell relative to a corresponding control cell, thereby identifying a compound that modulates adipogenesis.

62. A method of identifying a compound that modulate adipogenesis, the method comprising contacting a recombinant cell of claim 1 or the knock-in mouse of claim 32 with an shRNA against a gene of interest, and analyzing adipogenesis of the cell relative to a reference, thereby identifying an adipogenesis modulator.

63-68. (canceled)

69. A kit comprising an expression vector of claim 19 and instructions for use.

70. A kit comprising the nucleic acid probe of claim 28 and instructions for use.

71. A kit comprising the knock-in mouse of claim 32 and instructions for use.
